SEQUENCE LISTING (88 sequences)

SEQ ID NO: 1 - DNA, 1476 bp, Escherichia coli
atggcggtaacgcaaacagcccaggcctgtgacctggtcattttcggcgcgaaaggcgaccttgcgcgtcgtaaattgctgccttccctgtatcaactggaaaaagccggtcagctcaacccggacacccggattatcggcgtagggcgtgctgactgggataaagcggcatataccaaagttgtccgcgaggcgctcgaaactttcatgaaagaaaccattgatgaaggtttatgggacaccctgagtgcacgtctggatttttgtaatctcgatgtcaatgacactgctgcattcagccgtctcggcgcgatgctggatcaaaaaaatcgtatcaccattaactactttgccatgccgcccagcacttttggcgcaatttgcaaagggcttggcgaggcaaaactgaatgctaaaccggcacgcgtagtcatggagaaaccgctggggacgtcgctggcgacctcgcaggaaatcaatgatcaggttggcgaatacttcgaggagtgccaggtttaccgtatcgaccactatcttggtaaagaaacggtgctgaacctgttggcgctgcgttttgctaactccctgtttgtgaataactgggacaatcgcaccattgatcatgttgagattaccgtggcagaagaagtggggatcgaagggcgctggggctattttgataaagccggtcagatgcgcgacatgatccagaaccacctgctgcaaattctttgcatgattgcgatgtctccgccgtctgacctgagcgcagacagcatccgcgatgaaaaagtgaaagtactgaagtctctgcgccgcatcgaccgctccaacgtacgcgaaaaaaccgtacgcgggcaatatactgcgggcttcgcccagggcaaaaaagtgccgggatatctggaagaagagggcgcgaacaagagcagcaatacagaaactttcgtggcgatccgcgtcgacattgataactggcgctgggccggtgtgccattctacctgcgtactggtaaacgtctgccgaccaaatgttctgaagtcgtggtctatttcaaaacacctgaactgaatctgtttaaagaatcgtggcaggatctgccgcagaataaactgactatccgtctgcaacctgatgaaggcgtggatatccaggtactgaataaagttcctggccttgaccacaaacataacctgcaaatcaccaagctggatctgagctattcagaaacctttaatcagacgcatctggcggatgcctatgaacgtttgctgctggaaaccatgcgtggtattcaggcactgtttgtacgtcgcgacgaagtggaagaagcctggaaatgggtagactccattactgaggcgtgggcgatggacaatgatgcgccgaaaccgtatcaggccggaacctggggacccgttgcctcggtggcgatgattacccgtgatggtcgttcctggaatgagtttgagtaa

SEQ ID NO: 2 - DNA, 996 bp, Escherichia coli
atgaagcaaacagtttatatcgccagccctgagagccagcaaattcacgtctggaatctgaatcatgaaggcgcactgacgctgacacaggttgtcgatgtgccggggcaggtgcagccgatggtggtcagcccggacaaacgttatctctatgttggtgttcgccctgagtttcgcgtcctggcgtatcgtatcgccccggacgatggcgcactgacctttgccgcagagtctgcgctgccgggtagtccgacgcatatttccaccgatcaccaggggcagtttgtctttgtaggttcttacaatgcgggtaacgtgagcgtaacgcgtctggaagatggcctgccagtgggcgtcgtcgatgtggtcgaggggctggacggttgccattccgccaatatctcaccggacaaccgtacgctgtgggttccggcattaaagcaggatcgcatttgcctgtttacggtcagcgatgatggtcatctcgtggcgcaggaccctgcggaagtgaccaccgttgaaggggccggcccgcgtcatatggtattccatccaaacgaacaatatgcgtattgcgtcaatgagttaaacagctcagtggatgtctgggaactgaaagatccgcacggtaatatcgaatgtgtccagacgctggatatgatgccggaaaacttctccgacacccgttgggcggctgatattcatatcaccccggatggtcgccatttatacgcctgcgaccgtaccgccagcctgattaccgttttcagcgtttcggaagatggcagcgtgttgagtaaagaaggcttccagccaacggaaacccagccgcgcggcttcaatgttgatcacagcggcaagtatctgattgccgccgggcaaaaatctcaccacatctcggtatacgaaattgttggcgagcaggggctactgcatgaaaaaggccgctatgcggtcgggcagggaccaatgtgggtggtggttaacgcacactaa

SEQ ID NO: 3 - DNA, 1407 bp, Escherichia coli
atgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 4 - DNA, 1992 bp, Escherichia coli
atgtcctcacgtaaagagcttgccaatgctattcgtgcgctgagcatggacgcagtacagaaagccaaatccggtcacccgggtgcccctatgggtatggctgacattgccgaagtcctgtggcgtgatttcctgaaacacaacccgcagaatccgtcctgggctgaccgtgaccgcttcgtgctgtccaacggccacggctccatgctgatctacagcctgctgcacctcaccggttacgatctgccgatggaagaactgaaaaacttccgtcagctgcactctaaaactccgggtcacccggaagtgggttacaccgctggtgtggaaaccaccaccggtccgctgggtcagggtattgccaacgcagtcggtatggcgattgcagaaaaaacgctggcggcgcagtttaaccgtccgggccacgacattgtcgaccactacacctacgccttcatgggcgacggctgcatgatggaaggcatctcccacgaagtttgctctctggcgggtacgctgaagctgggtaaactgattgcattctacgatgacaacggtatttctatcgatggtcacgttgaaggctggttcaccgacgacaccgcaatgcgtttcgaagcttacggctggcacgttattcgcgacatcgacggtcatgacgcggcatctatcaaacgcgcagtagaagaagcgcgcgcagtgactgacaaaccttccctgctgatgtgcaaaaccatcatcggtttcggttccccgaacaaagccggtacccacgactcccacggtgcgccgctgggcgacgctgaaattgccctgacccgcgaacaactgggctggaaatatgcgccgttcgaaatcccgtctgaaatctatgctcagtgggatgcgaaagaagcaggccaggcgaaagaatccgcatggaacgagaaattcgctgcttacgcgaaagcttatccgcaggaagccgctgaatttacccgccgtatgaaaggcgaaatgccgtctgacttcgacgctaaagcgaaagagttcatcgctaaactgcaggctaatccggcgaaaatcgccagccgtaaagcgtctcagaatgctatcgaagcgttcggtccgctgttgccggaattcctcggcggttctgctgacctggcgccgtctaacctgaccctgtggtctggttctaaagcaatcaacgaagatgctgcgggtaactacatccactacggtgttcgcgagttcggtatgaccgcgattgctaacggtatctccctgcacggtggcttcctgccgtacacctccaccttcctgatgttcgtggaatacgcacgtaacgccgtacgtatggctgcgctgatgaaacagcgtcaggtgatggtttacacccacgactccatcggtctgggcgaagacggcccgactcaccagccggttgagcaggtcgcttctctgcgcgtaaccccgaacatgtctacatggcgtccgtgtgaccaggttgaatccgcggtcgcgtggaaatacggtgttgagcgtcaggacggcccgaccgcactgatcctctcccgtcagaacctggcgcagcaggaacgaactgaagagcaactggcaaacatcgcgcgcggtggttatgtgctgaaagactgcgccggtcagccggaactgattttcatcgctaccggttcagaagttgaactggctgttgctgcctacgaaaaactgactgccgaaggcgtgaaagcgcgcgtggtgtccatgccgtctaccgacgcatttgacaagcaggatgctgcttaccgtgaatccgtactgccgaaagcggttactgcacgcgttgctgtagaagcgggtattgctgactactggtacaagtatgttggcctgaacggtgctatcgtcggtatgaccaccttcggtgaatctgctccggcagagctgctgtttgaagagttcggcttcactgttgataacgttgttgcgaaagcaaaagaactgctgtaa

SEQ ID NO: 5 - DNA, 1557 bp, Escherichia coli
ttgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttacgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctga

SEQ ID NO: 6 - DNA, 865 bp, Escherichia coli
aaggagatatacataacttcactatatggagatgggcgatctgtatctgatcaatggtgaagcccgcgcccatacccgcacgctgaacgtgaagcagaactacgaagagtggttttcgttcgtcggtgaacaggatctgccgctggccgatctcgatgtgatcctgatgcgtaaagacccgccgtttgataccgagtttatctacgcgacctatattctggaacgtgccgaagagaaagggacgctgatcgttaacaagccgcagagcctgcgcgactgtaacgagaaactgtttaccgcctggttctctgacttaacgccagaaacgctggttacgcgcaataaagcgcagctaaaagcgttctgggagaaacacagcgacatcattcttaagccgctggacggtatgggcggcgcgtcgattttccgcgtgaaagaaggcgatccaaacctcggcgtgattgccgaaaccctgactgagcatggcactcgctactgcatggcgcaaaattacctgccagccattaaagatggcgacaaacgcgtgctggtggtggatggcgagccggtaccgtactgcctggcgcgtattccgcaggggggcgaaacccgtggcaatctggctgccggtggtcgcggtgaacctcgtccgctgacggaaagtgactggaaaatcgcccgtcagatcgggccgacgctgaaagaaaaagggctgatttttgttggtctggatatcatcggcgaccgtctgactgaaattaacgtcaccagcccaacctgtattcgtgagattgaagcagagtttccggtgtcgatcaccggaatgttaatggatgccatcgaagcacgtttacagcagcagtaa

SEQ ID NO: 7 - DNA, 1353 bp, Escherichia coli
atgactaaacactatgattacatcgccatcggcggcggcagcggcggtatcgcctccatcaaccgcgcggctatgtacggccagaaatgtgcgctgattgaagccaaagagctgggcggcacctgcgtaaatgttggctgtgtgccgaaaaaagtgatgtggcacgcggcgcaaatccgtgaagcgatccatatgtacggcccggattatggttttgataccactatcaataaattcaactgggaaacgttgatcgccagccgtaccgcctatatcgaccgtattcatacttcctatgaaaacgtgctcggtaaaaataacgttgatgtaatcaaaggctttgcccgcttcgttgatgccaaaacgctggaggtaaacggcgaaaccatcacggccgatcatattctgatcgccacaggcggtcgtccgagccacccggatattccgggcgtggaatacggtattgattctgatggcttcttcgcccttcctgctttgccagagcgcgtggcggttgttggcgcgggttacatcgccgttgagctggcgggcgtgattaacggcctcggcgcgaaaacgcatctgtttgtgcgtaaacatgcgccgctgcgcagcttcgacccgatgatttccgaaacgctggtcgaagtgatgaacgccgaaggcccgcagctgcacaccaacgccatcccgaaagcggtagtgaaaaataccgatggtagcctgacgctggagctggaagatggtcgcagtgaaacggtggattgcctgatttgggcgattggtcgcgagcctgccaatgacaacatcaacctggaagccgctggcgttaaaactaacgaaaaaggctatatcgtcgtcgataaatatcaaaacaccaatattgaaggtatttacgcggtgggcgataacacgggtgcagtggagctgacaccggtggcagttgcagcgggtcgccgtctctctgaacgcctgtttaataacaagccggatgagcatctggattacagcaacattccgaccgtggtcttcagccatccgccgattggtactgttggtttaacggaaccgcaggcgcgcgagcagtatggcgacgatcaggtgaaagtgtataaatcctctttcaccgcgatgtataccgccgtcaccactcaccgccagccgtgccgcatgaagctggtgtgcgttggatcggaagagaagattgtcggtattcacggcattggctttggtatggacgaaatgttgcagggcttcgcggtggcgctgaagatgggggcaaccaaaaaagacttcgacaataccgtcgccattcacccaacggcggcagaagagttcgtgacaatgcgttaa

SEQ ID NO: 8 - DNA, 1098 bp, Escherichia coli
atgagcattgagattgccaatattaagaagtcgtttggtcgcacccaggtgctgaacgatatctcactggatattccttcaggtcagatggtcgcgttgctggggccgtccggttccgggaaaaccacgctgctgcgcattatcgccgggctggagcatcaaaccagcgggcatattcgcttccacggcaccgacgtgagccgcctgcacgcacgtgatcgtaaagtcggtttcgtgttccagcattacgcgctgttccgccatatgacggtgttcgacaatatcgcttttggcctgacggtgctgccgcgtcgcgagcgcccgaatgccgcagccatcaaagcgaaagtgacaaaattgctggaaatggtccagcttgcccatctggcggatcgttatccggcgcagctttccggcggccagaaacagcgcgtggcgctggcgcgcgcgctggctgtggaaccgcaaattctgctgcttgatgaaccgtttggcgcgctggatgcgcaggtgcgtaaagagctgcgtcgctggctgcgtcaactccatgaagaactaaaattcaccagcgtttttgtgacccacgatcaggaagaagcgaccgaagtagctgatcgtgtagttgtgatgagccagggcaatattgaacaggctgacgcgccggatcaggtatggcgcgaaccggcgacccgttttgtgctcgaatttatgggcgaagtgaaccgcctgcagggaaccattcgcggcgggcagttccatgttggcgcgcatcgctggccgctgggctacacacctgcgtatcaggggccggtggatctcttcctgcgcccttgggaagtggatatcagccgccgtaccagcctcgattcgccgctgccggtacaggtactggaagccagcccgaaaggtcactacacccaattagtggtgcagccgctggggtggtacaacgaaccgctgacggtcgtgatgcatggcgacgatgccccgcagcgtggcgagcgtttattcgttggtctgcaacatgcgcggctgtataacggcgacgagcgtatcgaaacccgcgatgaggaacttgctctcgcacaaagcgcctga

SEQ ID NO: 9 - DNA, 834 bp, Escherichia coli
atgtttgctgtctcctccagacgcgtgctgccgggctttaccttaagcctcggcaccagtctgctgtttgtgtgcctgattttgctgctgccgctctccgcgctggtgatgcaactggcccagatgagctgggcgcagtactgggaggtgatcaccaacccgcaggtggtcgcggcctacaaagtaacgctgctgtcggcgtttgtggcatcgatttttaacggcgttttcggtctgctgatggcgtggatcctaacccgctatcgcttcccaggccgcacgctgcttgatgcgctgatggatttaccctttgcgctgccaacggctgtcgccggtttaacgctggcctcgctcttttccgtaaacggtttttacggtgaatggctggcgaagtttgatatcaaagtcacctatacatggctggggattgcggtggctatggcctttaccagcattccgtttgtggtgcgtaccgtgcagccggtgctggaagagttaggcccggaatatgaagaagcggcggaaacgcttggtgcaacgcgctggcagagtttctgcaaagtggtgctgccggagctttctccggcgctggtggcgggcgtggcgctgtcgtttacccgtagtcttggtgaatttggcgcggtgatttttatcgccggaaatatcgcgtggaagacggaagtgacgtcgctgatgatttttgtgcgcttacaggagtttgattacccggcagcgagcgcgattgcttcggtgatcctcgcggcatctctgctgctgctgttctcaattaacactctgcaaagtcgctttggtcggcgtgtggtaggtcattaa

SEQ ID NO: 10 - DNA, 876 bp, Escherichia coli
atggcggaagttacccaattgaagcgttatgacgcgcgcccgattaactggggcaaatggtttctgattggcatcgggatgctggtttcggcgttcatcctgctggtgccgatgatttacatcttcgtgcaggcattcagcaaggggctgatgccggttttacagaatctggccgatccggacatgctgcacgccatctggctgacggtgatgatcgcgctgattgccgtaccggtaaacctggtgttcggcattctgctggcctggctggtgacgcgctttaacttccctggacgccagttactgctgacgctactggacattccgtttgccgtatcgccggtggttgccggtctggtgtatttgctgttctacggctctaacggcccgctcggcggttggctcgacgagcataacctgcaaattatgttctcctggccgggaatggtgctggtcaccatcttcgtgacgtgtccgtttgtggtgcgcgaactggtgccggtgatgttaagccagggcagccaggaagacgaagcggcgattttgcttggcgcgtccggctggcagatgttccgtcgcgtcacattaccgaacatccgctgggcgctgctttatggcgtggtgttgaccaacgcccgcgcaattggcgagtttggcgcggtgtcggtggtttccggctcgattcgcggcgaaaccctgtcgctgccgttacagattgaattgctggagcaggactacaacaccgtcggctcctttaccgctgcggcgctgttaacgctgatggcgattatcaccctgtttttaaaaagtatgttgcagtggcgcctggagaatcaggaaaaacgcgcacagcaggaggaacatcatgagcattga

SEQ ID NO: 11 - DNA, 1017 bp, Escherichia coli
atggccgttaacttactgaaaaagaactcactcgcgctggtcgcttctctgctgctggcgggccatgtacaggcaacggaactgctgaacagttcttatgacgtctcccgcgagctgtttgccgccctgaatccgccgtttgagcaacaatgggcaaaagataacggcggcgacaaactgacgataaaacaatctcatgccgggtcatcaaaacaggcgctggcgattttacagggcttaaaagccgacgttgtcacttataaccaggtgaccgacgtacaaatcctgcacgataaaggcaagctgatcccggccgactggcagtcgcgcctgccgaataatagctcgccgttctactccaccatgggcttcctggtgcgtaagggtaacccgaagaatatccacgattggaacgacctggtgcgctccgacgtgaagctgattttcccgaacccgaaaacgtcgggtaacgcgcgttatacctatctggcggcatggggcgcagcggataaagctgacggtggtgacaaaggcaaaaccgaacagtttatgacccagttcctgaaaaacgttgaagtgttcgatactggcggtcgtggcgcgaccaccacttttgccgagcgcggcctgggcgatgtgctgattagcttcgaatcggaagtgaacaacatccgtaaacagtatgaagcgcagggctttgaagtggtgattccgaaaaccaacattctggcggaattcccggtggcgtgggttgataaaaacgtgcaggccaacggtacggaaaaagccgccaaagcctatctgaactggctctatagcccgcaggcgcaaaccatcatcaccgactattactaccgcgtgaataacccggaggtgatggacaaactgaaagacaaattcccgcagaccgagctgttccgcgtggaagacaaatttggctcctggccggaagtgatgaaaacccacttcaccagcggcggcgagttagacaagctgttagcggcggggcgtaactga

SEQ ID NO: 12 - DNA, 990 bp, Escherichia coli
atgaacaagtggggcgtagggttaacatttttgctggcggcaaccagcgttatggcaaaggatattcagcttcttaacgtttcatatgatccaacgcgcgaattgtacgaacagtacaacaaggcattcagcgcccactggaaacagcaaactggtgataacgtggtgattcgtcagtcacacggtggctcaggtaaacaagcgacgtcggtaatcaacggtattgaagctgatgttgtcacgctggctctggcctatgacgtggacgcaattgcggaacgcgggcggattgataaagagtggatcaaacgtctgccggataactccgcaccgtacacttccaccattgttttcctggtacgtaagggaaatccgaagcagatccatgactggaacgatctgattaaaccgggtgtttcggtgatcacgcctaatccgaaaagctctggtggcgcgcgctggaactacctggcagcctggggctacgcgctgcatcacaacaacaacgatcaggcaaaagcacaggattttgttcgggcactgtataaaaacgtcgaagttctggattctggcgcgcgtggctccactaacacttttgtcgagcgcggaattggcgatgtactgattgcctgggaaaacgaagctctgctggcagcgaatgaactggggaaagataaattcgaaatcgtcacgccgagtgagtctatcctcgcagagccaaccgtgtcggtggtcgataaagtggtcgagaaaaaaggtactaaagaggtggcggaagcctacctgaaatatctctactcgccagaaggtcaggaaattgccgcgaaaaactactaccgtccgcgcgacgctgaggtggcgaaaaagtacgaaaatgcgtttccaaagctgaagttattcaccattgatgaagagttcggcggctggacgaaagcgcaaaaagagcattttgctaacggcggtacgttcgatcagatcagcaaacgctga

SEQ ID NO: 13 - DNA, 963 bp, Escherichia coli
atggcaatttcatcgcgtaacacacttcttgccgcactggcattcatcgcttttcaggcacaggcggtgaacgtcaccgtggcgtatcaaacctcagccgaaccggcgaaagtggctcaggccgacaacacctttgctaaagaaagcggagcaaccgtggactggcgtaagtttgacagcggagccagcatcgtgcgggcgctggcttcaggcgacgtgcaaatcggcaacctcggttccagcccgttagcggttgcagccagccaacaggtgccgattgaagtcttcttgctggcgtcaaaactgggtaactccgaagcgctggtggtaaagaaaactatcagcaaaccggaagatctgattggcaaacgcatcgccgtaccgtttatctccaccacccactacagcctgctggcggcactgaaacactggggcattaaacccgggcaagtggagattgtgaacctgcagccgcccgcgattatcgctgcctggcagcggggagatattgatggtgcttatgtctgggcaccggcggttaacgccctggaaaaagacggcaaggtgttgaccgattctgaacaggtcgggcagtggggcgcgccaacgctggacgtctgggtggtgcgcaaagattttgccgagaaacatcctgaggtcgtgaaagcgttcgctaaaagcgccatcgatgctcagcaaccgtacattgctaacccagacgtgtggctgaaacagccggaaaacatcagcaaactggcgcgtttaagcggcgtgcctgaaggtgacgttccggggctggtgaaggggaatacctatctgacgccgcagcaacaaacggcagaactgaccggaccggtgaacaaagcgatcatcgacaccgcgcagtttttgaaagagcagggcaaggtcccggctgtagcgaatgattacagccagtacgttacctcgcgcttcgtgcaataa

SEQ ID NO: 14 - DNA, 768 bp, Escherichia coli
atgctgcaaatctctcatctttacgccgattatggcggcaaaccggcactggaagatatcaacctgacgctggaaagcggcgagctactggtggtgctggggccgtccggctgcggtaaaaccaccctgctgaatctgattgccggttttgtgccttatcagcatggcagcattcaactggcgggtaagcgtattgagggaccgggagcagagcgtggcgtagtttttcagaatgaagggctactaccgtggcgcaatgtacaggacaacgtggcgttcggcctgcaattggcaggtatagagaaaatgcagcgactggaaatcgcgcaccagatgctgaaaaaagtggggctggaaggcgcagaaaaacgctacatctggcagctttccggtggtcaacgtcagcgggtggggattgctcgtgcgctggcggcgaatccccagctgttattactcgacgaaccgtttggtgcgctggacgccttcacccgcgaccagatgcaaaccctgctgctgaaactctggcaggagacgggcaagcaggtgctgttgattacccacgatatagaagaagcggtgtttatggcgactgaactggttctgctttcatccggccctggccgtgtgctggagcggctgccgctcaactttgctcgccgctttgttgcgggagagtcgagccgcagcatcaagtccgatccacaattcatcgccatgcgcgaatatgttttaagccgcgtatttgagcaacgggaggcgttctcatga

SEQ ID NO: 15 - DNA, 828 bp, Escherichia coli
atgagtgtgctcattaatgaaaaactgcattcgcggcggctgaaatggcgctggccgctctcgcgtcaggtgaccttaagcattggcacgttagcggttttactcaccgtatggtggacggtggcgacgctgcaactgattagcccgctatttttgccgccgccgcaacaggtactggaaaaactactcaccattgccggaccgcaaggctttatggacgccacgctgtggcagcatctggcagccagtctgacgcgcattatgctggcgctatttgcagcggtgttgttcggtattccggtcgggatcgcgatgggacttagccctacggtacgcggcattctggatccgataatcgagctttatcgtccggtgccgccgctggcttatttgccgctgatggtgatctggtttggtattggtgaaacctcgaagatcttactgatctatttagcgatttttgcaccggtggcgatgtcggcgctggcgggggtgaaaagcgtgcagcaggttcgcattcgtgccgcccagtcgctgggtgccagccgtgcgcaggtgctgtggtttgtcattttgcccggtgcgctgccggaaatcctcaccggattacgtattggtctgggggtgggctggtctacgctggtggcggcggagctgattgccgcgacgcgcggtttaggatttatggttcagtcagcgggtgaatttctcgcaactgacgtggtgctggcggggatcgcggtgattgcgattatcgcctttcttttagaactgggtctgcgcgcgttacagcgccgcctgacgccctggcatggagaagtacaatga

SEQ ID NO: 16 - DNA, 801 bp, Escherichia coli
atgaaattagcacatctgggacgtcaggcattgatgggtgtgatggccgtggcgctggttgcgggcatgagcgttaaaagttttgcagatgaaggtctgcttaataaagttaaagagcgcggcacgctgctggtagggctggaaggaacttatccgccgttcagttttcagggagatgacggcaaattaaccggttttgaagtggaatttgcccaacagctggcaaaacatcttggcgttgaggcgtcactaaaaccgaccaaatgggacggtatgctggcgtcgctggactctaaacgtattgatgtggtgattaatcaggtcaccatttctgatgagcgcaagaaaaaatacgatttctcaaccccgtacaccatttctggtattcaggcgctggtgaaaaaaggtaacgaaggcaccattaaaacagccgatgatctgaaaggcaaaaaagtgggggtcggtctgggcaccaactatgaagagtggctgcggcagaatgttcagggcgtcgatgtgcgtacctatgatgatgacccgaccaaatatcaggatctgcgcgtagggcgtatcgatgcgatcctcgttgatcgtctggcggcgctggatctggtgaagaaaaccaacgatacgctggcagtaaccggtgaagcattctcccgtcaggagtctggcgtggcgctgcgtaaaggaaatgaggacctgctgaaagcagtgaatgatgcaattgcggaaatgcaaaaagatggcactctgcaagccctttccgaaaaatggtttggtgctgatgtgaccaaataa

SEQ ID NO: 17 - DNA, 909 bp, Escherichia coli
atggatcaaatacgacttactcacctgcggcaactggaggcggaaagcatccacattattcgcgaggtggcggcagaattctcaaatccggtgatgctctactctatcggtaaagattccagcgtcatgctgcatctggcgcgcaaggcgttttatccaggtacgctgcctttcccgttgctgcatgtcgataccggctggaaattccgcgagatgtatgagttccgcgatcgtactgctaaagcctacggctgcgaactgctggtgcataaaaacccggaaggcgtggcgatggggattaatccattcgtgcacggcagcgcgaaacataccgatattatgaaaactgaaggcctgaaacaggcgctgaacaaatacggttttgatgccgccttcggtggtgcgcgccgtgacgaagagaaatcccgcgctaaagagcgaatttactctttccgtgaccgcttccatcgctgggatccgaaaaatcagcgcccggagctgtggcacaactacaacgggcaaattaacaaaggcgaaagcatccgcgtcttcccgctctctaactggaccgagcaggatatctggcaatacatctggctggaaaatatcgacattgttccgctatatctcgctgcggaacgtccggttctggaacgcgacggtatgttgatgatgattgatgacaaccgtatcgacctgcaaccgggcgaagtgattaaaaaacggatggtgcgtttccgtacgctgggctgctggccgctgaccggtgcggtggagtcaaatgcacaaacactgccggaaatcatcgaagagatgctggtttccaccaccagtgaacgtcagggccgcgtgattgaccgcgaccaggcggggtctatggagctgaaaaaacgtcaggggtatttttaa

SEQ ID NO: 18 - DNA, 1428 bp, Escherichia coli
atgaacaccgcacttgcacaacaaatcgccaatgaaggcggcgtcgaagcctggatgattgcgcaacaacataaaagcctgctgcgttttctgacctgtggtagcgtcgatgacggcaaaagtactctgattggtcgtctgctgcacgatacccgccaaatctacgaagatcagctctcatcgctgcataacgacagtaagcgtcacggcacccagggcgaaaagctggatctggctctgctggtggacggcctgcaagctgagcgcgaacagggcatcaccattgacgtggcctaccgctatttctctaccgagaagcgtaaatttattatcgccgacaccccagggcacgagcagtacacccgcaatatggcgactggcgcatcgacatgtgaactggcgatcttactgatcgatgcccgtaaaggcgtgctcgatcaaacccgtcgtcacagttttatctccacactgttggggatcaaacatctggtcgtggcgatcaacaaaatggatctggtggattacagtgaagagacgttcacccgtattcgtgaagattatttgacctttgccgggcagctgccgggtaatctggatatccgctttgtgccgctctctgcactggaaggcgacaacgtggcatcgcaaagtgaaagtatgccgtggtacagcggtccgacactgctcgaagtgctggaaaccgtggagatccagcgagtggtggatgctcagccaatgcgcttcccggtgcagtacgttaatcgcccgaatctcgattttcgtggttacgccggaacgctggcatccggtcgcgtggaagtcgggcaacgtgtcaaagtgctgccctctggtgtggaatcaaacgtcgcgcggatcgtgacttttgatggtgatcgcgaagaagcctttgccggagaagcgatcaccctggtgctgacggatgagatcgacatcagccgtggcgatctgctgctggcggcagacgaagcgttaccggcggtgcagagcgcgtcggtggatgtggtatggatggcggaacagccgctttctccagggcagagttacgacatcaaaattgccggtaagaagacgcgcgcgcgtgttgatggcattcgctatcaggttgatattaataaccttacccagcgtgaagttgaaaacctgccactgaatgggatcggcctcgtggatctcacttttgacgagccgctggtgttagatcgttatcaacaaaatccggtgacgggtgggctgatttttatcgatcgcctgagcaatgtgaccgtgggtgccggtatggtgcacgagccagttagccaggcaactgctgcgccatctgaattcagtgcattcgaactggaattgaatgctctggttcgtcgccactttccgcactggggcgcgcgcgatttgctgggggataaataa

SEQ ID NO: 19 - DNA, 1257 bp, Escherichia coli
atgacccttttagcactcggtatcaaccataaaacggcacctgtatcgctgcgagaacgtgtatcgttttcgccggataagctcgatcaggcgcttgacagcctgcttgcgcagccgatggtgcagggcggcgtggtgctgtcgacgtgcaaccgcacggaactttatcttagcgttgaagagcaggacaacctgcaagaggcgttaatccgctggctttgcgattatcacaatcttaatgaagaagatctgcgtaaaagcctctactggcatcaggataacgacgcggttagccatttaatgcgtgttgccagcggcctggattcactggttctgggggagccgcagatcctcggtcaggttaaaaaagcgtttgccgattcgcaaaaaggtcatatgaaggccagcgaactggaacgcatgttccagaaatctttctctgtcgcgaaacgcgttcgcactgaaacagatatcggtgccagcgctgtgtctgtcgcttttgcggcttgtacgctggcgcggcagatctttgaatcgctctctacggtcacagtgttgctggtaggcgcgggcgaaactatcgagctggtggcgcgtcatctgcgcgaacacaaagtacagaagatgattatcgccaaccgcactcgcgaacgtgcccaaattctggcagatgaagtcggcgcggaagtgattgccctgagtgatatcgacgaacgtctgcgcgaagccgatatcatcatcagttccaccgccagcccgttaccgattatcgggaaaggcatggtggagcgcgcattaaaaagccgtcgcaaccaaccaatgctgttggtggatattgccgttccgcgcgatgttgagccggaagttggcaaactggcgaatgcttatctttatagcgttgatgatctgcaaagcatcatttcgcacaacctggcgcagcgtaaagccgcagcggttgaggcggaaactattgtcgctcaggaaaccagcgaatttatggcgtggctgcgagcacaaagcgccagcgaaaccattcgcgagtatcgcagccaggcagagcaagttcgcgatgagttaaccgccaaagcgttagcggcccttgagcagggcggcgacgcgcaagccattatgcaggatctggcatggaaactgactaaccgcttgatccatgcgccaacgaaatcacttcaacaggccgcccgtgacggggataacgaacgcctgaatattctgcgcgacagcctcgggctggagtag

SEQ ID NO: 20 - DNA, 1206 bp, Rhodobacter capsulatus
atggactacaatctcgcgctcgacaaagcgatccagaaactccacgacgagggacgttaccgcacgttcatcgacatcgaacgcgagaagggcgccttccccaaggcgcagtggaaccgccccgatggcggcaagcaggacatcaccgtctggtgcggcaacgactatctgggcatgggccagcacccggtcgttctggccgcgatgcatgaggcgctggaagcggtcggggccggttcgggcggcacccgcaacatctcgggcaccacggcctatcaccgccgtctggaagccgagatcgccgatctgcacggcaaggaagcggcgcttgtcttctcctcggcctatatcgccaatgacgcgacgctctcgacgctgcggctgcttttccccggcctgatcatctattccgacagcctgaaccacgcctcgatgatcgaggggatcaagcgcaatgccgggccgaagcggatcttccgtcacaatgacgtcgcccatctgcgcgagctgatcgccgctgatgatccggccgcgccgaagctgatcgccttcgaatcggtctattcgatggatggcgacttcggcccgatcaaggaaatctgcgacatcgccgatgaattcggcgcgctgacctatatcgacgaagtccatgccgtcggcatgtatggcccccgcggcgcgggcgtggccgagcgtgacggtctgatgcaccgcatcgacatcttcaacggcacgctggcgaaagcctatggcgtcttcggcggctacatcgccgcttcggcgaagatggtcgatgccgtgcgctcctatgcgccgggcttcatcttctcgacctcgctgccgccggcgatcgccgctggcgcgcaggcctcgatcgcgtttttgaaaaccgccgaagggcagaagctgcgcgacgcgcaacagatgcacgcgaaggtgctgaaaatgcggctcaaggcgctggggatgccgatcatcgaccatggcagccacatcgttccggtggtcatcggtgaccccgtgcacaccaaggcggtgtcggacatgctcctgtcggattacggcgtttacgtgcagccgatcaacttcccgacggtgccgcgcggcaccgaacggctgcgcttcaccccctcgccggtgcatgacctgaaacagatcgacgggctggttcatgccatggatctgctctgggcgcgctgtgcgtga

SEQ ID NO: 21 - DNA, 546 bp, Escherichia coli
gtgaaaacattaattcttttctcaacaagggacggacaaacgcgcgagattgcctcctacctggcttcggaactgaaagaactggggatccaggcggatgtcgccaatgtgcaccgcattgaagaaccacagtgggaaaactatgaccgtgtggtcattggtgcttctattcgctatggtcactaccattcagcgttccaggaatttgtcaaaaaacatgcgacgcggctgaattcgatgccgagcgccttttactccgtgaatctggtggcgcgcaaaccggagaagcgtactccacagaccaacagctacgcgcgcaagtttctgatgaactcgcaatggcgtcccgatcgctgcgcggtcattgccggggcgctgcgttacccacgttatcgctggtacgaccgttttatgatcaagctgattatgaagatgtcaggcggtgaaacggatacgcgcaaagaagttgtctataccgattgggagcaggtggcgaatttcgcccgagaaatcgcccatttaaccgacaaaccgacgctgaaataa

SEQ ID NO: 22 - DNA, 663 bp, Escherichia coli
atggcttatcgcgaccaacctttaggtgaactggcgctctctattcctcgcgcttcagctctgtttcgtaaatatgatatggattactgctgtggcggtaagcagacgctggcgcgcgcggcggcacgtaaagaactggatgttgaggtcattgaagctgaactggcaaagctcgctgaacaaccgattgagaaagactggcgtagcgccccgctggcagaaatcatcgaccatatcatcgtgcgctaccacgatcgtcaccgcgagcaactgccggagctgattctgcaagcgactaaagtcgagcgcgttcacgccgacaaaccgagcgtgccaaaagggctgacaaaatacctgaccatgctgcatgaagagctttccagccacatgatgaaagaagagcagatcctcttcccgatgatcaaacaaggcatgggcagccaggcaatggggccaatcagcgtaatggaaagcgagcacgatgaagcgggcgaactgctggaagtgattaaacacaccaccaataacgtcacaccgccgccagaagcctgcaccacctggaaagcgatgtataacggcattaatgaactgattgatgacctgatggatcacatcagtctggaaaacaatgtactgttcccacgcgcgctggcgggtgagtga

SEQ ID NO: 23 - DNA, 1191 bp, Escherichia coli
atgcttgacgctcaaaccatcgctacagtaaaagccaccatccctttactggtggaaacggggccaaagttaaccgcccatttctacgaccgtatgtttactcataacccagaactcaaagaaatttttaacatgagtaaccagcgtaatggcgatcaacgtgaagccctgtttaacgctattgccgcctacgccagtaatattgaaaacctgcctgcgctgctgccagcggtagaaaaaatcgcgcagaagcacaccagcttccagatcaaaccggaacagtacaacatcgtcggtgaacacctgttggcaacgctggacgaaatgttcagcccggggcaggaagtgctggacgcgtggggtaaagcctatggtgtactggctaatgtatttatcaatcgcgaggcggaaatctataacgaaaacgccagcaaagccggtggttgggaaggtactcgcgatttccgcattgtggctaaaacaccgcgcagcgcgcttatcaccagcttcgaactggagccggtcgacggtggcgcagtggcagaataccgtccggggcaatatctcggcgtctggctgaagccggaaggtttcccacatcaggaaattcgtcagtactctttgactcgcaaaccggatggcaaaggctatcgtattgcggtgaaacgcgaagagggtgggcaggtatccaactggttgcacaatcacgccaatgttggcgatgtcgtgaaactggtcgctccggcaggtgatttctttatggctgtcgcagatgacacaccagtgacgttaatctctgccggtgttggtcaaacgccaatgctggcaatgctcgacacgctggcaaaagcaggccacacagcacaagtgaactggttccatgcggcagaaaatggcgatgttcacgcctttgccgatgaagttaaggaactggggcagtcactgccgcgctttaccgcgcacacctggtatcgtcagccgagcgaagccgatcgcgctaaaggtcagtttgatagcgaaggtctgatggatttgagcaaactggaaggtgcgttcagcgatccgacaatgcagttctatctctgcggcccggttggcttcatgcagtttaccgcgaaacagttagtggatctgggcgtgaagcaggaaaacattcattacgaatgctttggcccgcataaggtgctgtaa

SEQ ID NO: 24 - DNA, 2181 bp, Escherichia coli
atgagcacgtcagacgatatccataacaccacagccactggcaaatgcccgttccatcagggcggtcacgaccagagtgcgggggcgggcacaaccactcgcgactggtggccaaatcaacttcgtgttgacctgttaaaccaacattctaatcgttctaacccactgggtgaggactttgactaccgcaaagaattcagcaaattagattactacggcctgaaaaaagatctgaaagccctgttgacagaatctcaaccgtggtggccagccgactggggcagttacgccggtctgtttattcgtatggcctggcacggcgcggggacttaccgttcaatcgatggacgcggtggcgcgggtcgtggtcagcaacgttttgcaccgctgaactcctggccggataacgtaagcctcgataaagcgcgtcgcctgttgtggccaatcaaacagaaatatggtcagaaaatctcctgggccgacctgtttatcctcgcgggtaacgtggcgctagaaaactccggcttccgtaccttcggttttggtgccggtcgtgaagacgtctgggaaccggatctggatgttaactggggtgatgaaaaagcctggctgactcaccgtcatccggaagcgctggcgaaagcaccgctgggtgcaaccgagatgggtctgatttacgttaacccggaaggcccggatcacagcggcgaaccgctttctgcggcagcagctatccgcgcgaccttcggcaacatgggcatgaacgacgaagaaaccgtggcgctgattgcgggtggtcatacgctgggtaaaacccacggtgccggtccgacatcaaatgtaggtcctgatccagaagctgcaccgattgaagaacaaggtttaggttgggcgagcacttacggcagcggcgttggcgcagatgccattacctctggtctggaagtagtctggacccagacgccgacccagtggagcaactatttcttcgagaacctgttcaagtatgagtgggtacagacccgcagcccggctggcgcaatccagttcgaagcggtagacgcaccggaaattatcccggatccgtttgatccgtcgaagaaacgtaaaccgacaatgctggtgaccgacctgacgctgcgttttgatcctgagttcgagaagatctctcgtcgtttcctcaacgatccgcaggcgttcaacgaagcctttgcccgtgcctggttcaaactgacgcacagggatatggggccgaaatctcgctacatcgggccggaagtgccgaaagaagatctgatctggcaagatccgctgccgcagccgatctacaacccgaccgagcaggacattatcgatctgaaattcgcgattgcggattctggtctgtctgttagtgagctggtatcggtggcctgggcatctgcttctaccttccgtggtggcgacaaacgcggtggtgccaacggtgcgcgtctggcattaatgccgcagcgcgactgggatgtgaacgccgcagccgttcgtgctctgcctgttctggagaaaatccagaaagagtctggtaaagcctcgctggcggatatcatagtgctggctggtgtggttggtgttgagaaagccgcaagcgccgcaggtttgagcattcatgtaccgtttgcgccgggtcgcgttgatgcgcgtcaggatcagactgacattgagatgtttgagctgctggagccaattgctgacggtttccgtaactatcgcgctcgtctggacgtttccaccaccgagtcactgctgatcgacaaagcacagcaactgacgctgaccgcgccggaaatgactgcgctggtgggcggcatgcgtgtactgggtgccaacttcgatggcagcaaaaacggcgtcttcactgaccgcgttggcgtattgagcaatgacttcttcgtgaacttgctggatatgcgttacgagtggaaagcgaccgacgaatcgaaagagctgttcgaaggccgtgaccgtgaaaccggcgaagtgaaatttacggccagccgtgcggatctggtgtttggttctaactccgtcctgcgtgcggtggcggaagtttacgccagtagcgatgcccacgagaagtttgttaaagacttcgtggcggcatgggtgaaagtgatgaacctcgaccgtttcgacctgctgtaa

SEQ ID NO: 25 - DNA, 2262 bp, Escherichia coli
atgtcgcaacataacgaaaagaacccacatcagcaccagtcaccactacacgattccagcgaagcgaaaccggggatggactcactggcacctgaggacggctctcatcgtccagcggctgaaccaacaccgccaggtgcacaacctaccgccccagggagcctgaaagcccctgatacgcgtaacgaaaaacttaattctctggaagacgtacgcaaaggcagtgaaaattatgcgctgaccactaatcagggcgtgcgcatcgccgacgatcaaaactcactgcgtgccggtagccgtggtccaacgctgctggaagattttattctgcgcgagaaaatcacccactttgaccatgagcgcattccggaacgtattgttcatgcacgcggatcagccgctcacggttatttccagccatataaaagcttaagcgatattaccaaagcggatttcctctcagatccgaacaaaatcaccccagtatttgtacgtttctctaccgttcagggtggtgctggctctgctgataccgtgcgtgatatccgtggctttgccaccaagttctataccgaagagggtatttttgacctcgttggcaataacacgccaatcttctttatccaggatgcgcataaattccccgattttgttcatgcggtaaaaccagaaccgcactgggcaattccacaagggcaaagtgcccacgatactttctgggattatgtttctctgcaacctgaaactctgcacaacgtgatgtgggcgatgtcggatcgcggcatcccccgcagttaccgcaccatggaaggcttcggtattcacaccttccgcctgattaatgccgaagggaaggcaacgtttgtacgtttccactggaaaccactggcaggtaaagcctcactcgtttgggatgaagcacaaaaactcaccggacgtgacccggacttccaccgccgcgagttgtgggaagccattgaagcaggcgattttccggaatacgaactgggcttccagttgattcctgaagaagatgaattcaagttcgacttcgatcttctcgatccaaccaaacttatcccggaagaactggtgcccgttcagcgtgtcggcaaaatggtgctcaatcgcaacccggataacttctttgctgaaaacgaacaggcggctttccatcctgggcatatcgtgccgggactggacttcaccaacgatccgctgttgcagggacgtttgttctcctataccgatacacaaatcagtcgtcttggtgggccgaatttccatgagattccgattaaccgtccgacctgcccttaccataatttccagcgtgacggcatgcatcgcatggggatcgacactaacccggcgaattacgaaccgaactcgattaacgataactggccgcgcgaaacaccgccggggccgaaacgcggcggttttgaatcataccaggagcgcgtggaaggcaataaagttcgcgagcgcagcccatcgtttggcgaatattattcccatccgcgtctgttctggctaagtcagacgccatttgagcagcgccatattgtcgatggtttcagttttgagttaagcaaagtcgttcgtccgtatattcgtgagcgcgttgttgaccagctggcgcatattgatctcactctggcccaggcggtggcgaaaaatctcggtatcgaactgactgacgaccagctgaatatcaccccacctccggacgtcaacggtctgaaaaaggatccatccttaagtttgtacgccattcctgacggtgatgtgaaaggtcgcgtggtagcgattttacttaatgatgaagtgagatcggcagaccttctggccattctcaaggcgctgaaggccaaaggcgttcatgccaaactgctctactcccgaatgggtgaagtgactgcggatgacggtacggtgttgcctatagccgctacctttgccggtgcaccttcgctgacggtcgatgcggtcattgtcccttgcggcaatatcgcggatatcgctgacaacggcgatgccaactactacctgatggaagcctacaaacaccttaaaccgattgcgctggcgggtgacgcgcgcaagtttaaagcaacaatcaagatcgctgaccagggtgaagaagggattgtggaagctgacagcgctgacggtagttttatggatgaactgctaacgctgatggcagcacaccgcgtgtggtcacgcattcctaagattgacaaaattcctgcctga

SEQ ID NO: 26 - DNA, 621 bp, Escherichia coli
atgagctataccctgccatccctgccgtatgcttacgatgccctggaaccgcacttcgataagcagaccatggaaatccaccacaccaaacaccatcagacctacgtaaacaacgccaacgcggcgctggaaagcctgccagaatttgccaacctgccggttgaagagctgatcaccaaactggaccagctgccagcagacaagaaaaccgtactgcgcaacaacgctggcggtcacgctaaccacagcctgttctggaaaggtctgaaaaaaggcaccaccctgcagggtgacctgaaagcggctatcgaacgtgacttcggctccgttgataacttcaaagcagaatttgaaaaagcggcagcttcccgctttggttccggctgggcatggctggtgctgaaaggcgataaactggcggtggtttctactgctaaccaggattctccgctgatgggtgaagctatttctggcgcttccggcttcccgattatgggcctggatgtgtgggaacatgcttactacctgaaattccagaaccgccgtccggactacattaaagagttctggaacgtggtgaactgggacgaagcagcggcacgttttgcggcgaaaaaataa

SEQ ID NO: 27 - DNA, 582 bp, Escherichia coli
atgtcattcgaattacctgcactaccatatgctaaagatgctctggcaccgcacatttctgcggaaaccatcgagtatcactacggcaagcaccatcagacttatgtcactaacctgaacaacctgattaaaggtaccgcgtttgaaggtaaatcactggaagagattattcgcagctctgaaggtggcgtattcaacaacgcagctcaggtctggaaccatactttctactggaactgcctggcaccgaacgccggtggcgaaccgactggaaaagtcgctgaagctatcgccgcatcttttggcagctttgccgatttcaaagcgcagtttactgatgcagcgatcaaaaactttggttctggctggacctggctggtgaaaaacagcgatggcaaactggctatcgtttcaacctctaacgcgggtactccgctgaccaccgatgcgactccgctgctgaccgttgatgtctgggaacacgcttattacatcgactatcgcaatgcacgtcctggctatctggagcacttctgggcgctggtgaactgggaattcgtagcgaaaaatctcgctgcataa

SEQ ID NO: 28 - DNA, 564 bp, Escherichia coli
atgtccttgattaacaccaaaattaaaccttttaaaaaccaggcattcaaaaacggcgaattcatcgaaatcaccgaaaaagataccgaaggccgctggagcgtcttcttcttctacccggctgactttactttcgtatgcccgaccgaactgggtgacgttgctgaccactacgaagaactgcagaaactgggcgtagacgtatacgcagtatctaccgatactcacttcacccacaaagcatggcacagcagctctgaaaccatcgctaaaatcaaatatgcgatgatcggcgacccgactggcgccctgacccgtaacttcgacaacatgcgtgaagatgaaggtctggctgaccgtgcgaccttcgttgttgacccgcagggtatcatccaggcaatcgaagttaccgctgaaggcattggccgtgacgcgtctgacctgctgcgtaaaatcaaagcagcacagtacgtagcttctcacccaggtgaagtttgcccggctaaatggaaagaaggtgaagcaactctggctccgtctctggacctggttggtaaaatctaa

SEQ ID NO: 29 - DNA, 1566 bp, Escherichia coli
atgctcgacacaaatatgaaaactcaactcaaggcttaccttgagaaattgaccaagcctgttgagttaattgccacgctggatgacagcgctaaatcggcagaaatcaaggaactgttggctgaaatcgcagaactgtcagacaaagtcacctttaaagaagataacagcttgccggtgcgtaagccgtctttcctgatcaccaacccaggttccaaccaggggccacgttttgcaggctccccgctgggccacgagttcacctcgctggtactggcgttgctgtggaccggtggtcatccgtcgaaagaagcgcagtctctgctggagcagattcgccatattgacggtgattttgaattcgaaacctattactcgctctcttgccacaactgcccggacgtggtgcaggcgctgaacctgatgagcgtactgaacccgcgcatcaagcacactgcaattgacggcggcaccttccagaacgaaatcaccgatcgcaacgtgatgggcgttccggcagtgttcgtaaacgggaaagagtttggtcagggccgcatgacgttgactgaaatcgttgccaaaattgatactggcgcggaaaaacgtgcggcagaagagctgaacaagcgtgatgcttatgacgtattaatcgtcggttccggcccggcgggtgcagcggcagcaatttactccgcacgtaaaggcatccgtaccggtctgatgggcgaacgttttggtggtcagatcctcgataccgttgatatcgaaaactacatttctgtaccgaagactgaagggcagaagctggcaggcgcactgaaagttcacgttgatgaatacgacgttgatgtgatcgacagccagagcgccagcaaactgatcccagcagcagttgaaggtggtctgcatcagattgaaacagcttctggcgcggtactgaaagcacgcagcattatcgtggcgaccggtgcaaaatggcgcaacatgaacgttccgggcgaagatcagtatcgcaccaaaggcgtgacctactgcccgcactgcgacggcccgctgtttaaaggtaaacgcgtagcggttatcggcggcggtaactccggcgtggaagcggcaattgacctggcgggtatcgttgagcacgtaacgctgctggaatttgcgccagaaatgaaagccgaccaggttctgcaggacaaactgcgcagcctgaaaaacgtcgacattattctgaatgcgcaaaccacggaagtgaaaggcgacggcagcaaagtcgttggtctggaatatcgagatcgtgtcagcggcgatattcacaacatcgaactggccggtattttcgtccagattggtctgctgccgaacaccaactggctcgaaggcgcagtcgaacgtaaccgcatgggcgagattatcattgatgcgaaatgcgaaaccaacgtgaaaggcgtgttcgcagcgggtgactgtacgacggttccgtacaagcagatcatcatcgccactggcgaaggtgccaaagcctctctgagtgcttttgactacctgattcgcaccaaaactgcataa

SEQ ID NO: 30 - DNA, 258 bp, Escherichia coli
atgcaaaccgttatttttggtcgttcgggttgcccttactgtgtgcgtgcaaaagatctggctgagaaattgagcaatgaacgcgatgattttcagtatcagtatgtagatattcgtgcggaagggatcactaaagaagatctacaacaaaaggcaggtaaacccgtagaaaccgtgccgcagatttttgtcgatcagcaacatatcggcggctataccgattttgctgcatgggtgaaagaaaatctggacgcctga

SEQ ID NO: 31 - DNA, 420 bp, Escherichia coli
atgaataccgtttgtacccattgtcaggccatcaatcgcattcccgacgatcggatcgaagatgcggcaaaatgcggacgctgcggtcacgacttgtttgacggagaggtgattaatgcgaccggtgaaacactcgacaaattgctgaaggatgatctacctgtggtgatcgacttctgggcaccgtggtgcggcccctgccgtaatttcgcaccaatttttgaagatgtcgcgcaagagcgtagcggtaaagtgcgctttgtgaaagtgaataccgaagctgaacgtgaattgagcagtcgctttggaattcgtagtataccgacgatcatgattttcaaaaacggtcaggttgtcgacatgcttaatggcgcagtaccgaaagcgccgttcgatagctggctgaacgaatctctttaa

SEQ ID NO: 32 - DNA, 855 bp, Escherichia coli
atgtccgtagaaaatattgtcaacattaacgaatctaacctgcaacaggttcttgaacagtcgatgaccactccggtgctgttctatttttggtctgaacgtagccagcactgtttgcagttaaccccaattctggaaagcctcgcggcgcagtacaacgggcaatttattctggcgaagctggactgcgacgcggagcagatgattgccgcgcagtttggtctgcgtgcgattccgaccgtgtatctgttccagaacgggcaaccggtagatggcttccaggggccgcaaccggaagaggcgatccgcgccctgctggataaagtgctgccgcgcgaagaagagctgaaagcgcagcaggcgatgcaactgatgcaggaaagcaattacaccgatgccctgccattgctgaaagacgcctggcagttgtcgaatcagaacggggagatcggcctgctgctggcagaaacgctgattgcgctgaaccgttctgaagatgcggaagcggtgctgaaaaccattccgttgcaggatcaggacacccgctaccaggggctggtggcgcaaatcgaactgctgaagcaggcggctgatacgccggaaattcaacagttgcaacagcaggtggcggagaatccagaagatgccgcactggcgacgcaactggcgctgcaactgcatcaggttgggcgcaatgaagaggcgctggagttgctgttcgggcatctgcgtaaagatctcaccgccgcagacggtcagacgcgtaaaacgttccaggagatcctcgctgcgctgggtacgggtgatgcactggcgtcgaagtatcgccgccagctgtatgcattgttgtattga

SEQ ID NO: 33 - DNA, 369 bp, Escherichia coli
atggacatgcattcaggaacctttaacccacaagatttcgcctggcaaggcttaacgctgacacccgcagcggcgatacacatccgtgagctggtggcaaagcagccgggtatggtcggcgtgcgcttaggcgtgaagcaaacgggctgcgcgggctttggctatgtgctcgacagtgttagcgagccggacaaagacgatctgctgtttgaacacgacggcgcgaagctgtttgtcccgctgcaagcgatgccgtttattgatggcacggaagtcgatttcgttcgtgaaggacttaatcagatattcaaatttcacaaccctaaagcccagaatgaatgtggctgtggcgaaagctttggggtatag

SEQ ID NO: 34 - DNA, 1488 bp, Escherichia coli
atgtctcgtaatactgaagcaactgacgatgtcaaaacctggaccggcggcccgctgaattataaagaaggattcttcacccagttagccaccgatgagctggcaaaggggataaacgaagaggtggtgcgcgcaatttcggcgaagcgtaatgagccggagtggatgctggagtttcgtctaaacgcctatcgcgcatggctggagatggaagaaccgcactggttgaaagcgcactacgacaagctgaattatcaggattacagctactactcagcaccatcgtgcggtaattgtgacgacacttgcgcgtctgaacctggcgcggtgcagcaaactggcgcgaacgcctttttaagtaaagaggtggaggcggcgtttgagcagttgggcgttcccgtgcgggaaggcaaagaggtggcggtggatgccattttcgactcagtttcggttgccactacttatcgcgaaaaactggcggagcagggaattattttctgttcctttggtgaggcgatccacgatcacccggaactggtgcgtaaatatctcggcaccgtggtgccggggaatgacaacttctttgccgcgcttaatgcggcggtagcctctgatggtacgtttatttatgtgcctaaaggcgtgcgctgcccgatggaactttccacctattttcgcattaacgcagaaaaaaccgggcagtttgagcgcaccattctggtggccgacgaagacagctacgtcagctacattgaaggctgttccgctccggtgcgtgacagctatcagttacacgcggcagtggtggaagtcatcatccataaaaacgccgaggtgaaatattccacggtacaaaactggtttcctggcgataacaacaccggcggtattctcaacttcgtcaccaagcgtgctttgtgcgaaggcgaaaacagcaaaatgtcatggacgcaatcagaaaccgggtcagcgattacgtggaaatatcccagctgcattttgcgcggcgataactccattggtgagttttactcagtggcgctgaccagcggtcatcagcaagcggataccggcaccaagatgatccacatcggtaaaaacaccaaatcgaccattatctcgaaagggatctctgccggacatagtcagaacagttatcgcggcttagtgaaaatcatgccgacggcaaccaatgcgcgcaatttcactcagtgcgactcaatgctgattggcgctaattgtggggcgcataccttcccgtatgttgagtgtcgtaacaatagtgcgcaactggaacacgaggcaacgacatcacgtattggtgaagatcaactgttttactgcctgcaacgcgggatcagcgaagaagacgccatctcgatgattgttaacggtttctgcaaagacgtgttctcggagctgccgttggaatttgccgttgaagcacaaaaactcctcgccatcagtcttgaacacagcgtcggataa

SEQ ID NO: 35 - DNA, 747 bp, Escherichia coli
atgttaagtattaaagatttacacgtcagcgtggaagataaagctatcctgcgcggattaagcctcgacgttcatcccggcgaagttcacgccattatggggccaaacggttcgggcaaaagtaccttatcggcaacgcttgccgggcgagaagattatgaagtgacgggcggcacggttgagttcaaaggcaaagatttgcttgcgctgtcgccggaagatcgcgcgggcgaaggcatctttatggccttccagtatccggtggagattccaggtgtcagtaaccagtttttcctgcaaacggcacttaatgcggtgcgcagctatcgcggccaggaaacgctcgaccgctttgattttcaggatttgatggaagagaaaatcgctctcctgaagatgccggaagatttattaacccgttcggtaaacgttggtttttccggcggcgagaaaaagcgcaacgatattttgcaaatggcggtgctggaaccggagttatgcattcttgatgagtcggactccgggctggatattgacgcattaaaagtggtcgccgatggcgtgaactcgctgcgtgatggcaagcgctcattcatcattgttacgcactaccaacgcattctcgactacatcaagcctgattacgttcatgtgctatatcagggacgaattgtgaaatccggcgatttcacgttggtcaaacaactggaggagcagggttatggctggcttaccgaacagcagtaa

SEQ ID NO: 36 - DNA, 1272 bp, Escherichia coli
atggctggcttaccgaacagcagtaacgcgctgcaacagtggcatcacttgtttgaagctgaagggacaaaacgctccccgcaagcacagcagcatttacaacaattgctgcgtaccggactgccgacacgtaaacatgaaaactggaaatatacgccgctggaagggctgatcaatagccagtttgtcagcattgcgggagagatatccccacagcagcgtgatgccttagcgttaacgttagactccgtgcggctggtgtttgtcgatgggcgttacgtgcccgcactgagcgatgcaactgaaggcagcggatatgaagtgagcattaacgacgaccgtcagggtttacccgacgctattcaggcggaagtgtttctgcatttgacggaaagcctggcacaaagcgtgacgcatatcgccgtgaagcgcggtcaacggccggcaaagccattgctgttaatgcatatcacccagggcgtggcaggtgaagaggtgaacactgcccattaccgacatcatctggatctggcggaaggtgccgaagcaacggtgatcgaacattttgtcagcctgaatgatgctcgtcattttaccggggcacggttcactatcaacgtcgcagcgaatgcccacttgcagcatatcaagctggcgtttgaaaacccgctcagtcaccactttgctcataacgatttgttgctggctgaggatgccaccgcatttagccacagtttcctgctgggtggcgcagtgttacgacacaacaccagtacgcaactcaatggcgaaaacagcacgctgcggatcaatagcctggcgatgccggtgaaaaacgaggtgtgtgatacccgtacctggctggaacacaataaaggtttttgtaacagccgacagttgcacaaaactatcgtcagcgacaaaggccgcgcggtatttaacggtttgatcaacgtcgcgcagcacgccatcaaaacggatggtcagatgaccaacaacaatctgctgatgggcaaactggcggaagtggatacgaaaccgcagctggaaatctatgcagatgatgtgaaatgcagccacggcgcgacggtggggcgtattgatgatgaacagatattctatctgcgctcgcgcgggatcaatcagcaggatgcccagcagatgatcatttacgccttcgctgccgaactgacggaagcactgcgtgatgaggggcttaaacagcaggtgctggcccgaatcggtcaacggctgccaggaggtgcaagatga

SEQ ID NO: 37 - DNA, 1221 bp, Escherichia coli
atgattttttccgtcgacaaagtgcgggccgactttccggtgctttcgcgtgaggtaaacggtttgccgctggcttatctcgacagcgccgccagtgcgcagaaaccgagccaggtgattgacgccgaggccgagttttatcgtcatggctacgcggcggtgcatcgtggtattcataccttaagcgcccaggcgaccgagaaaatggagaacgtgcgcaagcgggcatcgctgtttattaatgcccgttcggcggaagagctggtgttcgtccgcggcacgacggaagggatcaatctggtcgccaatagctggggcaacagcaacgtgcgggcgggcgataacatcatcatcagtcagatggagcaccacgctaacattgttccctggcagatgctttgcgcacgcgttggcgcagagctgcgtgtgatcccgctcaatcccgatggtacgttgcaactggagacgctgcctacgctgtttgatgagaaaactcgcctgctggcaattactcatgtctccaacgtgcttggcacagaaaatccactggcggaaatgatcacgcttgcgcaccagcatggcgcaaaagtgctggtggatggcgctcaggcggtgatgcatcatccggtggatgttcaggcgctggattgcgacttttacgtgttctccgggcataaactgtatggccccaccggaattggcattctttatgtgaaagaagccttgttgcaggagatgccgccgtgggaagggggcggttctatgatcgccaccgtcagcctgagtgaaggcactacctggaccaaagcaccatggcggtttgaagccggtacacccaataccgggggcatcattggtcttggcgcggcgctggagtatgtttcggcgctggggcttaataacatagccgagtatgaacagaatctgatgcattatgcgctatcacagctggaatctgtaccggatctcactctctatggcccacaaaacaggcttggcgttattgcttttaatctcggtaaacaccacgcctatgatgttggcagttttctcgataattacggcattgctgtgcgtaccggacatcactgcgcaatgccattgatggcctattacaacgtccctgcgatgtgtcgggcgtcgctggccatgtataacacccatgaagaagtggatcgtctggtgaccggcctgcaacgtattcaccgtttgctgggataa

SEQ ID NO: 38 - DNA, 417 bp, Escherichia coli
atggctttattgccggataaagaaaagttgctgcgtaattttttacgctgcgccaactgggaagagaaatatctctacattattgagctgggccagcgtctgccagaattacgcgacgaagacagaagtccacaaaatagcattcagggctgtcagagtcaggtgtggattgtcatgcgccagaatgcccagggaattattgaattacagggcgacagcgatgcggcgattgtgaaagggcttattgcggtcgtctttattctctacgatcagatgacgccgcaggatattgtcaatttcgatgtgcgtccgtggtttgaaaaaatggcgctcacccaacatctcaccccatctcgttcacaaggtctggaagcgatgattcgcgcaattcgcgccaaagccgctgcacttagctaa

SEQ ID NO: 39 - DNA, 1215 bp, Escherichia coli
atgaaattaccgatttatctcgactactccgcaaccacgccggtggacccgcgtgttgccgagaaaatgatgcagtttatgacgatggacggaacctttggtaacccggcctcccgttctcaccgtttcggctggcaggctgaagaagcggtagatatcgcccgtaatcagattgccgatctggtcggcgctgatccgcgtgaaatcgtctttacctctggtgcaaccgaatctgacaacctggcgatcaaaggtgcagccaacttttatcagaaaaaaggcaagcacatcatcaccagcaaaaccgaacacaaagcggtactggatacctgccgtcagctggagcgcgaaggttttgaagtcacctacctggcaccgcagcgtaacggcattatcgacctgaaagaacttgaagcagcgatgcgtgacgacaccatcctcgtgtccatcatgcacgtaaataacgaaatcggcgtggtgcaggatatcgcggctatcggcgaaatgtgccgtgctcgtggcattatctatcacgttgatgcaacccagagcgtgggtaaactgcctatcgacctgagccagttgaaagttgacctgatgtctttctccggtcacaaaatctatggcccgaaaggtatcggtgcgctgtatgtacgtcgtaaaccgcgcgtacgcatcgaagcgcaaatgcacggcggcggtcacgagcgcggtatgcgttccggcactctgcctgttcaccagatcgtcggaatgggcgaggcctatcgcatcgcaaaagaagagatggcgaccgagatggaacgtctgcgcggcctgcgtaaccgtctgtggaacggcatcaaagatatcgaagaagtttacctgaacggtgacctggaacacggtgcgccgaacattctcaacgtcagcttcaactacgttgaaggtgagtcgctgattatggcgctgaaagacctcgcagtttcttcaggttccgcctgtacgtcagcaagcctcgaaccgtcctacgtgctgcgcgcgctggggctgaacgacgagctggcacatagctctatccgtttctctttaggtcgttttactactgaagaagagatcgactacaccatcgagttagttcgtaaatccatcggtcgtctgcgtgacctttctccgctgtgggaaatgtacaagcagggcgtggatctgaacagcatcgaatgggctcatcattaa

SEQ ID NO: 40 - DNA, 387 bp, Escherichia coli
atggcttacagcgaaaaagttatcgaccattacgagaatccgcgtaacgtgggttcctttgacaacaacgacgagaacgtcggcagcggcatggtgggggcaccggcctgtggcgacgtgatgaagttgcagattaaagtcaacgatgaaggtatcattgaagacgcgcgttttaaaacttacggctgcggttccgctatcgcttccagctccctggtcaccgaatgggtgaaagggaagtctctcgacgaagcgcaggcgatcaaaaacaccgatattgctgaagaacttgaactgccgccggtgaaaattcactgttctattctggcagaagacgcgatcaaagccgccattgcggactataaaagcaaacgtgaagcaaaataa

SEQ ID NO: 41 - DNA, 1851 bp, Escherichia coli
atggccttattacaaattagtgaacctggtttgagtgctgcgccgcatcagcgtcgtctggcggccggtattgacctgggcacaaccaactcgctggtggcgacagtgcgcagcggtcaggccgaaacgttagccgatcatgaaggccgtcacctgctgccatctgttgttcactatcaacagcaagggcattcggtgggttatgacgcgcgtactaatgcagcgctcgataccgccaacacaattagttctgttaaacgcctgatgggacgctcgctggctgatatccagcaacgctatccgcatctgccttatcaattccaggccagcgaaaacggcctgccgatgattgaaacggcggcggggctgctgaacccggtgcgcgtttctgcggacatcctcaaagcactggcggcgcgggcaactgaagccctggcaggcgagctggatggtgtagttatcaccgttccggcgtactttgacgatgcccagcgtcagggcaccaaagacgcggcgcgtctggcgggccttcacgtcctgcgcttacttaacgaaccgaccgctgcggctatcgcctacgggctggattccggtcaggaaggcgtgatcgccgtttatgacctcggtggcgggacgtttgatatttccattctgcgcttaagtcgcggcgtgtttgaagtgctggcaaccggcggtgattccgcgctcggcggcgatgatttcgaccatctgctggcggattacattcgcgagcaggcgggcattcctgatcgtagcgataaccgcgttcagcgtgaactgctggatgccgccattgcagccaaaatcgcgctgagcgatgcggactccgtgaccgttaacgttgcgggctggcagggcgaaatcagccgtgaacaattcaatgaactgatcgcgccactggtaaaacgaaccttactggcttgtcgtcgcgcgctgaaagacgcgggtgtagaagctgatgaagtgctggaagtggtgatggtgggcggttctactcgcgtgccgctggtgcgtgaacgggtaggcgaatttttcggtcgtccaccgctgacttccatcgacccggataaagtcgtcgctattggcgcggcgattcaggcggatattctggtgggtaacaagccagacagcgaaatgctgttgcttgatgtgatcccactgtcgctgggcctcgaaacgatgggcggcctggtggagaaagtgattccgcgtaataccactattccggtggcccgcgctcaggatttcaccacctttaaagatggtcagacggcgatgtctatccatgtaatgcagggtgagcgcgaactggtgcaggactgccgctcactggcgcgttttgcgctgcgtggtattccggcgctaccggctggcggtgcgcatattcgcgtgacgttccaggtcgatgccgacggtcttttgagcgtgacggcgatggagaaatccaccggcgttgaggcgtctattcaggtcaaaccgtcttacggtctgaccgatagcgaaatcgcttcgatgatcaaagactcaatgagctatgccgagcaggacgtaaaagcccgaatgctggcagaacaaaaagtagaagcggcgcgtgtgctggaaagtctgcacggcgcgctggctgctgatgccgcgctgttaagcgccgcagaacgtcaggtcattgacgatgctgccgctcacctgagtgaagtggcgcagggcgatgatgttgacgccatcgaacaagcgattaaaaacgtagacaaacaaacccaggatttcgccgctcgccgcatggaccagtcggttcgtcgtgcgctgaaaggccattccgtggacgaggtttaa

SEQ ID NO: 42 - DNA, 516 bp, Escherichia coli
atggattacttcaccctctttggcttgcctgcccgctatcaactcgatacccaggcgctgagcctgcgttttcaggatctacaacgtcagtatcatcctgataaattcgccagcggaagccaggcggaacaactcgccgccgtacagcaatctgcaaccattaaccaggcctggcaaacgctgcgtcatccgttaatgcgcgcggaatatttgctttctttgcacggctttgatctcgccagcgagcagcatactgtgcgcgacaccgcgttcctgatggaacagttggagctgcgcgaagagctggacgagatcgaacaggcgaaagatgaagcgcggctggaaagctttatcaaacgtgtgaaaaagatgtttgatacccgccatcagttgatggttgaacagttagacaacgagacgtgggacgcggcggcggataccgtgcgtaagctgcgttttctcgataaactgcgaagcagtgccgaacaactcgaagaaaaactgctcgatttttaa

SEQ ID NO: 43 - DNA, 294 bp, Escherichia coli
atgaatattcgtccattgcatgatcgcgtgatcgtcaagcgtaaagaagttgaaactaaatctgctggcggcatcgttctgaccggctctgcagcggctaaatccacccgcggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaa

SEQ ID NO: 44 - DNA, 1647 bp, Escherichia coli
atggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgggtggcatgggcggcatgatgtaa

SEQ ID NO: 45 - DNA, 1917 bp, Escherichia coli
atgggtaaaataattggtatcgacctgggtactaccaactcttgtgtagcgattatggatggcaccactcctcgcgtgctggagaacgccgaaggcgatcgcaccacgccttctatcattgcctatacccaggatggtgaaactctagttggtcagccggctaaacgtcaggcagtgacgaacccgcaaaacactctgtttgcgattaaacgcctgattggtcgccgcttccaggacgaagaagtacagcgtgatgtttccatcatgccgttcaaaattattgctgctgataacggcgacgcatgggtcgaagttaaaggccagaaaatggcaccgccgcagatttctgctgaagtgctgaaaaaaatgaagaaaaccgctgaagattacctgggtgaaccggtaactgaagctgttatcaccgtaccggcatactttaacgatgctcagcgtcaggcaaccaaagacgcaggccgtatcgctggtctggaagtaaaacgtatcatcaacgaaccgaccgcagctgcgctggcttacggtctggacaaaggcactggcaaccgtactatcgcggtttatgacctgggtggtggtactttcgatatttctattatcgaaatcgacgaagttgacggcgaaaaaaccttcgaagttctggcaaccaacggtgatacccacctggggggtgaagacttcgacagccgtctgatcaactatctggttgaagaattcaagaaagatcagggcattgacctgcgcaacgatccgctggcaatgcagcgcctgaaagaagcggcagaaaaagcgaaaatcgaactgtcttccgctcagcagaccgacgttaacctgccatacatcactgcagacgcgaccggtccgaaacacatgaacatcaaagtgactcgtgcgaaactggaaagcctggttgaagatctggtaaaccgttccattgagccgctgaaagttgcactgcaggacgctggcctgtccgtatctgatatcgacgacgttatcctcgttggtggtcagactcgtatgccaatggttcagaagaaagttgctgagttctttggtaaagagccgcgtaaagacgttaacccggacgaagctgtagcaatcggtgctgctgttcagggtggtgttctgactggtgacgtaaaagacgtactgctgctggacgttaccccgctgtctctgggtatcgaaaccatgggcggtgtgatgacgacgctgatcgcgaaaaacaccactatcccgaccaagcacagccaggtgttctctaccgctgaagacaaccagtctgcggtaaccatccatgtgctgcagggtgaacgtaaacgtgcggctgataacaaatctctgggtcagttcaacctagatggtatcaacccggcaccgcgcggcatgccgcagatcgaagttaccttcgatatcgatgctgacggtatcctgcacgtttccgcgaaagataaaaacagcggtaaagagcagaagatcaccatcaaggcttcttctggtctgaacgaagatgaaatccagaaaatggtacgcgacgcagaagctaacgccgaagctgaccgtaagtttgaagagctggtacagactcgcaaccagggcgaccatctgctgcacagcacccgtaagcaggttgaagaagcaggcgacaaactgccggctgacgacaaaactgctatcgagtctgcgctgactgcactggaaactgctctgaaaggtgaagacaaagccgctatcgaagcgaaaatgcaggaactggcacaggtttcccagaaactgatggaaatcgcccagcagcaacatgcccagcagcagactgccggtgctgatgcttctgcaaacaacgcgaaagatgacgatgttgtcgacgctgaatttgaagaagtcaaagacaaaaaataa

SEQ ID NO: 46 - DNA, 1131 bp, Escherichia coli
atggctaagcaagattattacgagattttaggcgtttccaaaacagcggaagagcgtgaaatcagaaaggcctacaaacgcctggccatgaaataccacccggaccgtaaccagggtgacaaagaggccgaggcgaaatttaaagagatcaaggaagcttatgaagttctgaccgactcgcaaaaacgtgcggcatacgatcagtatggtcatgctgcgtttgagcaaggtggcatgggcggcggcggttttggcggcggcgcagacttcagcgatatttttggtgacgttttcggcgatatttttggcggcggacgtggtcgtcaacgtgcggcgcgcggtgctgatttacgctataacatggagctcaccctcgaagaagctgtacgtggcgtgaccaaagagatccgcattccgactctggaagagtgtgacgtttgccacggtagcggtgcaaaaccaggtacacagccgcagacttgtccgacctgtcatggttctggtcaggtgcagatgcgccagggattcttcgctgtacagcagacctgtccacactgtcagggccgcggtacgctgatcaaagatccgtgcaacaaatgtcatggtcatggtcgtgttgagcgcagcaaaacgctgtccgttaaaatcccggcaggggtggacactggagaccgcatccgtcttgcgggcgaaggtgaagcgggcgagcatggcgcaccggcaggcgatctgtacgttcaggttcaggttaaacagcacccgattttcgagcgtgaaggcaacaacctgtattgcgaagtcccgatcaacttcgctatggcggcgctgggtggcgaaatcgaagtaccgacccttgatggtcgcgtcaaactgaaagtgcctggcgaaacccagaccggtaagctattccgtatgcgcggtaaaggcgtcaagtctgtccgcggtggcgcacagggtgatttgctgtgccgcgttgtcgtcgaaacaccggtaggcctgaacgaaaggcagaaacagctgctgcaagagctgcaagaaagcttcggtggcccaaccggcgagcacaacagcccgcgctcaaagagcttctttgatggtgtgaagaagttttttgacgacctgacccgctaa

SEQ ID NO: 47 - DNA, 594 bp, Escherichia coli
atgagtagtaaagaacagaaaacgcctgaggggcaagccccggaagaaattatcatggatcagcacgaagagattgaggcagttgagccagaagcttctgctgagcaggtggatccgcgcgatgaaaaagttgcgaatctcgaagctcagctggctgaagcccagacccgtgaacgtgacggcattttgcgtgtaaaagccgaaatggaaaacctgcgtcgtcgtactgaactggatattgaaaaagcccacaaattcgcgctggagaaattcatcaacgaattgctgccggtgattgatagcctggatcgtgcgctggaagtggctgataaagctaacccggatatgtctgcgatggttgaaggcattgagctgacgctgaagtcgatgctggatgttgtgcgtaagtttggcgttgaagtgatcgccgaaactaacgtcccactggacccgaatgtgcatcaggccatcgcaatggtggaatctgatgacgttgcgccaggtaacgtactgggcattatgcagaagggttatacgctgaatggtcgtacgattcgtgcggcgatggttactgtagcgaaagcaaaagcttaa

SEQ ID NO: 48 - DNA, 2574 bp, Escherichia coli
atgcgtctggatcgtcttactaataaattccagcttgctcttgccgatgcccaatcacttgcactcgggcacgacaaccaatttatcgaaccacttcatttaatgagcgccctgctgaatcaggaagggggttcggttagtcctttattaacatccgctggcataaatgctggccagttgcgcacagatatcaatcaggcattaaatcgtttaccgcaggttgaaggtactggtggtgatgtccagccatcacaggatctggtgcgcgttcttaatctttgcgacaagctggcgcaaaaacgtggtgataactttatctcgtcagaactgttcgttctggcggcacttgagtctcgcggcacgctggccgacatcctgaaagcagcaggggcgaccaccgccaacattactcaagcgattgaacaaatgcgtggaggtgaaagcgtgaacgatcaaggtgctgaagaccaacgtcaggctttgaaaaaatataccatcgaccttaccgaacgagccgaacagggcaaactcgatccggtgattggtcgtgatgaagaaattcgccgtaccattcaggtgctgcaacgtcgtactaaaaataacccggtactgattggtgaacccggcgtcggtaaaactgccatcgttgaaggtctggcgcagcgtattatcaacggcgaagtgccggaagggttgaaaggccgccgggtactggcgctggatatgggcgcgctggtggctggggcgaaatatcgcggtgagtttgaagaacgtttaaaaggcgtgcttaacgatcttgccaaacaggaaggcaacgtcatcctatttatcgacgaattacataccatggtcggcgcgggtaaagccgatggcgcaatggacgccggaaacatgctgaaaccggcgctggcgcgtggtgaattgcactgcgtaggtgccacgacgcttgacgaatatcgccagtacattgaaaaagatgctgcgctggaacgtcgtttccagaaagtgtttgttgccgagccttctgttgaagataccattgcgattctgcgtggcctgaaagaacgttacgaattgcaccaccatgtgcaaattactgacccggcaattgttgcagcggcgacgttgtctcatcgctacattgctgaccgtcagctgccggataaagccatcgacctgatcgatgaagcagcatccagcattcgtatgcagattgactcaaaaccagaagaactcgaccgactcgatcgtcgtatcatccagctcaaactggaacaacaggcgttaatgaaagagtctgatgaagccagtaaaaaacgtctggatatgctcaacgaagaactgagcgacaaagaacgtcagtactccgagttagaagaagagtggaaagcagagaaggcatcgctttctggtacgcagaccattaaagcggaactggaacaggcgaaaatcgctattgaacaggctcgccgtgtgggggacctggcgcggatgtctgaactgcaatacggcaaaatcccggaactggaaaagcaactggaagccgcaacgcagctcgaaggcaaaactatgcgtctgttgcgtaataaagtgaccgacgccgaaattgctgaagtgctggcgcgttggacggggattccggtttctcgcatgatggaaagcgagcgcgaaaaactgctgcgtatggagcaagaactgcaccatcgcgtaattggtcagaacgaagcggttgatgcggtatctaacgctattcgtcgtagccgtgcggggctggcggatccaaatcgcccgattggttcattcctgttcctcggcccaactggtgtggggaaaacagagctttgtaaggcgctggcgaactttatgtttgatagcgacgaggcgatggtccgtatcgatatgtccgagtttatggagaaacactcggtgtctcgtttggttggtgcgcctccgggatatgtcggttatgaagaaggtggctacctgaccgaagcggtgcgtcgtcgtccgtattccgtcatcctgctggatgaagtggaaaaagcgcatccggatgtcttcaacattctgttgcaggtactggatgatgggcgtctgactgacgggcaagggagaacggtcgacttccgtaatacggtcgtcattatgacctctaacctcggttccgatctgattcaggaacgcttcggtgaactggattatgcgcacatgaaagagctggtgctcggtgtggtaagccataacttccgtccggaattcattaaccgtatcgatgaagtggtggtcttccatccgctgggtgaacagcacattgcctcgattgcgcagattcagttgaaacgtctgtacaaacgtctggaagaacgtggttatgaaatccacatttctgacgaggcgctgaaactgctgagcgagaacggttacgatccggtctatggtgcacgtcctctgaaacgtgcaattcagcagcagatcgaaaacccgctggcacagcaaatactgtctggtgaattggttccgggtaaagtgattcgcctggaagttaatgaagaccggattgtcgccgtccagtaa

SEQ ID NO: 49 - DNA, 414 bp, Escherichia coli
atgcgtaactttgatttatccccgctttaccgttctgctattggatttgaccgtttgtttaaccacttagaaaacaaccagagccagagtaatggcggctaccctccgtataacgttgaactggtagacgaaaaccattaccgcattgctatcgctgtggctggttttgctgagagcgaactggaaattaccgcccaggataatctgctggtggtgaaaggtgctcacgccgacgaacaaaaagagcgcacctatctgtaccagggcatcgctgaacgcaactttgaacgcaaattccagttagctgagaacattcatgttcgtggtgctaacctggtaaatggtttgctgtatatcgatctcgaacgcgtgattccggaagcgaaaaaaccgcgccgtatcgaaatcaactaa

SEQ ID NO: 50 - DNA, 429 bp, Escherichia coli
atgcgtaacttcgatttatccccactgatgcgtcaatggatcggttttgacaaactggccaacgcactgcaaaacgccggtgaaagccagagcttcccgccgtacaacattgagaaaagcgacgataaccactaccgcattacccttgcgctggcaggtttccgtcaggaagatttagagattcaactggaaggtacgcgcctgagcgtaaaaggcacgccggagcagccaaaagaagagaaaaaatggctgcatcaagggcttatgaatcagccatttagcctgagctttacgctggctgaaaatatggaagtctctggcgcaaccttcgtaaacggtttactgcatattgatttaattcgtaatgagcctgaacccatcgcagcgcagcgtatcgctatcagcgaacgtcccgcgttaaatagctaa

SEQ ID NO: 51 - DNA, 1299 bp, Escherichia coli
atgcaagtttcagttgaaaccactcaaggccttggccgccgtgtaacgattactatcgctgctgacagcatcgagaccgctgttaaaagcgagctggtcaacgttgcgaaaaaagtacgtattgacggcttccgcaaaggcaaagtgccaatgaatatcgttgctcagcgttatggcgcgtctgtacgccaggacgttctgggtgacctgatgagccgtaacttcattgacgccatcattaaagaaaaaatcaatccggctggcgcaccgacttatgttccgggcgaatacaagctgggtgaagacttcacttactctgtagagtttgaagtttatccggaagttgaactgcagggtctggaagcgatcgaagttgaaaaaccgatcgttgaagtgaccgacgctgacgttgacggcatgctggatactctgcgtaaacagcaggcgacctggaaagaaaaagacggcgctgttgaagcagaagaccgcgtaaccatcgacttcaccggttctgtagacggcgaagagttcgaaggcggtaaagcgtctgatttcgtactggcgatgggccagggtcgtatgatcccgggctttgaagacggtatcaaaggccacaaagctggcgaagagttcaccatcgacgtgaccttcccggaagaataccacgcagaaaacctgaaaggtaaagcagcgaaattcgctatcaacctgaagaaagttgaagagcgtgaactgccggaactgactgcagaattcatcaaacgtttcggcgttgaagatggttccgtagaaggtctgcgcgctgaagtgcgtaaaaacatggagcgcgagctgaagagcgccatccgtaaccgcgttaagtctcaggcgatcgaaggtctggtaaaagctaacgacatcgacgtaccggctgcgctgatcgacagcgaaatcgacgttctgcgtcgccaggctgcacagcgtttcggtggcaacgaaaaacaagctctggaactgccgcgcgaactgttcgaagaacaggctaaacgccgcgtagttgttggcctgctgctgggcgaagttatccgcaccaacgagctgaaagctgacgaagagcgcgtgaaaggcctgatcgaagagatggcttctgcgtacgaagatccgaaagaagttatcgagttctacagcaaaaacaaagaactgatggacaacatgcgcaatgttgctctggaagaacaggctgttgaagctgtactggcgaaagcgaaagtgactgaaaaagaaaccactttcaacgagctgatgaaccagcaggcgtaa

SEQ ID NO: 52 - DNA, 531 bp, Escherichia coli
gtgacaactatagtaagcgtacgccgtaacggccatgtggtcatcgctggtgatggtcaggccacgttgggcaataccgtaatgaaaggcaacgtgaaaaaggtccgccgtctgtacaacgacaaagtcatcgcgggctttgcgggcggtactgcggatgcttttacgctgttcgaactgtttgaacgtaaactggaaatgcatcagggccatctggtcaaagccgccgttgagctggcaaaagactggcgtaccgatcgcatgctgcgcaaacttgaagcactgctggcagtcgcggatgaaactgcatcgcttatcatcaccggtaacggtgacgtggtgcagccagaaaacgatcttattgctatcggctccggcggcccttacgcccaggctgcggcgcgcgcgctgttagaaaacactgaacttagcgcccgtgaaattgctgaaaaggcgttggatattgcaggcgacatttgcatctataccaaccatttccacaccatcgaagaattaagctacaaagcgtaa

SEQ ID NO: 53 - DNA, 1332 bp, Escherichia coli
atgtctgaaatgaccccacgcgaaatcgtcagcgaactggataagcacatcatcggccaggacaacgccaagcgttctgtggcgattgctctgcgtaaccgctggcgtcgcatgcagctcaacgaagagctgcgccatgaagtgaccccgaaaaatatcctgatgatcggcccgaccggtgtcggtaaaactgaaatcgcccgtcgtctggctaagctggcgaatgcgccgttcatcaaagttgaagcgaccaaattcaccgaagtgggctacgtcggtaaggaagtggattctattattcgcgatctgaccgatgccgccgtgaaaatggtacgcgtccaggctatcgagaaaaaccgttatcgcgctgaagaactggcagaagaacgtattctcgacgtgctgatcccacctgctaaaaacaactggggacagaccgaacagcagcaggaaccgtccgctgctcgtcaggcattccgcaaaaaactgcgtgaaggccagcttgatgacaaagaaatcgagatcgatcttgccgcagcaccgatgggcgttgaaattatggctcctccgggcatggaagagatgaccagccagctgcagtccatgttccagaacctgggcggccagaagcaaaaagcgcgtaagctgaaaatcaaagacgccatgaagctgctgattgaagaagaagcggcgaaactggtgaacccggaagagctgaagcaagacgctatcgacgctgttgagcagcacgggatcgtgtttatcgacgaaatcgacaaaatctgtaagcgcggcgagtcttccggtccggatgtttctcgtgaaggcgttcagcgtgacctgctgccgctggtagaaggttgcaccgtttccaccaaacacgggatggtcaaaactgaccacattctgtttatcgcttctggcgcgttccagattgcgaaaccgtctgacctgatcccggaactgcaaggtcgtctgccaatccgcgttgaactgcaggcgctgaccaccagcgacttcgagcgtattctgaccgagccgaatgcctctatcaccgtgcagtacaaagcactgatggcgactgaaggcgtaaatatcgagtttaccgactccggtattaaacgcatcgcggaagcggcatggcaggtgaacgaatctaccgaaaacatcggtgctcgtcgtttacacactgttctggagcgtttaatggaagagatttcctacgacgccagcgatttaagcggtcaaaatatcactattgacgcagattatgtgagcaaacatctggatgcgttggtggcagatgaagatctgagccgttttatcctataa

SEQ ID NO: 54 - DNA, 1203 bp, Escherichia coli
atggcaattaaattagaaattaaaaatctttataaaatatttggcgagcatccacagcgagcgttcaaatatatcgaacaaggactttcaaaagaacaaattctggaaaaaactgggctatcgcttggcgtaaaagacgccagtctggccattgaagaaggcgagatatttgtcatcatgggattatccggctcgggtaaatccacaatggtacgccttctcaatcgcctgattgaacccacccgcgggcaagtgctgattgatggtgtggatattgccaaaatatccgacgccgaactccgtgaggtgcgcagaaaaaagattgcgatggtcttccagtcctttgccttaatgccgcatatgaccgtgctggacaatactgcgttcggtatggaattggccggaattaatgccgaagaacgccgggaaaaagcccttgatgcactgcgtcaggtcgggctggaaaattatgcccacagctacccggatgaactctctggcgggatgcgtcaacgtgtgggattagcccgcgcgttagcgattaatccggatatattattaatggacgaagccttctcggcgctcgatccattaattcgcaccgagatgcaggatgagctggtaaaattacaggcgaaacatcagcgcaccattgtctttatttcccacgatcttgatgaagccatgcgtattggcgaccgaattgccattatgcaaaatggtgaagtggtacaggtcggcacaccggatgaaattctcaataatccggcgaatgattatgtccgtaccttcttccgtggcgttgatattagtcaggtattcagtgcgaaagatattgcccgccggacaccgaatggcttaattcgtaaaacccctggcttcggcccacgttcggcactgaaattattgcaggatgaagatcgcgaatatggctacgttatcgaacgcggtaataagtttgtcggcgcagtctccatcgattcgcttaaaaccgcgttaacgcagcagcaaggtcttgatgcggcgctgattgatgcgccgttagcagtcgatgcacaaacgcctcttagcgagttgctctctcatgtcggacaggcaccctgtgcggtgcccgtggtcgacgaggaccaacagtatgtcggcatcatttcgaaaggaatgctgctgcgcgctttagatcgtgagggggtaaataatggctga

SEQ ID NO: 55 - DNA, 1065 bp, Escherichia coli
atggctgatcaaaataatccgtgggataccacgccagcggcggacagtgccgcgcaatccgcagacgcctggggtacaccgacgactgcaccgactgacggcggtggtgctgactggctgaccagtacgcctgcgccaaacgtcgagcattttaatattctcgatccgttccataaaacgctgatcccgctcgacagttgggtcactgaagggatcgactgggtcgttacccatttccgtcccgtcttccagggcgtgcgcgttccggttgattatatcctcaacggtttccagcaattgctgctgggtatgcccgcaccggtggcgattatcgttttcgctctcatcgcctggcagatttccggggtcggaatgggtgtggcgacgctggtttcgctgattgccatcggcgcaatcggtgcctggtcgcaggcaatggtgactctggcgctggtgttaaccgccctgctgttctgtatcgtcatcggtttgccgttggggatatggctggcgagaagtccgcgagcggcgaaaattattcgtccactgcttgatgccatgcagaccacgccagcgtttgtttatctggtgccaatcgtcatgctatttggtatcggtaacgtgccgggcgtggtggtgacgatcatctttgctctgccgccgattatccgtctgaccattctggggattaaccaggttccggcggatctgattgaagcctcgcgctcattcggtgccagcccgcgccagatgctgttcaaagttcagttaccgctggcgatgccgaccattatggcgggcgttaaccagacgctgatgctggccctttctatggtggtcatcgcctcgatgattgccgtcggcgggttgggtcagatggtacttcgcggtatcggtcgtctggatatggggcttgccaccgttggcggcgtcgggattgtgatcctcgccattatcctcgatcgtctgacgcaggccgttgggcgcgactcacgcagtcgcggcaaccgtcgctggtacaccactggccctgttggtctgctgacccgcccattcattaagtaa

SEQ ID NO: 56 - DNA, 993 bp, Escherichia coli
atgcgacatagcgtactttttgcgacagcgtttgccacgcttatctctacacaaacttttgctgccgatctgccgggcaaaggcattactgttaatccagttcagagcaccatcactgaagaaaccttccagacgctgctggtcagtcgtgcgctggagaaattaggttataccgtcaacaaacccagcgaagtagattacaacgttggctacacctcgcttgcttccggcgatgcaaccttcaccgccgtgaactggacgccactgcatgacaacatgtacgaagctgccggtggcgataagaaattttatcgtgaaggggtatttgttaacggcgcggcacagggttacctgatcgataagaaaaccgccgaccagtacaaaatcaccaacatcgcacaactgaaagatccgaagatcgccaaactgttcgataccaacggcgacggaaaagcggatttaaccggttgtaaccctggctggggctgcgaaggtgcgatcaaccaccagcttgccgcgtatgaactgaccaacaccgtgacgcataatcaggggaactacgcagcgatgatggccgacaccatcagtcgctacaaagagggcaaaccggtgttttattacacctggacgccgtactgggtgagtaacgaactgaagccgggcaaagatgtcgtctggttgcaggtgccgttctccgcactgccgggcgataaaaacgccgataccaaactgccgaatggtgcgaattatggcttcccggtcagcaccatgcatatcgttgccaacaaagcctgggccgagaaaaacccggcagcagcgaaactgtttgccattatgcagttgccagtggcagatattaacgcccagaacgccattatgcatgacggcaaagcctcagaaggcgatattcagggacacgttgatggttggatcaaagcccaccagcagcagttcgatggctgggtgaatgaggcgctggcagcgcagaagtaa

SEQ ID NO: 57 - DNA, 1425 bp, Escherichia coli
atgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtag

SEQ ID NO: 58 - DNA, 801 bp, Escherichia coli
gtgacagaaccgttaaccgaaacccctgaactatccgcgaaatatgcctggttttttgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaa

SEQ ID NO: 59 - DNA, 1671 bp, Escherichia coli
ttgcaatttgactacatcattattggtgccggctcagccggcaacgttctcgctacccgtctgactgaagatccgaatacctccgtgctgctgcttgaagcgggcggcccggactatcgctttgacttccgcacccagatgcccgctgccctggcattcccgctacagggtaaacgctacaactgggcctatgaaacggaacctgaaccgtttatgaataaccgccgcatggagtgcggacgcggtaaaggtctgggtggatcgtcgctgatcaacggcatgtgctacatccgtggcaatgcgctggatctcgataactgggcgcaagaacccggtctggagaactggagctacctcgactgcctgccctactaccgcaaggccgagactcgcgatatgggtgaaaacgactatcacggcggtgatggcccggtgagcgtcactacctccaaacccggcgtcaatccgctgtttgaagcgatgattgaagcgggcgtgcaggcgggctacccgcgcacggacgatctcaacggttatcagcaggaaggttttggtccgatggatcgcaccgtcacgccgcagggccgtcgcgccagcaccgcgcgtggctatctcgatcaggccaaatcgcgtcctaacctgaccattcgtactcacgctatgaccgatcacatcatttttgacggcaaacgcgcggtgggcgtcgaatggctggaaggcgacagcaccatcccaacccgcgcaacggccaacaaagaagtgctgttatgtgcaggcgcgattgcctcaccgcagatcctgcaacgctccggcgtcggcaacgctgaactgctggcggagtttgatattccgctggtgcatgaattacccggcgtcggcgaaaatcttcaggatcatctggagatgtatctgcaatatgagtgcaaagaaccggtttccctctaccctgccctgcagtggtggaaccagccgaaaatcggtgcggagtggctgtttggcggcactggcgttggtgccagcaaccactttgaagcaggtggatttattcgcagccgtgaggaatttgcgtggccgaatattcagtaccatttcctgccagtagcgattaactataacggctcgaatgcagtgaaagagcacggtttccagtgccacgtcggctcaatgcgctcgccaagccgtgggcatgtgcggattaaatcccgcgacccgcaccagcatccggcgattctgtttaactacatgtcgcacgagcaggactggcaggagttccgcgacgcaattcgcatcacccgcgagatcatgcatcaacccgcgctggatcagtatcgtggccgcgaaatcagccccggtgtcgaatgccagacggatgaacagctcgatgagttcgtgcgtaaccacgccgaaaccgccttccatccgtgcggtacctgcaaaatgggttacgacgagatgtccgtggttgacggcgaaggccgcgtacacgggttagaaggcctgcgtgtggtggatgcgtcgattatgccgcagattatcaccgggaatttgaacgccacgacaattatgattggcgagaaaatagcggatatgattcgtggacaggaagcgctgccgaggagcacggcgggatattttgtggcaaatgggatgccggtgagagcgaaaaaatga

SEQ ID NO: 60 - DNA, 1473 bp, Escherichia coli
atgtcccgaatggcagaacagcagctttatatacatggtggttatacctccgccaccagcggtcgcaccttcgagaccattaacccggccaacggtaacgtgctggcgaccgtgcaggccgccgggcgcgaggatgtcgatcgcgccgtgaaaagcgcccagcaggggcaaaaaatctgggcgtcgatgaccgccatggagcgctcgcgtattctgcgtcgggccgttgatattctgcgtgaacgcaatgacgaactcgcaaaactggaaaccctcgacaccggaaaagcatattcggaaacctcaaccgtcgatatcgttaccggtgcggacgtgctggagtactacgccgggctgatcccggcgctggaaggcagccagatcccgttgcgtgaaacgtcctttgtgtatacccgccgcgaaccgctgggcgtagtggcagggattggcgcatggaactacccgatccagattgccctgtggaaatccgccccggcgctggcggcaggcaacgcaatgattttcaaaccgagcgaagttaccccgcttaccgcgttaaagctggctgaaatttacagcgaagcgggcctgccggacggcgtatttaacgtgttgccgggcgtgggcgcggagaccgggcaatatctgaccgagcatccgggcattgccaaagtgtcatttaccggcggtgtcgccagcggcaaaaaagtgatggctaactcggcggcctcttccctgaaagaagtgaccatggaactgggcggtaaatcaccgctgatcgttttcgatgatgcggatctcgatctcgccgccgatatcgccatgatggcaaacttcttcagctccggtcaggtgtgtaccaatggcacccgcgtcttcgttccggcgaaatgcaaagccgcatttgagcagaaaattctggcgcgcgttgagcgcattcgcgcgggcgacgttttcgatccgcaaactaacttcggcccgctggtcagcttcccgcatcgcgataacgtgctgcgctatatcgccaaaggcaaagaggaaggcgcgcgcgtactgtgcggcggcgatgtactgaaaggcgatggcttcgataacggcgcatgggttgcaccgacagtgttcaccgattgcagcgacgatatgaccatcgtgcgtgaagagatcttcgggccagtgatgtccattctgacctacgagtcggaagacgaagtcattcgccgcgctaacgataccgactacggcctggcggcgggcatcgtgacagcggacctgaaccgcgcgcatcgcgtcattcatcagctggaagcgggtatttgctggatcaacacctggggcgaatccccggcagagatgcccgttggcggctacaaacactccggcattggtcgcgagaacggcgtgatgacgctccagagttacacccaggtgaagtccatccaggttgagatggctaaattccagtccatattctaa

SEQ ID NO: 61 - DNA, 2034 bp, Escherichia coli
atgacagacctttcacacagcagggaaaaggacaaaatcaatccggtggtgttttacacctccgccggactgattttgttgttttccctgacaacgatcctgtttcgcgacttctcggccctgtggattggccgcacgctggactgggtttctaaaaccttcggttggtactatctgctggcggcaacgctctatattgtctttgtggtctgtatcgcttgttcgcgttttggttcggtgaagctcgggccagaacaatccaaaccggaattcagcctgctgagttgggcggcgatgctgtttgctgccgggatcggtatcgacctgatgttcttctccgtagccgaaccggtaacgcagtatatgcagccgccggaaggcgcgggacagacgattgaggccgcgcgtcaggcgatggtctggacgctgtttcactacggcttaaccggctggtcgatgtatgcgctgatgggcatggcgctcggatactttagctatcgttataatttgccgctcaccatccgctcggcgctgtacccgatcttcggtaaacggattaacgggccgataggtcactcagtggatattgcagcggtgatcggcactatcttcggtattgccactacgctcggtatcggtgtggtgcagcttaactatggcttgagcgtactgtttgatattcccgattcgatggcggcgaaagcggcactgatcgccttgtcggtgataatcgccacgatctctgtcacctccggtgtcgataagggcattcgcgtgttatcggagcttaatgtcgcgctggcgctgggattgatcctgttcgtattgtttatgggcgacacttcgttcctgcttaatgcactggtgctgaatgttggcgactatgtgaatcgctttatgggcatgacgctcaacagttttgccttcgaccgtccggttgagtggatgaataactggacgctcttcttctgggcatggtgggtggcatggtcgccgtttgtcggcttgttcctggcgcgtatctcgcgtgggcgtaccattcgccagttcgtgctgggcacgttgattattccgtttaccttcacgctgttatggctctcggtgttcggcaatagcgcgctgtatgaaatcatccacggcggcgcggcatttgccgaggaagcgatggtccatccggagcgcggcttctacagcctgctggcgcagtatccggcgtttacctttagcgcctccgtcgccaccattactggcctgctgttttatgtgacctcggcggactccggggcgctggtgctggggaatttcacctcgcagcttaaagatatcaacagcgacgcccccggctggctgcgcgtcttctggtcggtggcgattggcctgctgacgctcggcatgctgatgactaacgggatatccgcgctgcaaaacaccacggtgattatggggctgccgttcagctttgtgatcttcttcgtgatggcggggttgtataaatctctgaaggtagaagattaccgccgtgaaagtgccaaccgcgataccgcaccgcgaccgctggggcttcaggatcgcctgagctggaaaaaacgtctctcgcgcctgatgaattatccgggcacgcgttacactaaacagatgatggagacggtctgttacccggcaatggaagaagtggcgcaggagttgcggttgcgcggcgcgtacgtggagctaaaaagcctgccaccggaagagggacagcagttgggtcatctggatttgttggtgcatatgggcgaagagcaaaactttgtctatcagatttggccgcagcaatattcggtgccgggctttacctaccgcgcacgcagcggtaaatcgacctactaccggctggaaaccttcctgttagaaggcagccagggcaacgacctgatggactacagcaaagagcaggtgatcaccgatattcttgaccagtacgagcggcaccttaactttattcatctccatcgtgaagcgccgggccatagcgtgatgttcccggacgcgtga

SEQ ID NO: 62 - DNA, 432 bp, Escherichia coli
atgacaatccataagaaaggtcaggcacactgggaaggcgatatcaaacgcgggaagggaacagtatccaccgagagtggcgtgctgaaccaacagccgtatggatttaacacgcgttttgaaggcgaaaaaggaaccaaccctgaagaactgattggcgcagcgcatgccgcatgtttctcaatggcgctttcattaatgctgggggaagcgggattcacgccaacatcgattgataccaccgccgatgtgtcgctggataaagtggatgccggttttgcgattacgaaaatcgcactgaagagtgaagttgcggtgccgggtattgatgcctctacctttgacggcataatccagaaagcaaaagcaggatgcccggtctctcaggtactgaaagcggaaattacgctggattaccagttgaaatcgtaa

SEQ ID NO: 63 - DNA, 918 bp, Escherichia coli
atgaatattcgtgatcttgagtacctggtggcattggctgaacaccgccattttcggcgtgcggcagattcctgccacgttagccagccgacgcttagcgggcaaattcgtaagctggaagatgagctgggcgtgatgttgctggagcggaccagccgtaaagtgttgttcacccaggcgggaatgctgctggtggatcaggcgcgtaccgtgctgcgtgaggtgaaagtccttaaagagatggcaagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgggtttctgttttgaagccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 64 - DNA, 324 bp, Escherichia coli
atgtcccatcagaaaattattcaggatcttatcgcatggattgacgagcatattgaccagccgcttaacattgatgtagtcgcaaaaaaatcaggctattcaaagtggtacttgcaacgaatgttccgcacggtgacgcatcagacgcttggcgattacattcgccaacgccgcctgttactggccgccgttgagttgcgcaccaccgagcgtccgatttttgatatcgcaatggacctgggttatgtctcgcagcagaccttctcccgcgttttccgtcggcagtttgatcgcactcccagcgattatcgccaccgcctgtaa

SEQ ID NO: 65 - DNA, 384 bp, Escherichia coli
atgtccagacgcaatactgacgctattaccattcatagcattttggactggatcgaggacaacctggaatcgccactgtcactggagaaagtgtcagagcgttcgggttactccaaatggcacctgcaacggatgtttaaaaaagaaaccggtcattcattaggccaatacatccgcagccgtaagatgacggaaatcgcgcaaaagctgaaggaaagtaacgagccgatactctatctggcagaacgatatggcttcgagtcgcaacaaactctgacccgaaccttcaaaaattactttgatgttccgccgcataaataccggatgaccaatatgcagggcgaatcgcgctttttacatccattaaatcattacaacagctag

SEQ ID NO: 66 - DNA, 855 bp, Escherichia coli
atgactgacaaaatgcaaagtttagctttagccccagttggcaacctggattcctacatccgggcagctaacgcgtggccgatgttgtcggctgacgaggagcgggcgctggctgaaaagctgcattaccatggcgatctggaagcagctaaaacgctgatcctgtctcacctgcggtttgttgttcatattgctcgtaattatgcgggctatggcctgccacaggcggatttgattcaggaaggtaacatcggcctgatgaaagcagtgcgccgtttcaacccggaagtgggtgtgcgcctggtctccttcgccgttcactggatcaaagcagagatccacgaatacgttctgcgtaactggcgtatcgtcaaagttgcgaccaccaaagcgcagcgcaaactgttcttcaacctgcgtaaaaccaagcagcgtctgggctggtttaaccaggatgaagtcgaaatggtggcccgtgaactgggcgtaaccagcaaagacgtacgtgagatggaatcacgtatggcggcacaggacatgacctttgacctgtcttccgacgacgattccgacagccagccgatggctccggtgctctatctgcaggataaatcatctaactttgccgacggcattgaagatgataactgggaagagcaggcggcaaaccgtctgaccgacgcgatgcagggtctggacgaacgcagccaggacatcatccgtgcgcgctggctggacgaagacaacaagtccacgttgcaggaactggctgaccgttacggcgtttccgctgagcgtgtacgccagctggaaaagaacgcgatgaaaaaattgcgtgctgccattgaagcgtaa

SEQ ID NO: 67 - DNA, 1497 bp, Artemisia annua
catatgaagtctattctgaaagcaatggctctgtctctgaccactagcatcgccctggcgactatcctgctgtttgtgtacaaattcgcgacccgttctaaaagcactaagaaatctctgccggaaccgtggcgtctgccaatcatcggtcacatgcaccacctgatcggcaccaccccgcaccgtggcgtacgcgacctggcgcgtaagtacggctctctgatgcatctgcagctgggcgaggtacctactatcgtcgtttcctccccgaagtgggccaaagaaatcctgactacctatgacatcactttcgccaaccgcccggaaacgctgaccggcgaaattgtcctgtaccataacacggatgtggttctggccccgtacggtgagtactggcgccagctgcgcaaaatttgtactctggaactgctgagcgttaaaaaggttaaatccttccagagcctgcgtgaagaggaatgctggaacctggtgcaggagattaaagcgtctggcagcggtcgtccagttaacctgtctgagaatgtttttaaactgatcgctactatcctgtctcgcgcggcattcggtaaaggtatcaaagatcagaaagaactgaccgaaatcgttaaggaaatcctgcgccagactggtggcttcgacgttgcggacatcttcccgtccaaaaagttcctgcaccatctgtctggcaaacgcgctcgtctgacctccctgcgtaagaaaattgataacctgattgacaacctggtcgctgagcacactgtgaacacctcttctaaaaccaacgaaaccctgctggacgtactgctgcgcctgaaggactctgccgaatttccactgactagcgacaatatcaaagcaatcatcctggacatgttcggcgccggtaccgatacgtcctcttccacgattgagtgggctatttccgaactgatcaaatgcccgaaggcgatggaaaaagtgcaggcggaactgcgtaaagcgctgaacggtaaagagaaaattcatgaagaggacatccaggaactgtcctacctgaatatggtaatcaaagaaactctgcgtctgcatccgccgctgccactggttctgccgcgtgaatgccgtcagccggttaacctggccggctacaacattccgaacaaaacgaagctgatcgtcaacgttttcgcgatcaaccgcgatcctgaatactggaaagacgcggaagcgttcattccggaacgctttgagaactcctctgccaccgttatgggcgctgaatacgagtacctgccgttcggtgcgggtcgccgtatgtgcccgggtgctgcactgggcctggcgaacgttcaactgccactggcgaacatcctgtaccacttcaactggaaactgcctaacggcgtatcttatgatcaaatcgacatgaccgaaagctccggcgcgaccatgcagcgtaaaaccgaactgctgctggttccgtccttttaacctagg

SEQ ID NO: 68 - DNA, 1497 bp, Artificial Sequence; identical to SEQ ID NO: 67
  synthetic nucleic acid

SEQ ID NO: 69 - DNA, 3018 bp, Artificial Sequence
  synthetic nucleic acid
catatgaccgtacacgacatcatcgcaacgtacttcactaaatggtacgtaattgtgccgctggcactgattgcgtatcgcgtgctggattatttctacgcgacccgttctaaaagcactaagaaatctctgccggaaccgtggcgtctgccaatcatcggtcacatgcaccacctgatcggcaccaccccgcaccgtggcgtacgcgacctggcgcgtaagtacggctctctgatgcatctgcagctgggcgaggtacctactatcgtcgtttcctccccgaagtgggccaaagaaatcctgactacctatgacatcactttcgccaaccgcccggaaacgctgaccggcgaaattgtcctgtaccataacacggatgtggttctggccccgtacggtgagtactggcgccagctgcgcaaaatttgtactctggaactgctgagcgttaaaaaggttaaatccttccagagcctgcgtgaagaggaatgctggaacctggtgcaggagattaaagcgtctggcagcggtcgtccagttaacctgtctgagaatgtttttaaactgatcgctactatcctgtctcgcgcggcattcggtaaaggtatcaaagatcagaaagaactgaccgaaatcgttaaggaaatcctgcgccagactggtggcttcgacgttgcggacatcttcccgtccaaaaagttcctgcaccatctgtctggcaaacgcgctcgtctgacctccctgcgtaagaaaattgataacctgattgacaacctggtcgctgagcacactgtgaacacctcttctaaaaccaacgaaaccctgctggacgtactgctgcgcctgaaggactctgccgaatttccactgactagcgacaatatcaaagcaatcatcctggacatgttcggcgccggtaccgatacgtcctcttccacgattgagtgggctatttccgaactgatcaaatgcccgaaggcgatggaaaaagtgcaggcggaactgcgtaaagcgctgaacggtaaagagaaaattcatgaagaggacatccaggaactgtcctacctgaatatggtaatcaaagaaactctgcgtctgcatccgccgctgccactggttctgccgcgtgaatgccgtcagccggttaacctggccggctacaacattccgaacaaaacgaagctgatcgtcaacgttttcgcgatcaaccgcgatcctgaatactggaaagacgcggaagcgttcattccggaacgctttgagaactcctctgccaccgttatgggcgctgaatacgagtacctgccgttcggtgcgggtcgccgtatgtgcccgggtgctgcactgggcctggcgaacgttcaactgccactggcgaacatcctgtaccacttcaactggaaactgcctaacggcgtatcttatgatcaaatcgacatgaccgaaagctccggcgcgaccatgcagcgtaaaaccgaactgctgctggttccgtccttttaacctaggcatatgaccgtacacgacatcatcgcaacgtacttcactaaatggtacgtaattgtgccgctggcactgattgcgtatcgcgtgctggattatttctacgcgacccgttctaaaagcactaagaaatctctgccggaaccgtggcgtctgccaatcatcggtcacatgcaccacctgatcggcaccaccccgcaccgtggcgtacgcgacctggcgcgtaagtacggctctctgatgcatctgcagctgggcgaggtacctactatcgtcgtttcctccccgaagtgggccaaagaaatcctgactacctatgacatcactttcgccaaccgcccggaaacgctgaccggcgaaattgtcctgtaccataacacggatgtggttctggccccgtacggtgagtactggcgccagctgcgcaaaatttgtactctggaactgctgagcgttaaaaaggttaaatccttccagagcctgcgtgaagaggaatgctggaacctggtgcaggagattaaagcgtctggcagcggtcgtccagttaacctgtctgagaatgtttttaaactgatcgctactatcctgtctcgcgcggcattcggtaaaggtatcaaagatcagaaagaactgaccgaaatcgttaaggaaatcctgcgccagactggtggcttcgacgttgcggacatcttcccgtccaaaaagttcctgcaccatctgtctggcaaacgcgctcgtctgacctccctgcgtaagaaaattgataacctgattgacaacctggtcgctgagcacactgtgaacacctcttctaaaaccaacgaaaccctgctggacgtactgctgcgcctgaaggactctgccgaatttccactgactagcgacaatatcaaagcaatcatcctggacatgttcggcgccggtaccgatacgtcctcttccacgattgagtgggctatttccgaactgatcaaatgcccgaaggcgatggaaaaagtgcaggcggaactgcgtaaagcgctgaacggtaaagagaaaattcatgaagaggacatccaggaactgtcctacctgaatatggtaatcaaagaaactctgcgtctgcatccgccgctgccactggttctgccgcgtgaatgccgtcagccggttaacctggccggctacaacattccgaacaaaacgaagctgatcgtcaacgttttcgcgatcaaccgcgatcctgaatactggaaagacgcggaagcgttcattccggaacgctttgagaactcctctgccaccgttatgggcgctgaatacgagtacctgccgttcggtgcgggtcgccgtatgtgcccgggtgctgcactgggcctggcgaacgttcaactgccactggcgaacatcctgtaccacttcaactggaaactgcctaacggcgtatcttatgatcaaatcgacatgaccgaaagctccggcgcgaccatgcagcgtaaaaccgaactgctgctggttccgtccttttaacctagg

SEQ ID NO: 70 - DNA, 16191 bp, Artificial Sequence
  synthetic nucleic acid
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttccgatggcgcgccgagaggctttacactttatgcttccggctcgtataatgtgtggaattgtgagcggataacaattgaattcaaaggaggccatcctggccatgaagaactgtgtgattgtttctgcggtccgcacggcgatcggcagctttaacggctctttagcgagcacctctgcaatcgatctgggtgcgacggtcattaaggccgccattgaacgcgccaaaatcgacagccagcacgttgatgaggtgatcatgggcaatgtgttacaagccggcctgggtcaaaacccagcgcgtcaagcactgttaaaatctggtctggccgagaccgtgtgtggcttcaccgtcaataaggtttgcggctctggcctgaagagcgtggccctggcagcacaagcgattcaagccggtcaggcacaaagcatcgttgcgggtggcatggagaacatgtctctggcgccgtacttattagatgccaaagcccgcagcggttatcgcctgggcgatggtcaggtgtacgacgtcatcttacgcgatggcttaatgtgcgcgacccacggttaccacatgggtattacggccgaaaacgtggcgaaagaatacggcattacgcgcgagatgcaggatgaattagcactgcactctcagcgcaaagcagcagccgcgatcgagtctggtgcgtttacggcggaaatcgtgccagttaacgtggtcacgcgcaagaagacgttcgttttcagccaggacgagttcccgaaggcaaacagcaccgcggaggccttaggtgccttacgcccagcctttgacaaagcgggcacggtcaccgccggtaatgcgagcggcatcaatgatggtgcagcggcactggtcatcatggaagagagcgccgcattagcagcgggtctgaccccattagcgcgcattaaatcttatgccagcggcggcgtcccaccagccctgatgggcatgggtccggtcccagccacgcaaaaagccctgcaattagcgggcctgcaactggccgacattgatctgatcgaggcgaacgaggcgtttgcagcgcagttcctggcggtgggtaagaatctgggcttcgacagcgagaaagtcaatgtgaacggtggcgcgattgcgttaggccatccgattggtgcaagcggcgcacgcatcttagtgacgttactgcacgccatgcaggcacgcgacaagaccttaggcctggcgaccttatgtattggtggcggtcaaggtatcgccatggtgatcgaacgcctgaactgaagatctaggaggaaagcaaaatgaaactgagcaccaagctgtgctggtgtggcatcaagggtcgcctgcgcccacaaaagcagcaacagctgcacaacacgaacctgcaaatgaccgagctgaaaaagcagaagacggccgagcaaaagacccgcccgcagaacgttggcatcaagggcatccagatttatatcccgacgcagtgtgtcaaccaatctgagctggagaaattcgatggcgtcagccagggtaagtacaccatcggcctgggccagaccaacatgagcttcgtgaacgaccgtgaggacatctattctatgagcctgacggtgctgtctaagctgatcaagagctacaacatcgacacgaataagatcggtcgtctggaggtgggtacggagacgctgattgacaagagcaaaagcgtgaagtctgtcttaatgcagctgttcggcgagaacacggatgtcgagggtatcgacaccctgaacgcgtgttacggcggcaccaacgcactgttcaatagcctgaactggattgagagcaacgcctgggatggccgcgatgcgatcgtcgtgtgcggcgatatcgccatctatgacaagggtgcggcacgtccgaccggcggtgcaggcaccgttgcgatgtggattggcccggacgcaccaattgtcttcgattctgtccgcgcgtcttacatggagcacgcctacgacttttacaagccggacttcacgagcgaatacccgtacgtggacggccacttctctctgacctgctatgtgaaggcgctggaccaggtttataagtcttatagcaaaaaggcgatttctaagggcctggtcagcgacccggcaggcagcgacgccctgaacgtgctgaagtatttcgactacaacgtgttccatgtcccgacctgcaaattagtgaccaaatcttatggccgcctgttatataatgatttccgtgccaacccgcagctgttcccggaggttgacgccgagctggcgacgcgtgattacgacgagagcctgaccgacaagaacatcgagaagaccttcgtcaacgtcgcgaagccgttccacaaagagcgtgtggcccaaagcctgatcgtcccgaccaacacgggcaacatgtataccgcgtctgtctacgcggcattcgcgagcctgctgaattacgtcggttctgacgacctgcagggcaagcgcgttggcctgttcagctacggtagcggcttagcggccagcctgtatagctgcaaaattgtcggcgacgtccagcacatcatcaaggagctggacatcaccaacaagctggcgaagcgcatcaccgagacgccgaaagattacgaggcagcgatcgagttacgcgagaatgcgcatctgaagaagaacttcaagccgcaaggtagcatcgagcacctgcagagcggcgtctactacctgacgaacattgacgacaagttccgccgttcttatgacgtcaaaaagtaactagtaggaggaaaacatcatggtgctgacgaacaaaaccgtcattagcggcagcaaggtgaagtctctgagcagcgcccaaagctctagcagcggcccgtctagcagcagcgaggaggacgacagccgtgacattgagtctctggacaagaagatccgcccgctggaggagttagaggccctgctgagcagcggcaacaccaagcagctgaagaacaaggaagttgcagcgctggtgatccacggtaagctgccactgtatgcgctggaaaagaaactgggcgatacgacgcgtgcggtcgcggtgcgtcgcaaagccttaagcatcttagcggaggccccggtgttagccagcgaccgcctgccgtacaagaactacgactacgaccgcgtgtttggcgcgtgctgcgagaatgtcattggctacatgccgttaccggttggtgtgatcggcccgctggtcattgatggcacgagctatcacattccaatggcgaccacggaaggttgcttagtcgccagcgccatgcgtggctgtaaggcgattaacgccggcggtggcgcgacgaccgtgttaaccaaggatggtatgacgcgcggtccggtcgtccgcttcccaacgctgaagcgcagcggcgcgtgtaagatttggctggattctgaggagggccaaaacgcgatcaagaaagccttcaactctacgagccgtttcgcgcgtttacagcatatccagacctgcctggccggcgacctgctgttcatgcgcttccgcaccaccacgggcgatgcgatgggcatgaacatgatcagcaagggcgtcgaatatagcctgaaacaaatggtggaagaatatggctgggaggacatggaggttgtctctgtgagcggcaactattgcaccgacaagaagccggcagccattaactggattgagggtcgcggcaaaagcgtcgtggcagaagcgaccatcccaggcgacgtggtccgtaaggttctgaagagcgacgtcagcgccctggttgagttaaatatcgcgaaaaacctggtcggcagcgcgatggcgggcagcgtgggtggctttaacgcacatgcagcgaatctggttacggcggttttcttagccttaggtcaggacccagcccaaaatgtcgagagcagcaactgcattaccttaatgaaagaggttgacggtgacctgcgcatcagcgtttctatgccgtctatcgaggtcggcacgatcggcggcggcaccgttttagaaccgcaaggtgcgatgctggatctgctgggcgtgcgcggcccacatgcaacggccccaggcaccaatgcccgccaactggcccgtatcgtggcctgcgcggttctggcgggtgagctgagcctgtgcgccgcattagccgcgggccatttagttcaatctcacatgacccacaaccgcaagccggcagaaccaaccaagccaaataacctggacgcaaccgacattaaccgtctgaaggatggcagcgtcacgtgcattaaaagctgagcatgctactaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgcgatcgccgagaggctttacactttatgcttccggctcgtataatgtgtggaattgtgagcggataacaattgaattcaaaggaggctcgagatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataggaggcagatcaaatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataggaggtaatactcatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataactgcagcccgggaggaggattactatatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataacccgggggatccactagttctagagcggccgccaccgcggaggaggaatgagtaatggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataagagctccaattcgccctatagtgagacgcgtgctagaggcatcaaataaaacgaaaggctcagtcgaaagactgggcctttcgttttatctgttgtttgtcggtgaacgctctcctgagttaattaatcagatggacatcgggtaaaccagcagggatttgatcaggtgtttgtattcgtcgcccatgcgagtgaagttatctttaccagcgtactgtacttccaggaactggcacaggtagattactgccatcagcagcgggcgcgggatgtttttagtagtcaggtattcacggttgatgtctttccatacgtcttcaacttctttatagatcagagtctgtgcgtactcctcgttaacgttatattccttcatgtaggattccagagaggaggaagagtgtttacgttcctgctctgctttgtgggtcatcaggtcgttcagacgacgacccagaataccggagtaacggaacagcggcggtgcagaaacagcccattcaacagattccttggtaaagatgtcggacatacccagatagcaagtggtggtcagcaggtttgcaccgccggtgatgataacaaccgggtcatgttcttcggtagtcgggatatggccttcgttagcccatttagcttcaaccatcaggttacgtacgaattctttaacaaactctttaccgcagttgaacaggtcggtacggccttcttttgccaggaattcctccatttcggtgtaggtatccatgaacagtttgtagatcggtttcatgtactccggcagagtgtccaggcaagtgatagaccagcgttctacagcttcagtaaagatcttcagttcttcgtaggtgccgtaagcatcgtaagtgtcatcgatcagggtgataacagctacagctttagtgaagaacacacgtgcacgggagtactgtggttcataaccagaacccagaccccagaagtaacattcaacgatacggtcacgcaggcacggcgcgtttttcttgatgtcaaatgccttccaccacttacaaacgtgagacagttcttctttgtgcagagactgcagcaggttgaattccagcttagccagtttcagcagggtcttgttgtgagagtcctgctgctggtaaaacggaatgtactgtgctgcttcgatacgcggcagacgtttccacagcggctgtttcagagcacgctggatttcggtgaacagagccgggttagtagagaaagcgtctttagtcataatggacagacgagaacgggtgaaacccagcgcgtcctccaggatgatttcacccggtacacgcatggaggtcgcttcgtacagttccagcaggccttcaacgtcgttagccagagactgtttgaaagcaccgttcttgtccttgtagttgttaaaaacgtcacaggtaacgtagtagccctgtttacgcatcagacgaaaccacagagaagaacggtcgccgttccagttgtcgccgtaggtttcgtagatgcactgcagtgcgtggtcgatttcgcgttcgaagtggtacgggatacccagacgctggatctcgtcgatcagtttcagcaggttagcgtgtttcatcgggatgtccagagcttctttcagcagctgacgaacttctttcttcaggtcgtttacgatctgttcaacaccctgctcaacctgcttttcgtagatcaggaactggtcaccccagatagacggcgggaagttagcgatcgggcggatcggtttctcttcggtcagggccatggtctgtttcctgtgtgaaattgttatccgctcacaattccacacattatacgagccggatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcaccgtgcagtcgatgataagctgtcaaaccagatcaattcgcgctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgggccggcctacgcgtttaaacttccggttaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 71 - DNA, 2542 bp, Escherichia coli
aaggagatatacatttgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttacgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctgacccgggaaggagatatacatatgatcaagctcggcatcgtgatggaccccatcgcaaacatcaacatcaagaaagattccagttttgctatgttgctggaagcacagcgtcgtggttacgaacttcactatatggagatgggcgatctgtatctgatcaatggtgaagcccgcgcccatacccgcacgctgaacgtgaagcagaactacgaagagtggttttcgttcgtcggtgaacaggatctgccgctggccgatctcgatgtgatcctgatgcgtaaagacccgccgtttgataccgagtttatctacgcgacctatattctggaacgtgccgaagagaaagggacgctgatcgttaacaagccgcagagcctgcgcgactgtaacgagaaactgtttaccgcctggttctctgacttaacgccagaaacgctggttacgcgcaataaagcgcagctaaaagcgttctgggagaaacacagcgacatcattcttaagccgctggacggtatgggcggcgcgtcgattttccgcgtgaaagaaggcgatccaaacctcggcgtgattgccgaaaccctgactgagcatggcactcgctactgcatggcgcaaaattacctgccagccattaaagatggcgacaaacgcgtgctggtggtggatggcgagccggtaccgtactgcctggcgcgtattccgcaggggggcgaaacccgtggcaatctggctgccggtggtcgcggtgaacctcgtccgctgacggaaagtgactggaaaatcgcccgtcagatcgggccgacgctgaaagaaaaagggctgatttttgttggtctggatatcatcggcgaccgtctgactgaaattaacgtcaccagcccaacctgtattcgtgagattgaagcagagtttccggtgtcgatcaccggaatgttaatggatgccatcgaagcacgtttacagcagcagtaa

SEQ ID NO: 72 - DNA, 14 bp, Artificial Sequence
  synthetic ribosome binding site
aaggagatatacat

SEQ ID NO: 73 - DNA, 5527 bp, Escherichia coli
aaggagatatacatatggacatgcattcaggaacctttaacccacaagatttcgcctggcaaggcttaacgctgacacccgcagcggcgatacacatccgtgagctggtggcaaagcagccgggtatggtcggcgtgcgcttaggcgtgaagcaaacgggctgcgcgggctttggctatgtgctcgacagtgttagcgagccggacaaagacgatctgctgtttgaacacgacggcgcgaagctgtttgtcccgctgcaagcgatgccgtttattgatggcacggaagtcgatttcgttcgtgaaggacttaatcagatattcaaatttcacaaccctaaagcccagaatgaatgtggctgtggcgaaagctttggggtataggcggtactatgtctcgtaatactgaagcaactgacgatgtcaaaacctggaccggcggcccgctgaattataaagaaggattcttcacccagttagccaccgatgagctggcaaaggggataaacgaagaggtggtgcgcgcaatttcggcgaagcgtaatgagccggagtggatgctggagtttcgtctaaacgcctatcgcgcatggctggagatggaagaaccgcactggttgaaagcgcactacgacaagctgaattatcaggattacagctactactcagcaccatcgtgcggtaattgtgacgacacttgcgcgtctgaacctggcgcggtgcagcaaactggcgcgaacgcctttttaagtaaagaggtggaggcggcgtttgagcagttgggcgttcccgtgcgggaaggcaaagaggtggcggtggatgccattttcgactcagtttcggttgccactacttatcgcgaaaaactggcggagcagggaattattttctgttcctttggtgaggcgatccacgatcacccggaactggtgcgtaaatatctcggcaccgtggtgccggggaatgacaacttctttgccgcgcttaatgcggcggtagcctctgatggtacgtttatttatgtgcctaaaggcgtgcgctgcccgatggaactttccacctattttcgcattaacgcagaaaaaaccgggcagtttgagcgcaccattctggtggccgacgaagacagctacgtcagctacattgaaggctgttccgctccggtgcgtgacagctatcagttacacgcggcagtggtggaagtcatcatccataaaaacgccgaggtgaaatattccacggtacaaaactggtttcctggcgataacaacaccggcggtattctcaacttcgtcaccaagcgtgctttgtgcgaaggcgaaaacagcaaaatgtcatggacgcaatcagaaaccgggtcagcgattacgtggaaatatcccagctgcattttgcgcggcgataactccattggtgagttttactcagtggcgctgaccagcggtcatcagcaagcggataccggcaccaagatgatccacatcggtaaaaacaccaaatcgaccattatctcgaaagggatctctgccggacatagtcagaacagttatcgcggcttagtgaaaatcatgccgacggcaaccaatgcgcgcaatttcactcagtgcgactcaatgctgattggcgctaattgtggggcgcataccttcccgtatgttgagtgtcgtaacaatagtgcgcaactggaacacgaggcaacgacatcacgtattggtgaagatcaactgttttactgcctgcaacgcgggatcagcgaagaagacgccatctcgatgattgttaacggtttctgcaaagacgtgttctcggagctgccgttggaatttgccgttgaagcacaaaaactcctcgccatcagtcttgaacacagcgtcggataaggaataaacatgttaagtattaaagatttacacgtcagcgtggaagataaagctatcctgcgcggattaagcctcgacgttcatcccggcgaagttcacgccattatggggccaaacggttcgggcaaaagtaccttatcggcaacgcttgccgggcgagaagattatgaagtgacgggcggcacggttgagttcaaaggcaaagatttgcttgcgctgtcgccggaagatcgcgcgggcgaaggcatctttatggccttccagtatccggtggagattccaggtgtcagtaaccagtttttcctgcaaacggcacttaatgcggtgcgcagctatcgcggccaggaaacgctcgaccgctttgattttcaggatttgatggaagagaaaatcgctctcctgaagatgccggaagatttattaacccgttcggtaaacgttggtttttccggcggcgagaaaaagcgcaacgatattttgcaaatggcggtgctggaaccggagttatgcattcttgatgagtcggactccgggctggatattgacgcattaaaagtggtcgccgatggcgtgaactcgctgcgtgatggcaagcgctcattcatcattgttacgcactaccaacgcattctcgactacatcaagcctgattacgttcatgtgctatatcagggacgaattgtgaaatccggcgatttcacgttggtcaaacaactggaggagcagggttatggctggcttaccgaacagcagtaacgcgctgcaacagtggcatcacttgtttgaagctgaagggacaaaacgctccccgcaagcacagcagcatttacaacaattgctgcgtaccggactgccgacacgtaaacatgaaaactggaaatatacgccgctggaagggctgatcaatagccagtttgtcagcattgcgggagagatatccccacagcagcgtgatgccttagcgttaacgttagactccgtgcggctggtgtttgtcgatgggcgttacgtgcccgcactgagcgatgcaactgaaggcagcggatatgaagtgagcattaacgacgaccgtcagggtttacccgacgctattcaggcggaagtgtttctgcatttgacggaaagcctggcacaaagcgtgacgcatatcgccgtgaagcgcggtcaacggccggcaaagccattgctgttaatgcatatcacccagggcgtggcaggtgaagaggtgaacactgcccattaccgacatcatctggatctggcggaaggtgccgaagcaacggtgatcgaacattttgtcagcctgaatgatgctcgtcattttaccggggcacggttcactatcaacgtcgcagcgaatgcccacttgcagcatatcaagctggcgtttgaaaacccgctcagtcaccactttgctcataacgatttgttgctggctgaggatgccaccgcatttagccacagtttcctgctgggtggcgcagtgttacgacacaacaccagtacgcaactcaatggcgaaaacagcacgctgcggatcaatagcctggcgatgccggtgaaaaacgaggtgtgtgatacccgtacctggctggaacacaataaaggtttttgtaacagccgacagttgcacaaaactatcgtcagcgacaaaggccgcgcggtatttaacggtttgatcaacgtcgcgcagcacgccatcaaaacggatggtcagatgaccaacaacaatctgctgatgggcaaactggcggaagtggatacgaaaccgcagctggaaatctatgcagatgatgtgaaatgcagccacggcgcgacggtggggcgtattgatgatgaacagatattctatctgcgctcgcgcgggatcaatcagcaggatgcccagcagatgatcatttacgccttcgctgccgaactgacggaagcactgcgtgatgaggggcttaaacagcaggtgctggcccgaatcggtcaacggctgccaggaggtgcaagatgattttttccgtcgacaaagtgcgggccgactttccggtgctttcgcgtgaggtaaacggtttgccgctggcttatctcgacagcgccgccagtgcgcagaaaccgagccaggtgattgacgccgaggccgagttttatcgtcatggctacgcggcggtgcatcgtggtattcataccttaagcgcccaggcgaccgagaaaatggagaacgtgcgcaagcgggcatcgctgtttattaatgcccgttcggcggaagagctggtgttcgtccgcggcacgacggaagggatcaatctggtcgccaatagctggggcaacagcaacgtgcgggcgggcgataacatcatcatcagtcagatggagcaccacgctaacattgttccctggcagatgctttgcgcacgcgttggcgcagagctgcgtgtgatcccgctcaatcccgatggtacgttgcaactggagacgctgcctacgctgtttgatgagaaaactcgcctgctggcaattactcatgtctccaacgtgcttggcacagaaaatccactggcggaaatgatcacgcttgcgcaccagcatggcgcaaaagtgctggtggatggcgctcaggcggtgatgcatcatccggtggatgttcaggcgctggattgcgacttttacgtgttctccgggcataaactgtatggccccaccggaattggcattctttatgtgaaagaagccttgttgcaggagatgccgccgtgggaagggggcggttctatgatcgccaccgtcagcctgagtgaaggcactacctggaccaaagcaccatggcggtttgaagccggtacacccaataccgggggcatcattggtcttggcgcggcgctggagtatgtttcggcgctggggcttaataacatagccgagtatgaacagaatctgatgcattatgcgctatcacagctggaatctgtaccggatctcactctctatggcccacaaaacaggcttggcgttattgcttttaatctcggtaaacaccacgcctatgatgttggcagttttctcgataattacggcattgctgtgcgtaccggacatcactgcgcaatgccattgatggcctattacaacgtccctgcgatgtgtcgggcgtcgctggccatgtataacacccatgaagaagtggatcgtctggtgaccggcctgcaacgtattcaccgtttgctgggataacagggaggcactatggctttattgccggataaagaaaagttgctgcgtaattttttacgctgcgccaactgggaagagaaatatctctacattattgagctgggccagcgtctgccagaattacgcgacgaagacagaagtccacaaaatagcattcagggctgtcagagtcaggtgtggattgtcatgcgccagaatgcccagggaattattgaattacagggcgacagcgatgcggcgattgtgaaagggcttattgcggtcgtctttattctctacgatcagatgacgccgcaggatattgtcaatttcgatgtgcgtccgtggtttgaaaaaatggcgctcacccaacatctcaccccatctcgttcacaaggtctggaagcgatgattcgcgcaattcgcgccaaagccgctgcacttagctaa

SEQ ID NO: 74 - protein, 19 aa, Artificial Sequence
  synthetic transmembrane domain
MWLLLIAVFLLTLAYLFWP

SEQ ID NO: 75 - protein, 20 aa, Artificial Sequence
  synthetic transmembrane domain
MALLLAVFLGLSCLLLLSLW

SEQ ID NO: 76 - protein, 18 aa, Artificial Sequence
  synthetic transmembrane domain
MAILAAIFALVVATATRV

SEQ ID NO: 77 - protein, 24 aa, Artificial Sequence
  synthetic transmembrane domain
MDASLLLSVALAVVLIPLSLALLN

SEQ ID NO: 78 - protein, 27 aa, Artificial Sequence
  synthetic transmembrane domain
MIEQLLEYWYVVVPVLYIIKQLLAYTK

SEQ ID NO: 79 - protein, 21 aa, Artificial Sequence
  synthetic secretion signal
MKKTAIAIAVALAGFATVAQA

SEQ ID NO: 80 - protein, 21 aa, Artificial Sequence
  synthetic secretion signal
MKKTAIAIVVALAGFATVAQA

SEQ ID NO: 81 - protein, 21 aa, Artificial Sequence
  synthetic secretion signal
MKKTALALAVALAGFATVAQA

SEQ ID NO: 82 - protein, 26 aa, Artificial Sequence
  synthetic secretion signal
MKIKTGARILALSALTTMMFSASALA

SEQ ID NO: 83 - protein, 25 aa, Artificial Sequence
  synthetic secretion signal
MNMKKLATLVSAVALSATVSANAMA

SEQ ID NO: 84 - protein, 21 aa, Artificial Sequence
  synthetic secretion signal
MKQSTIALALLPLLFTPVTKA

SEQ ID NO: 85 - protein, 24 aa, Artificial Sequence
  synthetic solubilization domain
EELLKQALQQAQQLLQQAQELAKK

SEQ ID NO: 86 - protein, 32 aa, Artificial Sequence
  synthetic solubilization domain
MTVHDIIATYFTKWYVIVPLALIAYRVLDYFY

SEQ ID NO: 87 - protein, 29 aa, Artificial Sequence
  synthetic solubilization domain
GLFGAIAGFIEGGWTGMIDGWYGYGGGKK

SEQ ID NO: 88 - protein, 9 aa, Artificial Sequence
  synthetic solubilization domain
MAKKTSSKG

(19) United States
(12) Patent Application Publication
(10) Pub. No.: US 2008/0233623 A1
(43) Pub. Date: Sep. 25, 2008

(54) GENETICALLY MODIFIED HOST CELLS FOR INCREASED P450 ACTIVITY LEVELS AND METHODS OF USE THEREOF

(75) Inventors: Michelle Chia-Yu Chang, Berkeley, CA (US); Rachel A. Krupa, San Francisco, CA (US); Jeffrey Lance Kizer, San Francisco, CA (US); John R. Haliburton, San Francisco, CA (US); Mario Ouellet, El Cerrito, CA (US); Jeffrey Alan Dietrich, Berkeley, CA (US); Jay D. Keasling, Berkeley, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Regents of the University of California

(21) Appl. No.: 12/021,974

(22) Filed: Jan. 29, 2008

Related U.S. Application Data
(60) Provisional application No. 60/887,493, filed on Jan. 31, 2007.

(51) Int. Cl. C12P 5/02; C12N 5/00

(52) U.S. Cl. 435/167; 435/325

(57) ABSTRACT

The present invention provides genetically modified host cells that exhibit modified activity levels of one or more gene products such that, when a cytochrome P450 enzyme is produced in the genetically modified host cell, the modified activity levels of the one or more gene products provide for enhanced production and/or activity of the cytochrome P450 enzyme. The present invention provides methods of producing a cytochrome P450 enzyme in a host cell, generally involving culturing a subject genetically modified host cell in a suitable culture medium. The present invention further provides methods of producing a product of a P450-dependent oxidation, generally involving culturing a subject genetically modified host cell in a suitable culture medium.

### CROSS-REFERENCE

This application claims the benefit of U.S. Provisional Patent Application No. 60/887,493, filed Jan. 31, 2007, which application is incorporated herein by reference in its entirety.

### BACKGROUND

Natural products have provided a rich source for discovery of pharmacologically-active small molecules. However, since they are typically produced in small quantities in their native hosts, isolation from biological sources suffers from low yields and high consumption of limited natural resources. Furthermore, the multiple steps required for chemical synthesis of natural products are often difficult to scale for industrial production. An alternative approach to production of natural products or their semisynthetic precursors of transplanting the biosynthetic pathway from the native host into genetically-engineered microorganisms such as Escherichia coli, allowing us to isolate large quantities of complex small molecules using relatively inexpensive fermentation methods.

One of the most important classes of enzymes in the biochemical transformations of many natural product targets is the cytochrome P450 (P450) superfamily, which takes part in a wide spectrum of metabolic reactions. Cytochrome P450 enzymes (P450s) are membrane-bound heme monooxygenases that are ubiquitously involved in the biosynthesis of natural products. However, P450s have proven to be difficult to express in host cells such as E. coli, thus limiting the amount of P450-catalyzed product produced by the host cell.

There is a need in the art for host cells that provide for improved expression and/or activity of P450 enzymes.

### Literature

Ro et al. (2005) Nature 440:940-943.

### SUMMARY OF THE INVENTION

The present invention provides genetically modified host cells that exhibit modified activity levels of one or more gene products such that, when a cytochrome P450 enzyme is produced in the genetically modified host cell, the modified activity levels of the one or more gene products provide for enhanced production and/or activity of the cytochrome P450 enzyme. The present invention provides methods of producing a cytochrome P450 enzyme in a host cell, generally involving culturing a subject genetically modified host cell in a suitable culture medium. The present invention further provides methods of producing a product of a P450-dependent oxidation, generally involving culturing a subject genetically modified host cell in a suitable culture medium.

### BRIEF DESCRIPTION OF THE DRAWINGS

FIGS. 1A and 1B depict measurements of the transcriptional response of E. coli to P450 expression and turnover.

FIGS. 2A and 2B depict a comparison of transcripts in amorphadiene oxidase (AMO) strains.

FIGS. 3A and 3B depict the effect of chaperone co-expression on AMO in vivo productivity.

FIGS. 4A and 4B depict nucleotide sequences encoding Artemisia annua amorphadiene oxidase (AMO).

FIG. 5 depicts a nucleotide sequence encoding A13-AMO.

FIG. 6 is a schematic representation of isoprenoid metabolic pathways that result in the production of the isoprenoid biosynthetic pathway intermediates polyprenyl diphosphates geranyl diphosphate (GPP), farnesyl diphosphate (FPP), and geranylgeranyl diphosphate (GGPPP), from isopentenyl diphosphate (IPP) and dimethylallyl diphosphate (DMAPP).

FIG. 7 is a schematic representation of the mevalonate (MEV) pathway for the production of IPP.

FIG. 8 is a schematic representation of the DXP pathway for the production of IPP and dimethylallyl pyrophosphate (DMAPP).

FIG. 9 depicts the effect of co-expression of various oxidative stress-related genes on amorphadiene oxidase turnover.

FIG. 10 is a schematic depiction of plasmid pAM92.

### DEFINITIONS

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “naturally-occurring” as used herein as applied to a nucleic acid, a cell, or an organism, refers to a nucleic acid, cell, or organism that is found in nature. For example, a polypeptide or polynucleotide sequence that is present in an organism (including viruses) that can be isolated from a source in nature and which has not been intentionally modified by a human in the laboratory is naturally occurring.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

As used herein, the term “exogenous nucleic acid” refers to a nucleic acid that is not normally or naturally found in and/or produced by a given bacterium, organism, or cell in nature. As used herein, the term “endogenous nucleic acid” refers to a nucleic acid that is normally found in and/or produced by a given bacterium, organism, or cell in nature. An “endogenous nucleic acid” is also referred to as a “native nucleic acid” or a nucleic acid that is “native” to a given bacterium, organism, or cell.

The term “heterologous nucleic acid,” as used herein, refers to a nucleic acid wherein at least one of the following is true: (a) the nucleic acid is foreign (“exogenous”) to (i.e., not naturally found in) a given host microorganism or host cell; (b) the nucleic acid comprises a nucleotide sequence that is naturally found in (e.g., is “endogenous to”) a given host microorganism or host cell (e.g., the nucleic acid comprises a nucleotide sequence that is endogenous to the host microorganism or host cell) but is either produced in an unnatural (e.g., greater than expected or greater than naturally found) amount in the cell, or differs in sequence from the endogenous nucleotide sequence such that the same encoded protein (having the same or substantially the same amino acid sequence) as found endogenously is produced in an unnatural (e.g., greater than expected or greater than naturally found) amount in the cell; (c) the nucleic acid comprises two or more nucleotide sequences or segments that are not found in the same relationship to each other in nature, e.g., the nucleic acid is recombinant.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below).

Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant” polypeptide refers to a polypeptide which is not naturally occurring, e.g., is made by the artificial combination of two otherwise separated segments of amino sequence through human intervention. Thus, e.g., a polypeptide that comprises a heterologous amino acid sequence is recombinant.

By “construct” or “vector” is meant a recombinant nucleic acid, generally recombinant DNA, which has been generated for the purpose of the expression and/or propagation of a specific nucleotide sequence(s), or is to be used in the construction of other recombinant nucleotide sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (i.e., DNA exogenous to the cell). Genetic change (“modification”) can be accomplished either by incorporation of the new DNA into the genome of the host cell, or by transient or stable maintenance of the new DNA as an episomal element. Where the cell is a eukaryotic cell, a permanent genetic change is generally achieved by introduction of the DNA into the genome of the cell. In prokaryotic cells, permanent changes can be introduced into the chromosome or via extrachromosomal elements such as plasmids and expression vectors, which may contain one or more selectable markers to aid in their maintenance in the recombinant host cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

A “host cell,” as used herein, denotes an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector that comprises a nucleotide sequence encoding one or more biosynthetic pathway gene products such as mevalonate pathway gene products), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject prokaryotic host cell is a genetically modified prokaryotic host cell (e.g., a bacterium), by virtue of introduction into a suitable prokaryotic host cell of a heterologous nucleic acid, e.g., an exogenous nucleic acid that is foreign to (not normally found in nature in) the prokaryotic host cell, or a recombinant nucleic acid that is not normally found in the prokaryotic host cell; and a subject eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

The terms “isoprenoid,” “isoprenoid compound,” “terpene,” “terpene compound,” “terpenoid,” and “terpenoid compound” are used interchangeably herein, and refer to any compound that is capable of being derived from isopentenyl pyrophosphate (IPP). The number of C-atoms present in the isoprenoids is typically evenly divisible by five (e.g., C5, C10, C15, C20, C25, C30 and C40). Irregular isoprenoids and polyterpenes have been reported, and are also included in the definition of “isoprenoid.” Isoprenoid compounds include, but are not limited to, monoterpenes, diterpenes, triterpenes, sesquiterpenes, and polyterpenes.

As used herein, the term “prenyl diphosphate” is used interchangeably with “prenyl pyrophosphate,” and includes monoprenyl diphosphates having a single prenyl group (e.g., IPP and DMAPP), as well as polyprenyl diphosphates that include 2 or more prenyl groups. Monoprenyl diphosphates include isopentenyl pyrophosphate (IPP) and its isomer dimethylallyl pyrophosphate (DMAPP).

As used herein, the term “terpene synthase” refers to any enzyme that enzymatically modifies IPP, DMAPP, or a polyprenyl pyrophosphate, such that a terpenoid precursor compound is produced. The term “terpene synthase” includes enzymes that catalyze the conversion of a prenyl diphosphate into an isoprenoid or isoprenoid precursor.

The word “pyrophosphate” is used interchangeably herein with “diphosphate.” Thus, e.g., the terms “prenyl diphosphate” and “prenyl pyrophosphate” are interchangeable; the terms “isopentenyl pyrophosphate” and “isopentenyl diphosphate” are interchangeable; the terms farnesyl diphosphate” and farnesyl pyrophosphate” are interchangeable; etc.

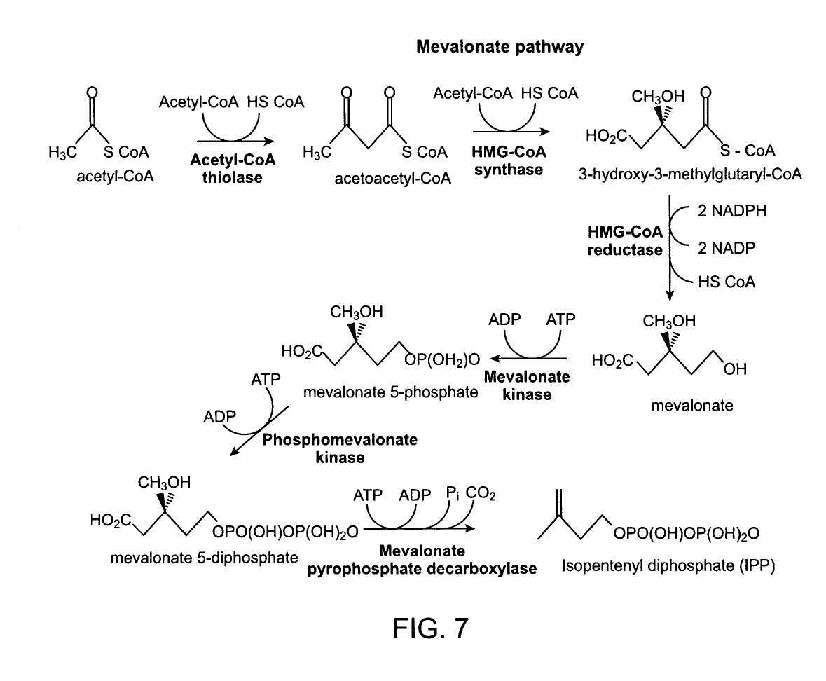

The term “mevalonate pathway” or “MEV pathway” is used herein to refer to the biosynthetic pathway that converts acetyl-CoA to IPP. The mevalonate pathway comprises enzymes that catalyze the following steps: (a) condensing two molecules of acetyl-CoA to acetoacetyl-CoA (e.g., by action of acetoacetyl-CoA thiolase); (b) condensing acetoacetyl-CoA with acetyl-CoA to form hydroxymethylglutaryl-CoenzymeA (HMG-CoA) (e.g., by action of HMG-CoA synthase (HMGS)); (c) converting HMG-CoA to mevalonate (e.g., by action of HMG-CoA reductase (HMGR)); (d) phosphorylating mevalonate to mevalonate 5-phosphate (e.g., by action of mevalonate kinase (MK)); (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)). The mevalonate pathway is illustrated schematically in FIG. 7. The “top half” of the mevalonate pathway refers to the enzymes responsible for the conversion of acetyl-CoA to mevalonate.

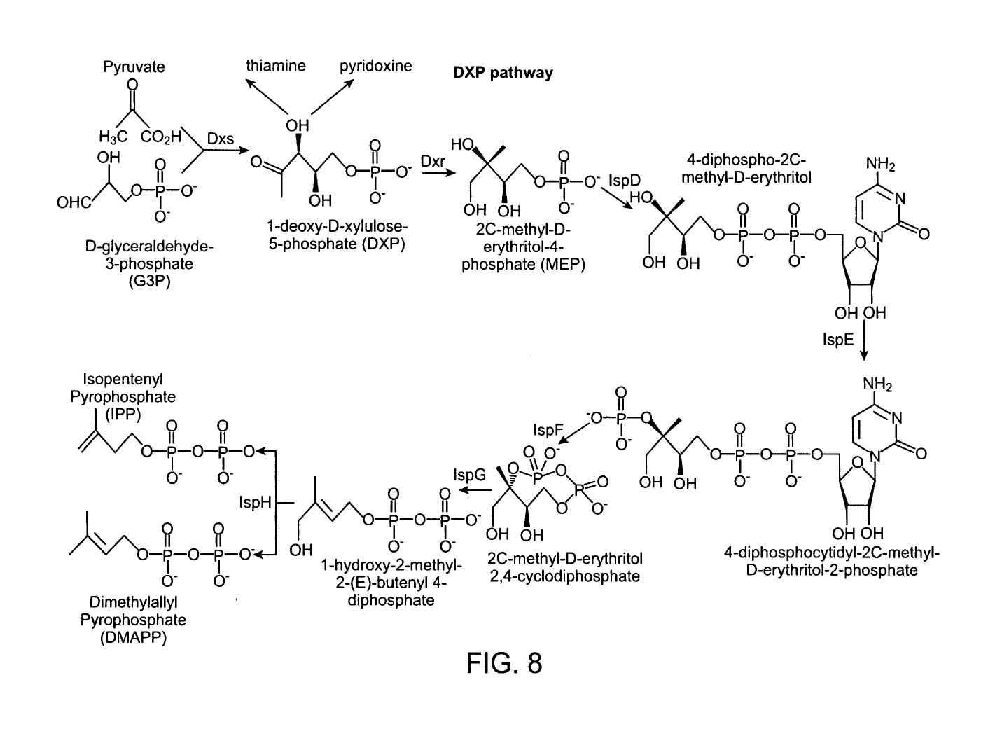

The term “1-deoxy-D-xylulose 5-diphosphate pathway” or “DXP pathway” is used herein to refer to the pathway that converts glyceraldehyde-3-phosphate and pyruvate to IPP and DMAPP through a DXP pathway intermediate, where DXP pathway comprises enzymes that catalyze the reactions depicted schematically in FIG. 8. Dxs is 1-deoxy-D-xylulose-5-phosphate synthase; Dxr is 1-deoxy-D-xylulose-5-phosphate reductoisomerase (also known as IspC); IspD is 4-diphosphocytidyl-2C-methyl-D-erythritol synthase; IspE is 4-diphosphocytidyl-2C-methyl-D-erythritol synthase; IspF is 2C-methyl-D-erythritol 2,4-cyclodiphosphate synthase; IspG is 1-hydroxy-2-methyl-2-(E)-butenyl 4-diphosphate synthase (IspG); and ispH is isopentenyl/dimethylallyl diphosphate synthase.

As used herein, the term “prenyl transferase” is used interchangeably with the terms “isoprenyl diphosphate synthase” and “polyprenyl synthase” (e.g., “GPP synthase,” “FPP synthase,” “OPP synthase,” etc.) to refer to an enzyme that catalyzes the consecutive 1′-4 condensation of isopentenyl diphosphate with allylic primer substrates, resulting in the formation of prenyl diphosphates of various chain lengths.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cytochrome P450 enzyme” includes a plurality of such enzymes and reference to “the P450-catalyzed modification product” includes reference to one or more such products and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides genetically modified host cells that exhibit modified activity levels of one or more gene products such that, when a cytochrome P450 enzyme is produced in the genetically modified host cell, the modified activity levels of the one or more gene products provide for enhanced production and/or activity of the cytochrome P450 enzyme. The present invention provides methods of producing a cytochrome P450 enzyme in a host cell, generally involving culturing a subject genetically modified host cell in a suitable culture medium. The present invention further provides methods of producing a product of a P450-catalyzed modification, generally involving culturing a subject genetically modified host cell in a suitable culture medium.

The chemical conversions carried out by cytochrome P450s (P450s) have substrate (oxygen) and cofactor (heme, iron, and NADPH) requirements that are general across the entire superfamily. In addition, P450s share many other similarities that may place a burden on the cell, such as the potential release of hydrogen peroxide during the catalytic cycle or membrane insertion/targeting. It has now been found that modulation of the levels of certain gene products in a host cell can result in improved P450 activity levels in the host cell. Such gene products include those involved in: a) cofactor biosynthesis or regeneration and nutrient assimilation; b) oxidative stress response; c) protein folding; d) heat shock response; e) osmotic stress response; f) low temperature growth; and g) transcriptional regulation of genes involved in oxidative stress or heat shock response.

### Genetically Modified Host Cells

The present invention provides genetically modified host cells that exhibit modified activity levels of one or more gene products, where the modified activity levels of the one or more gene products provide for enhanced production and/or activity of a cytochrome P450 enzyme in the cell. Modified activity levels of the one or more gene products can provide for enhanced production and/or activity of a cytochrome P450 enzyme in various ways. For example, modified activity levels of the one or more gene products can provide for one or more of: a) improved cell growth; b) reduced metabolic stress related to P450 turnover; c) increased level of a P450 polypeptide on a per cell basis; d) increased level of a P450 polypeptide on a per cell culture basis; and e) increased specific activity of a P450 enzyme. Enhanced production and/or activity of a cytochrome P450 can be on a per cell basis or on a per cell culture basis (e.g., on a per volume cell culture or per cell mass basis). Improved cell growth can lead to increased levels of P450 polypeptide (e.g., on a per cell culture basis) and/or increased specific activity of a P450 enzyme. Similarly, reduced metabolic stress related to P450 turnover can lead to increased levels of a P450 polypeptide and/or increased specific activity of a P450 enzyme. Increased production and/or activity of a cytochrome P450 can provide for increased production, on a per cell basis or on a per unit volume cell culture basis or on a cell mass basis, of one or more downstream products of the cytochrome P450 (e.g., a product of a P450-catalyzed modification (a “P450-catalyzed modification product”) and/or a downstream product of a P450-catalyzed modification product).

In some embodiments, a subject genetically modified host cell is further genetically modified with a nucleic acid comprising a nucleotide sequence encoding a cytochrome P450 enzyme, e.g., a heterologous nucleic acid comprising a nucleotide sequence encoding a cytochrome P450 enzyme. In some embodiments, a subject genetically modified host cell is further genetically modified with a nucleic acid comprising a nucleotide sequence encoding a cytochrome P450 reductase.

A cytochrome P450 enzyme catalyzes the modification of a biosynthetic pathway intermediate. In some embodiments, a subject genetically modified host cell is further genetically modified with one or more nucleic acids comprising nucleotide sequences encoding one or more enzymes that provide for production of a biosynthetic pathway intermediate that is a P450 substrate. In some embodiments, a subject genetically modified host cell is further genetically modified with one or more nucleic acids comprising nucleotide sequences encoding one or more enzymes that further modify a P450-catalyzed modification product.

A subject genetically modified host cell is useful for producing a P450, where the activity level of the P450 produced in a subject genetically modified host cell is higher than the activity level of the P450 produced in a control host cell. For example, the activity level of a P450 produced in a subject genetically modified host cell is at least about 10%, at least about 20%, at least about 25%, at least about 30%, at least about 40%, at least about 50%, at least about 60%, at least about 70%, at least about 80%, at least about 90%, at least about 100% (or two-fold), at least about 2.5-fold, at least about 3-fold, at least about 5-fold, at least about 7-fold, at least about 10-fold, at least about 15-fold, at least about 20-fold, at least about 50-fold, at least about 10²-fold, at least about 500-fold, or at least about 10³-fold, or more, higher than the activity level of the P450 in a control host cell. Increased activity levels of a P450 can be due to increased levels of the P450 protein and/or increased specific activity of the P450.

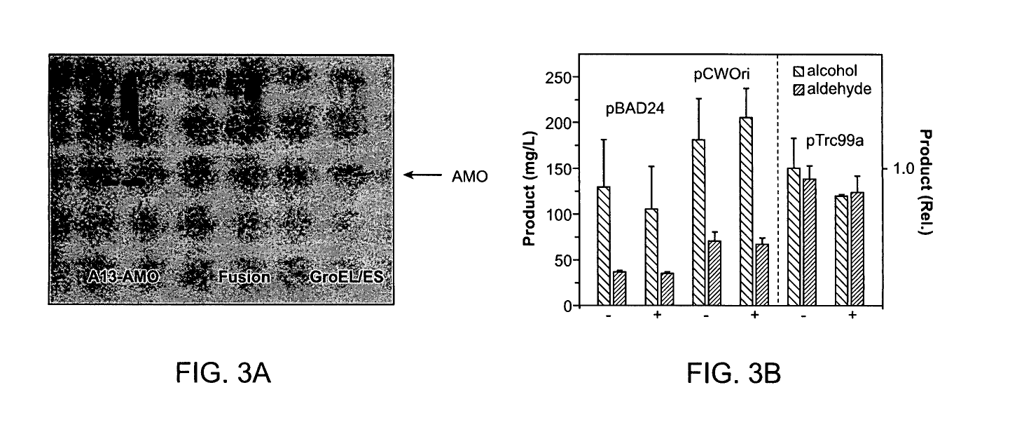

A cytochrome P450 enzyme produced in a subject genetically modified host cell catalyzes one or more of the following reactions: hydroxylation, oxidation, epoxidation, dehydration, dehydrogenation, dehalogenation, isomerization, alcohol oxidation, aldehyde oxidation, dealkylation, and C—C bond cleavage. Such reactions are referred to generically herein as “biosynthetic pathway intermediate modifications” or “P450-catalyzed modifications.” These reactions have been described in, e.g., Sono et al. ((1996) Chem. Rev. 96:2841-2887; see, e.g., FIG. 3 of Sono et al. for a schematic representation of such reactions).

In some embodiments, a subject genetically modified host cell is useful for producing a product of a P450-catalyzed modification (a “P450-catalyzed modification product”) and/or a downstream product of a P450-catalyzed modification product. In some embodiments, the P450-catalyzed modification product is one that is not normally produced by a control host cell, e.g., the P450-catalyzed modification product (or a downstream product thereof) is an exogenous product. In other embodiments, the P450-catalyzed modification product is one that is normally produced by the host cell, but is produced by a subject genetically modified host cell in amounts that are greater than the amount that would be produced by a control host cell. For example, in some embodiments, a P450-catalyzed modification product produced by a subject genetically modified host cell is produced in an amount that is at least about 10%, at least about 20%, at least about 25%, at least about 30%, at least about 40%, at least about 50%, at least about 60%, at least about 70%, at least about 80%, at least about 90%, at least about 100% (or two-fold), at least about 2.5-fold, at least about 3-fold, at least about 5-fold, at least about 7-fold, at least about 10-fold, at least about 15-fold, at least about 20-fold, at least about 50-fold, at least about 10²-fold, at least about 500-fold, at least about 10³-fold, at least about 5×10³-fold, or at least about 10⁴-fold, or more, higher than the amount of the product produced in a control host cell, on a per cell basis or on a per cell culture (e.g., unit cell culture volume) basis or on a per cell mass (e.g., per 10⁶ cells) basis. An example of a suitable control cell is a cell that is not genetically modified with a nucleic acid comprising a nucleotide sequence encoding a P450 activity enhancing gene product. For example, where a genetically modified host cell comprises: 1) a nucleic acid comprising a nucleotide sequence encoding a cytochrome P450 activity enhancing gene product; 2) a nucleic acid comprising a nucleotide sequence encoding a cytochrome P450 enzyme, e.g., a heterologous nucleic acid comprising a nucleotide sequence encoding a cytochrome P450 enzyme; and 3) one or more nucleic acids comprising nucleotide sequences encoding one or more enzymes that provide for production of a biosynthetic pathway intermediate that is a substrate of the cytochrome P450 enzyme, a suitable control cell is one that is genetically modified with: 1) the nucleic acid comprising a nucleotide sequence encoding a cytochrome P450 enzyme, e.g., a heterologous nucleic acid comprising a nucleotide sequence encoding a cytochrome P450 enzyme; and 2) the one or more nucleic acids comprising nucleotide sequences encoding one or more enzymes that provide for production of a biosynthetic pathway intermediate that is a substrate of the cytochrome P450 enzyme, but not with the nucleic acid comprising a nucleotide sequence encoding a cytochrome P450 activity enhancing gene product.

In some embodiments, a P450-catalyzed modification product produced by a subject genetically modified host cell is produced in an amount of from about 10 mg/L to about 50 g/L, e.g., from about 10 mg/L to about 25 mg/L, from about 25 mg/L to about 50 mg/L, from about 50 mg/L to about 75 mg/L, from about 75 mg/L to about 100 mg/L, from about 100 mg/L to about 250 mg/L, from about 250 mg/L to about 500 mg/L, from about 500 mg/L to about 750 mg/L, from about 750 mg/L to about 1000 mg/L, from about 1 g/L to about 1.2 g/L, from about 1.2 g/L to about 1.5 g/L, from about 1.5 g/L to about 1.7 g/L, from about 1.7 g/L to about 2 g/L, from about 2 g/L to about 2.5 g/L, from about 2.5 g/L to about 5 g/L, from about 5 g/L to about 10 g/L, from about 10 g/L to about 20 g/L, from about 20 g/L to about 30 g/L, from about 30 g/L to about 40 g/L, or from about 40 g/L to about 50 g/L, or more, on a cell culture basis.

In some embodiments, a subject genetically modified host cell comprises a nucleic acid comprising a nucleotide sequence encoding an oxidative stress-related gene product, wherein production of the oxidative stress-related gene product provides for increased production of an isoprenoid or isoprenoid precursor by the genetically modified host cell, compared to a control host cell not genetically modified with the nucleic acid. In some embodiments, the oxidative stress-related gene product is selected from glutamate-cysteine ligase and glutathione synthetase, δ-aminolevulinic acid synthase, and suf operon-encoded gene products. In some embodiments, the genetically modified host cell is genetically modified with a nucleic acid comprising nucleotide sequences encoding mevalonate pathway enzymes heterologous to the host cell; and the control host cell is genetically modified with the nucleic acid comprising nucleotide sequences encoding mevalonate pathway enzymes heterologous to the host cell, but not with the nucleic acid comprising a nucleotide sequence encoding an oxidative stress-related gene product.

In some embodiments, a subject genetically modified host cell comprises nucleic acid(s) comprising nucleotide sequences encoding mevalonate pathway enzymes, and is genetically modified with a nucleic acid(s) comprising a nucleotide sequence encoding a P450 enhancing gene product (e.g., is genetically modified with a nucleic acid comprising a nucleotide sequence encoding glutamate-cysteine ligase and glutathione synthetase, or δ-aminolevulinic acid synthase, or suf operon-encoded polypeptides); and a control host cell comprises the nucleic acid(s) comprising nucleotide sequences encoding mevalonate pathway enzymes; and is not genetically modified with the nucleic acid(s) comprising a nucleotide sequence encoding a P450 enhancing gene product. For example, in some embodiments, a subject genetically modified host cell comprises nucleic acid(s) comprising nucleotide sequences encoding mevalonate pathway enzymes that are heterologous to the host cell, and is genetically modified with a nucleic acid(s) comprising a nucleotide sequence encoding a P450 enhancing gene product (e.g., is genetically modified with a nucleic acid comprising a nucleotide sequence encoding glutamate-cysteine ligase and glutathione synthetase, or δ-aminolevulinic acid synthase, or suf operon-encoded polypeptides); and a control host cell comprises the nucleic acid(s) comprising nucleotide sequences encoding mevalonate pathway enzymes heterologous to the host cell; and is not genetically modified with the nucleic acid(s) comprising a nucleotide sequence encoding a P450 enhancing gene product. As one example, in some embodiments, a subject genetically modified host cell comprises a nucleic acid(s) comprising nucleotide sequences encoding acetoacetyl-CoA thiolase, HMGS, HMGR, MK, PMK, and MPD (e.g., SEQ ID NO:7 of U.S. Pat. No. 7,192,751), and is genetically modified with a nucleic acid(s) comprising a nucleotide sequence encoding a P450 enhancing gene product (e.g., is genetically modified with a nucleic acid comprising a nucleotide sequence encoding glutamate-cysteine ligase and glutathione synthetase, or δ-aminolevulinic acid synthase, or suf operon-encoded polypeptides); and a control host cell comprises the nucleic acid comprising nucleotide sequences encoding acetoacetyl-CoA thiolase, HMGS, HMGR, MK, PMK, and MPD (e.g., SEQ ID NO:7 of U.S. Pat. No. 7,192,751); and is not genetically modified with the nucleic acid(s) comprising a nucleotide sequence encoding a P450 enhancing gene product. As another example, in some embodiments, a subject genetically modified host cell comprises a nucleic acid(s) comprising nucleotide sequences encoding the “bottom half” of a mevalonate pathway (e.g., MK, PMK, and MPD; e.g., SEQ ID NO:9 of U.S. Pat. No. 7,192,751), and is genetically modified with a nucleic acid(s) comprising a nucleotide sequence encoding a P450 enhancing gene product (e.g., is genetically modified with a nucleic acid comprising a nucleotide sequence encoding glutamate-cysteine ligase and glutathione synthetase, or δ-aminolevulinic acid synthase, or suf operon-encoded polypeptides); and a control host cell comprises the nucleic acid comprising nucleotide sequences encoding MK, PMK and MPD, and is not genetically modified with the nucleic acid(s) comprising a nucleotide sequence encoding a P450 enhancing gene product. As another example, in some embodiments, a subject genetically modified host cell comprises a nucleic acid(s) comprising nucleotide sequences encoding MK, PMK, MPD, and isopententyl pyrophosphate isomerase (idi) (e.g., SEQ ID NO:12 of U.S. Pat. No. 7,192,751), and is genetically modified with a nucleic acid(s) comprising a nucleotide sequence encoding a P450 enhancing gene product (e.g., is genetically modified with a nucleic acid comprising a nucleotide sequence encoding glutamate-cysteine ligase and glutathione synthetase, or δ-aminolevulinic acid synthase, or suf operon-encoded polypeptides); and a control host cell comprises the nucleic acid comprising nucleotide sequences encoding MK, PMK, MPD, and idi, and is not genetically modified with the nucleic acid(s) comprising a nucleotide sequence encoding a P450 enhancing gene product. As another example, in some embodiments, a subject genetically modified host cell comprises a nucleic acid(s) comprising nucleotide sequences encoding MK, PMK, MPD, idi, and an FPP synthase (e.g., SEQ ID NO:13 of U.S. Pat. No. 7,192,751; e.g., SEQ ID NO:4 of U.S. Pat. No. 7,183,089), and is genetically modified with a nucleic acid(s) comprising a nucleotide sequence encoding a P450 enhancing gene product (e.g., is genetically modified with a nucleic acid comprising a nucleotide sequence encoding glutamate-cysteine ligase and glutathione synthetase, or δ-aminolevulinic acid synthase, or suf operon-encoded polypeptides); and a control host cell comprises the nucleic acid comprising nucleotide sequences encoding MK, PMK, MPD, idi, and an FPP synthase, and is not genetically modified with the nucleic acid(s) comprising a nucleotide sequence encoding a P450 enhancing gene product.

As one non-limiting example, in some embodiments, a subject genetically modified host cell comprises pAM92 (SEQ ID NO:70), and is genetically modified with a nucleic acid(s) comprising a nucleotide sequence encoding a P450 enhancing gene product (e.g., is genetically modified with a nucleic acid comprising a nucleotide sequence encoding glutamate-cysteine ligase and glutathione synthetase, or δ-aminolevulinic acid synthase, or suf operon-encoded polypeptides); and a control host cell comprises pAM92, and is not genetically modified with the nucleic acid(s) comprising a nucleotide sequence encoding a P450 enhancing gene product.

As one non-limiting example, in some embodiments, a subject genetically modified host cell comprises pAM92 (SEQ ID NO:70), and is genetically modified with a nucleic acid comprising a nucleotide sequence having at least about 75%, at least about 80%, at least about 85%, at least about 90%, at least about 95%, at least about 98%, at least about 99%, or 100%, nucleotide sequence identity to the P450 enhancing gene product-encoding nucleotide sequence set forth in SEQ ID NO:71, where the P450 enhancing gene product-encoding nucleotide sequence is operably linked to a promoter (e.g., an inducible promoter); and a control host cell comprises pAM92, and is not genetically modified with the nucleic acid comprising a nucleotide sequence encoding a P450 enhancing gene product.

As one non-limiting example, in some embodiments, a subject genetically modified host cell comprises pAM92 (SEQ ID NO:70), and is genetically modified with a nucleic acid comprising a nucleotide sequence having at least about 75%, at least about 80%, at least about 85%, at least about 90%, at least about 95%, at least about 98%, at least about 99%, or 100%, nucleotide sequence identity to the P450 enhancing gene product-encoding nucleotide sequence set forth in SEQ ID NO:20, where the P450 enhancing gene product-encoding nucleotide sequence is operably linked to a promoter (e.g., an inducible promoter); and a control host cell comprises pAM92, and is not genetically modified with the nucleic acid comprising a nucleotide sequence encoding a P450 enhancing gene product.

As one non-limiting example, in some embodiments, a subject genetically modified host cell comprises pAM92 (SEQ ID NO:70), and is genetically modified with a nucleic acid comprising a nucleotide sequence having at least about 75%, at least about 80%, at least about 85%, at least about 90%, at least about 95%, at least about 98%, at least about 99%, or 100%, nucleotide sequence identity to the P450 enhancing gene product-encoding nucleotide sequence set forth in SEQ ID NO:73, where the P450 enhancing gene product-encoding nucleotide sequence is operably linked to a promoter (e.g., an inducible promoter); and a control host cell comprises pAM92, and is not genetically modified with the nucleic acid comprising a nucleotide sequence encoding a P450 enhancing gene product.

### P450 Activity Enhancing Gene Products

As noted above, a subject genetically modified host cell exhibits modified activity levels of one or more gene products such that, when a cytochrome P450 enzyme is produced in the genetically modified host cell, the modified activity levels of the one or more gene products provide for enhanced production and/or activity of the cytochrome P450 enzyme. A gene product (e.g., an mRNA, a polypeptide, etc.) whose activity level, when modified, provides for enhanced production and/or activity of a cytochrome P450 enzyme in a subject genetically modified host cell, is referred to herein as a “P450 activity enhancing gene product.”

A P450 activity enhancing gene product increases one or both of: a) the amount of a P450 in a subject genetically modified host cell; b) an enzymatic activity of a P450 in a subject genetically modified host cell. For example, in some embodiments, the specific activity of a P450 is increased in a subject genetically modified host cell, compared to a control host cell. In some embodiments, the total amount of a P450 polypeptide in the cell is reduced, but the specific activity of the P450 is increased, compared to a control host cell. In other embodiments, both the total amount of a P450 and the specific activity of the P450 are increased.

Gene products whose activity levels, when modulated, provide for enhanced production and/or activity of a P450 in a subject genetically modified host cell include those involved in: a) cofactor biosynthesis or regeneration and nutrient assimilation; b) oxidative stress response; c) protein folding; d) heat shock response; e) osmotic stress response; f) low temperature growth; and g) transcriptional regulation of genes involved in oxidative stress or heat shock response. The following are non-limiting examples of such gene products.

Examples of gene products involved in co-factor biosynthesis or regeneration or in nutrient assimilation include gene products involved in NADPH biosynthesis; carbon assimilation via the pentose pathway; glutathione assimilation; sulfur assimilation; iron assimilation; and heme biosynthesis. Suitable NADPH biosynthesis and pentose phosphate pathway gene products include, but are not limited to, zwf, glucose-6-phosphate-1-dehydrogenase; pgl, 6-phosphogluconolactonase; gnd, 6-phosphogluconate dehydrogenase; and tktA, sedoheptulose-phosphate:glyceraldehyde-3-phosphate transketolase. Exemplary nucleotide sequences encoding NADPH and pentose phosphate pathway gene products are set forth in SEQ ID NOs: 1-4, where SEQ ID NO: 1 is a Escherichia coli glucose 6-phosphate-1-dehydrogenase-encoding nucleotide sequence; SEQ ID NO:2 is a E. coli 6-phosphogluconolactonase nucleotide sequence; SEQ ID NO:3 is a E. coli 6-phosphogluconate dehydrogenase-encoding nucleotide sequence; and SEQ ID NO:4 is a E. coli sedoheptulose-7-phosphate:glyceraldehyde-3-phosphate transketolase-encoding nucleotide sequence.

Suitable gene products involved in glutathione assimilation include, but are not limited to, gshAB, glutathione synthetase; gshB, glutathione synthetase; and Gor, glutathione reductase. Exemplary nucleotide sequences encoding glutathione assimilation gene products set forth in SEQ ID NOs:5-7, where SEQ ID NO:5 is a E. coli γ-glutamylcysteine synthetase-encoding nucleotide sequence; SEQ ID NO:6 is a E. coli glutathione synthase-encoding nucleotide sequence; and SEQ ID NO:7 is a E. coli glutathione reductase-encoding nucleotide sequence.

Suitable gene products involved in sulfur metabolism include, but are not limited to, cysA, cyst, cysW, cysP, sfp, tauA, tauB, tauC, fliY, cysDN, sulfate adenylyltransferase; and cysN. Exemplary nucleotide sequences encoding sulfur metabolism gene products are set forth in SEQ ID NOs:8-18, where SEQ ID NOs: 8, 9, 10, 11, and 12 are E. coli CysATWP-Sbp sulfate and thiosulfate ABC transporter-encoding nucleotide sequences, i.e., SEQ ID NOs: 8, 9, 10, 11, and 12 are E. coli cysA, cysT, cysW, cysP, and sfp, respectively; where SEQ ID NOs:13-15 are E. coli tauABC:taurin ABC transporter-encoding nucleotide sequences, i.e., SEQ ID NOs:13-15 are E. coli tauA, tauB, and tauC, respectively; where SEQ ID NO:16 is an E. coli fliY:cysteine transporter-encoding nucleotide sequence; and where SEQ ID NOs: 17 and 18 are E. coli cysDN:sulfate adenylyltransferase-encoding nucleotide sequences, i.e., SEQ ID NO:17 is E. coli cysD and SEQ ID NO:18 is E. coli cysN.

Suitable gene products involved in heme biosynthesis include, but are not limited to, hemA, glutamyl-tRNA reductase; hemA, 5-aminolevulinic acid synthase; and hemG, protoporphyrin oxidase. Exemplary nucleotide sequences encoding gene products involved in heme biosynthesis are set forth in SEQ ID NOs: 19-21, where SEQ ID NO: 19 is an E. coli hemA (glutamyl-tRNA reductase)-encoding nucleotide sequence; SEQ ID NO:20 is an Rhodobacter capsulatus δ-aminolevulinic acid (ALA) synthase-encoding nucleotide sequence; and SEQ ID NO:21 is an E. coli hemG:protoporphyrin oxidase-encoding nucleotide sequence.

Suitable gene products involved in iron metabolism include, but are not limited to, ytfE, iron metabolism protein; and hmpA, ferrisiderophore reductase or nitric oxide dehydrogenase. Exemplary nucleotide sequences encoding gene products involved in iron metabolism are set forth in SEQ ID NOs:22 and 23, where SEQ ID NO:22 is an E. coli ytfE:iron metabolism protein-encoding nucleotide sequence; and SEQ ID NO:23 is an E. coli hmpA:ferrisiderophore reductase or nitric oxide dehydrogenase-encoding nucleotide sequence.

Examples of gene products involved in oxidative stress response include, but are not limited to, gene products involved in one or more of: a) reactive oxygen species removal, where reactive oxygen species include, e.g., hydrogen peroxide, superoxide, and nitric oxide; b) repair of oxidative damage; c) Fe—S cluster assembly; d) repair of lipid peroxides; glutathione/glutaredoxin-dependent disulfide reduction; and e) maintenance of cellular redox potential. Suitable gene products involved in oxidative stress response include, but are not limited to, genes involved in hydrogen peroxide disproportionation, e.g., katG, catalase; and katE, catalase, where exemplary nucleotide sequences encoding such gene products are set forth in SEQ ID NOs:24 and 25, where SEQ ID NO:24 is an E. coli katG:catalase-encoding nucleotide sequence; and SEQ ID NO:25 is an E. coli katE:catalase-encoding nucleotide sequence. Suitable gene products involved in superoxide disproportionation include, but are not limited to, sodA, superoxide dismutase; and sodB, superoxide dismutase, where exemplary nucleotide sequences encoding such gene products are set forth in SEQ ID NOs:26 and 27, where SEQ ID NO:26 is an E. coli soda:superoxide dismutase-encoding nucleotide sequence; and SEQ ID NO:27 is an E. coli sodB:superoxide dismutase-encoding nucleotide sequence. Suitable gene products involved in repair of lipid peroxides include, but are not limited to, ahpCF, alkyl hydroperoxide reductase, where exemplary nucleotide sequences encoding such a gene product are set forth in SEQ ID NOs:28 and 29, encoding an E. coli ahpCF:alkyl hydroperoxide reductase, where SEQ ID NO:28 is an E. coli ahpC nucleotide sequence; and SEQ ID NO:29 is an E. coli ahpF nucleotide sequence. Suitable gene products involved in protein disulfide oxidation/reduction include, but are not limited to, grxA, glutaredoxin1; trxC, thioredoxin2; and ybbN, protein disulfide isomerase, where exemplary nucleotide sequences encoding such gene products are set forth in SEQ ID NOs:30-32, where SEQ ID NO:30 is an E. coli grxA:glutaredoxin1-encoding nucleotide sequence; SEQ ID NO:31 is an E. coli trxC:thioredoxin2-encoding nucleotide sequence; and SEQ ID NO:32 is an E. coli ybbn:protein disulfide isomerase-encoding nucleotide sequence.

Suitable gene products involved in Fe—S cluster repair and/or biosynthesis include, but are not limited to, sufA, Fe—S cluster assembly protein; sufBCD, cysteine desulfurase activator complex; sufc; sufD; sufS, cysteine desulfurase; sufE, cysteine desulfurase sulfur acceptor; iscS, cysteine desulfurase; iscU, Fe—S cluster assembly protein; and hscB, Fe—S cluster assembly chaperone, where exemplary nucleotide sequences encoding such gene products are set forth in SEQ ID NOs:33-42, where SEQ ID NO:33 is an E. coli sufA:Fe—S cluster assembly protein-encoding nucleotide sequence; SEQ ID NOs:34-36 are E. coli sufBCD:cysteine desulfurase activator complex-encoding nucleotide sequences, e.g., SEQ ID NO:34 is an E. coli sufB nucleotide sequence, SEQ ID NO:35 is an E. coli sufC nucleotide sequence, and SEQ ID NO:36 is an E. coli sufD nucleotide sequence; where SEQ ID NO:37 is an E. coli sufS:cysteine desulfurase-encoding nucleotide sequence; SEQ ID NO:38 is an E. coli sufE:cysteine desulfurase sulfur acceptor-encoding nucleotide sequence; SEQ ID NO:39 is an E. coli iscS:cysteine desulfurase-encoding nucleotide sequence; SEQ ID NO:40 is an E. coli iscU:Fe—S cluster assembly protein-encoding nucleotide sequence; SEQ ID NO:41 is an E. coli hscA:Fe—S cluster assembly chaperone-encoding nucleotide sequence; and SEQ ID NO:42 is an E. coli hscB:Fe—S cluster assembly chaperone-encoding nucleotide sequence.

Examples of gene products involved in protein folding or heat shock response include, but are not limited to, protein chaperones; heat shock proteins; gene products involved in modulation of transcription/translation activity; and proteases. Suitable gene products that are protein folding chaperones or are involved in heat shock response include, but are not limited to, groES/groEL, protein chaperone system; dnaKJ-GrpE, protein chaperone system; clpB, protein chaperone; ipbA, heat shock protein; ipbB, heat shock protein; and tig, peptidyl prolyl isomerase, where exemplary nucleotide sequences encoding such gene products are set forth in SEQ ID NOs:43-51, where SEQ ID NOs:43 and 44 are E. coli groES/groEL:protein chaperone system-encoding nucleotide sequence, e.g., SEQ ID NO:43 is an E. coli groES nucleotide sequence, and SEQ ID NO:44 is an E. coli groEL nucleotide sequence; SEQ ID NOs:45-47 are E. coli dnaKJ-GrpE:protein chaperone system-encoding nucleotide sequences, e.g., SEQ ID NO:45 is an E. coli dnaK nucleotide sequence, SEQ ID NO:46 is an E. coli dnaJ nucleotide sequence, and SEQ ID NO:47 is an E. coli grpE nucleotide sequence; SEQ ID NO:48 is an E. coli clpB:protein chaperone-encoding nucleotide sequence; SEQ ID NO:49 is an E. coli ipbA:heat shock protein-encoding nucleotide sequence; SEQ ID NO:50 is an E. coli ipbB:heat shock protein-encoding nucleotide sequence; and SEQ ID NO:51 is an E. coli tig:peptidyl prolyl isomerase-encoding nucleotide sequence.

Suitable protease gene products include, but are not limited to, hslVU, heat-shock related protease complex, where exemplary nucleotide sequences encoding such gene products are seq forth in SEQ ID NOs:52 and 53, encoding E. coli hslVU:heat-shock related protease complex, where SEQ ID NO:52 is an E. coli hslV nucleotide sequence, and SEQ ID NO:53 is an E. coli hslU nucleotide sequence.

Examples of gene products involved in response to osmotic stress and/or low temperature growth include, but are not limited to, transporters; gene products involved in biosynthesis of molecules used to maintain osmotic pressure; gene products involved in biosynthesis of molecules used to aid in low temperature growth; and genes involved in osmotically-regulated oxidative stress response. Suitable gene products involved in response to osmotic stress and/or low temperature growth conditions include, but are not limited to, proVWX, proline ABC transporter; otsA, trehalose-6-phosphate synthase; otsB, trehalose-6-phosphate phosphatase; betA, choline dehydrogenase; betB betaine aldehyde hydrogenase; betT, choline transporter; and osmC, osmoticaly-induced peroxidase, where exemplary nucleotide sequences encoding such gene products are set forth in SEQ ID NOs:54-62, where SEQ ID NOs:54-56 are E. coli proVWX:proline ABC transporter-encoding nucleotide sequences, e.g., SEQ ID NO:54 is an E. coli proV nucleotide sequence, SEQ ID NO:55 is an E. coli proW nucleotide sequence, and SEQ ID NO:56 is an E. coli proX nucleotide sequence; where SEQ ID NO:57 is an E. coli otsA:trehalose-6-phosphate synthase-encoding nucleotide sequence; where SEQ ID NO:58 is an E. coli otsB:trehalose-6-phosphate phosphatase-encoding nucleotide sequence; where SEQ ID NO:59 is an E. coli betA:choline dehydrogenase-encoding nucleotide sequence; where SEQ ID NO:60 is an E. coli betB:betaine aldehyde hydrogenase-encoding nucleotide sequence; where SEQ ID NO:61 is an E. coli betT:choline transporter-encoding nucleotide sequence; and where SEQ ID NO:62 is an E. coli osmC:osmotically-induced peroxidase-encoding nucleotide sequence.

Examples of gene products that are transcriptional regulators include, but are not limited to, transcriptional regulators of oxidative stress response genes; and transcriptional regulators of heat shock response genes. Suitable gene products include, but are not limited to, oxyR, peroxide stress transcriptional regulator; soxS, superoxide stress transcriptional regulator; marA, oxidative stress transcriptional regulator; and rpoH, heat shock response transcriptional regulator, where exemplary nucleotide sequences encoding such gene products are set forth in SEQ ID NOs:63-66, where SEQ ID NO:63 is an E. coli oxyR:peroxide stress-encoding nucleotide sequence; where SEQ ID NO:64 is an E. coli soxS:superoxide stress-encoding nucleotide sequence; where SEQ ID NO:65 is an E. coli marA:oxidative stress-encoding v; and where SEQ ID NO:66 is an E. coli rpoH:heat shock response-encoding nucleotide sequence.

In some embodiments, a suitable nucleotide sequence encoding a P450 activity enhancing gene product has at least about 75%, at least about 80%, at least about 85%, at least about 90%, at least about 95%, at least about 98%, at least about 99%, or 100%, nucleotide sequence identity to the nucleotide sequence set forth in any one of SEQ ID NOs: 1-66, e.g., a suitable nucleotide sequence encoding a P450 activity enhancing gene product has at least about 75%, at least about 80%, at least about 85%, at least about 90%, at least about 95%, at least about 98%, at least about 99%, or 100%, nucleotide sequence identity over the entire length of the nucleotide sequence set forth in any one of SEQ ID NOs: 1-66. In some embodiments, the nucleotide sequence includes, at the 5′ end of the sequence, a ribosome binding site.

In some embodiments, a suitable nucleotide sequence encoding a P450 activity enhancing gene product having at least about 75%, at least about 80%, at least about 85%, at least about 90%, at least about 95%, at least about 98%, at least about 99%, or 100%, nucleotide sequence identity to the nucleotide sequence set forth in any one of SEQ ID NOs:1-66, is codon optimized for expression in Escherichia coli.

For example, in some embodiments, a suitable nucleotide sequence encoding a P450 activity enhancing gene product is a nucleotide sequence encoding glutamate-cysteine ligase (e.g., gshA) and glutathione synthetase (e.g., gshB) activities. For example, in some embodiments, a suitable nucleotide sequence has at least about 75%, at least about 80%, at least about 85%, at least about 90%, at least about 95%, at least about 98%, at least about 99%, or 100%, nucleotide sequence identity to the nucleotide sequences set forth in SEQ ID NOs:5 and 6, where SEQ ID NO:5 is a nucleotide sequence encoding glutamate-cysteine ligase, and where SEQ ID NO:6 is a nucleotide sequence encoding a glutathione synthetase. In some embodiments, a suitable nucleotide sequence has at least about 75%, at least about 80%, at least about 85%, at least about 90%, at least about 95%, at least about 98%, at least about 99%, or 100%, nucleotide sequence identity to the nucleotide sequences set forth in SEQ ID NO:71, where SEQ ID NO:71 provides nucleotide sequences encoding glutamate-cysteine ligase (gshA) and glutathione synthase (gshB); where the coding regions are preceded by a ribosome binding site (RBS; AAGGAGATATACAT; SEQ ID NO:72); and where the glutamate-cysteine ligase coding sequence and the glutathione synthase coding sequence are separated by a cccggg restriction endonuclease recognition sequence followed by a RBS. In some embodiments, the start codon is ATG. GshA and GshB nucleotide sequences from a variety of organisms are known in the art. See, e.g., Vergauwen et al. (2006) J. Biol. Chem. 281:4380.

As another example, in some embodiments, a suitable nucleotide sequence encoding a P450 activity enhancing gene product is a nucleotide sequence encoding δ-aminolevulinic acid (ALA) synthase. For example, in some embodiments, a suitable nucleotide sequence has at least about 75%, at least about 80%, at least about 85%, at least about 90%, at least about 95%, at least about 98%, at least about 99%, or 100%, nucleotide sequence identity to the nucleotide sequence set forth in SEQ ID NO:20, where SEQ ID NO:20 is a Rhodobacter capsulatus ALA synthase-encoding nucleotide sequence. Other ALA synthase-encoding nucleotide sequences are known in the art. See, e.g., GenBank Accession No. CP000489 (Paracoccus denitrificans ALA synthase-encoding nucleotide sequence, encoding the amino acid sequence set forth in GenBank ABL69919); GenBank Accession No. CP000158 (Hyphomonas neptumium ALA synthase-encoding nucleotide sequence, encoding the amino acid sequence set forth in GenBank ABI76065.1); etc.

As another example, in some embodiments, a suitable nucleotide sequence encoding a P450 activity enhancing gene product is a nucleotide sequence encoding suf operon-encoded gene products. For example, in some embodiments, a suitable nucleotide sequence has at least about 75%, at least about 80%, at least about 85%, at least about 90%, at least about 95%, at least about 98%, at least about 99%, or 100%, nucleotide sequence identity to the nucleotide sequence set forth in SEQ ID NOs:33-38, collectively known as “suf operon,” where SEQ ID NO:33 (sufA) encodes an Fe—S cluster assembly protein, SEQ ID NOs:34-36 (sufBCD) encodes a cysteine desulfurase activator complex, SEQ ID NO:37 (sufS) encodes a cysteine desulfurase, and SEQ ID NO:38 (sufE) encodes a cysteine desulfurase sulfur acceptor. See Outten et al. (2004) Molec. Microbiol. 52:861 for a discussion of the suf operon in E. coli: Huet et al. (2005) J. Bacteriol. 187:6137 for a discussion of the suf operon in Mycobacterium tuberculosis. In some embodiments, a suitable nucleotide sequence has at least about 75%, at least about 80%, at least about 85%, at least about 90%, at least about 95%, at least about 98%, at least about 99%, or 100%, nucleotide sequence identity to the nucleotide sequence set forth in SEQ ID NO:73 (sufABCDSE).

### Modulating Levels of a P450 Activity Enhancing Gene Product

A subject genetically modified host cell is genetically modified so as to exhibit modified activity levels of one or more P450 activity enhancing gene products such that, when a cytochrome P450 enzyme is produced in the genetically modified host cell, the modified activity levels of the one or more P450 activity enhancing gene products provide for enhanced production and/or activity of the cytochrome P450 enzyme. “Modulating an activity level of a P450 activity enhancing gene product” includes increasing an activity level of a P450 activity enhancing gene product and decreasing an activity level of a P450 activity enhancing gene product. Increasing the activity level of a P450 activity enhancing gene product can be achieved by increasing the total amount of the P450 activity enhancing gene product in a cell; and/or increasing the activity of the P450 activity enhancing gene product. Similarly, decreasing the activity level of a P450 activity enhancing gene product can be achieved by decreasing the total amount of the P450 activity enhancing gene product; and/or decreasing the activity of the P450 activity enhancing gene product.

The activity level of a P450 activity enhancing gene product can be modulated in any of a number of ways, including, but not limited to, overexpressing the P450 activity enhancing gene product in the cell; downregulating expression of the P450 activity enhancing gene product in the cell; deleting a P450 activity enhancing gene product coding region; and mutating a P450 activity enhancing gene product, or a gene encoding the P450 activity enhancing gene product. Overexpressing a P450 activity enhancing gene product in a cell can be achieved by one or more of increasing the copy number of a nucleic acid that encodes the P450 activity enhancing gene product; and increasing the promoter strength of a promoter operably linked to a coding region encoding the P450 activity enhancing gene product.

The activity level of a P450 activity enhancing gene product can be increased in a number of ways, including, but not limited to, (1) increased transcription of a nucleic acid encoding the P450 activity enhancing gene product; 2) increased translation of an mRNA encoding the P450 activity enhancing gene product; 3) increased stability of the mRNA encoding the P450 activity enhancing gene product; 4) increased stability of the P450 activity enhancing gene product itself; and 5) altered specific activity (units activity per unit protein) of the P450 activity enhancing gene product. The level of transcription of a nucleic acid in a host cell can be increased in a number of ways, including, but not limited to, increasing the strength of the promoter (transcription initiation or transcription control sequence) to which the P450 activity enhancing gene product coding region is operably linked (for example, using a consensus arabinose- or lactose-inducible promoter in a prokaryotic host cell in place of a modified lactose-inducible promoter, such as the one found in pBluescript and the pBBR1MCS plasmids), increasing the copy number of the nucleotide sequence encoding the P450 activity enhancing gene product (for example, by using a higher copy number expression vector comprising a nucleotide sequence encoding the P450 activity enhancing gene product, or by introducing additional copies of a nucleotide sequence encoding the P450 activity enhancing gene product into the genome of the host cell, for example, by recA-mediated recombination, use of “suicide” vectors, recombination using lambda phage recombinase, and/or insertion via a transposon or transposable element), changing the order of the coding regions on the polycistronic mRNA of an operon or breaking up an operon into individual genes, each with its own control elements, or using an inducible promoter and inducing the inducible-promoter by adding a chemical to a growth medium. Increasing the relative activity level of a P450 activity enhancing gene product in a host cell can be achieved by increasing the number of copies in the host cell of nucleic acids encoding the P450 activity enhancing gene product, which nucleic acids can be integrated into the chromosome of the host cell or present as extra-chromosomal elements.

The level of translation of a nucleotide sequence encoding a gene product in a host cell can be altered in a number of ways, including, but not limited to, increasing the stability of the mRNA, modifying the sequence of the ribosome binding site, modifying the distance or sequence between the ribosome binding site and the start codon of the coding sequence, modifying the entire intercistronic region located “upstream of” or adjacent to the 5′ side of the start codon of the coding region, stabilizing the 3′-end of the mRNA transcript using hairpins and specialized sequences, modifying the codon usage, altering expression of rare codon tRNAs used in the biosynthesis of the gene product, and/or increasing the stability of the gene product, as, for example, via mutation of its coding sequence. Determination of preferred codons and rare codon tRNAs can be based on a survey of genes derived from the host cell.

In some embodiments, an expression vector comprising a nucleotide sequence encoding a P450 activity enhancing gene product is introduced into a host cell, to generate a genetically modified host cell, where expression vector provides for low, medium, or high copy number of the vector in the cell. In some embodiments, the expression vector is present in the genetically modified host cell at a level of about 10 copies, between 10 and 20 copies, between 20 and 50 copies, or between 50 and 100 copies, or greater than 100 copies per cell. Low copy number plasmids generally provide fewer than about 20 plasmid copies per cell; medium copy number plasmids generally provide from about 20 plasmid copies per cell to about 50 plasmid copies per cell, or from about 20 plasmid copies per cell to about 80 plasmid copies per cell; and high copy number plasmids generally provide from about 80 plasmid copies per cell to about 200 plasmid copies per cell, or more.

Suitable low copy expression vectors for prokaryotic cells such as Escherichia coli include, but are not limited to, pACYC184, pBeloBac11, pBR332, pBAD33, pBBR1MCS and its derivatives, pSC101, SuperCos (cosmid), and pWE15 (cosmid). Suitable medium copy expression vectors for Escherichia coli include, but are not limited to pTrc99A, pBAD24, and vectors containing a ColE1 origin of replication and its derivatives. Suitable high copy number expression vectors for prokaryotic cells such as Escherichia coli include, but are not limited to, pUC, pBluescript, pGEM, and pTZ vectors. Suitable low-copy (centromeric) expression vectors for yeast include, but are not limited to, pRS415 and pRS416 (Sikorski & Hieter (1989) Genetics 122:19-27). Suitable high-copy 2 micron expression vectors in yeast include, but are not limited to, pRS425 and pRS426 (Christainson et al. (1992) Gene 110:119-122). Alternative 2 micron expression vectors include non-selectable variants of the 2 micron vector (Bruschi & Ludwig (1988) Curr. Genet. 15:83-90) or intact 2 micron plasmids bearing an expression cassette (as exemplified in U.S. Pat. Publication No. 20050084972).

### P450 Nucleic Acids

A subject genetically modified host cell is genetically modified to provide for modulated activity levels of one or more P450 activity enhancing gene products; and in some embodiments is further genetically modified with a nucleic acid comprising a nucleotide sequence encoding a P450 enzyme. Amino acid sequences of a variety of P450 enzymes are known in the art, as are nucleotide sequences encoding the P450 enzymes. Suitable P450 enzymes include, but are not limited to, isoprenoid pathway intermediate-modifying P450s, alkaloid pathway intermediate-modifying P450s, phenylpropanoid pathway intermediate-modifying P450s, and polyketide pathway intermediate-modifying P450s.

The encoded cytochrome P450 enzyme will carry out one or more of the following reactions: hydroxylation, epoxidation, oxidation, dehydration, dehydrogenation, dehalogenation, isomerization, alcohol oxidation, aldehyde oxidation, dealkylation, and C—C bond cleavage. Such reactions are referred to generically herein as “biosynthetic pathway intermediate modifications”; and the products of such reaction as referred to herein as “P450 modification products.”

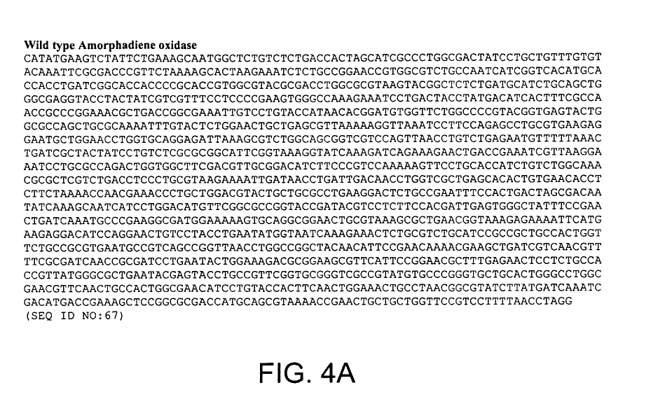

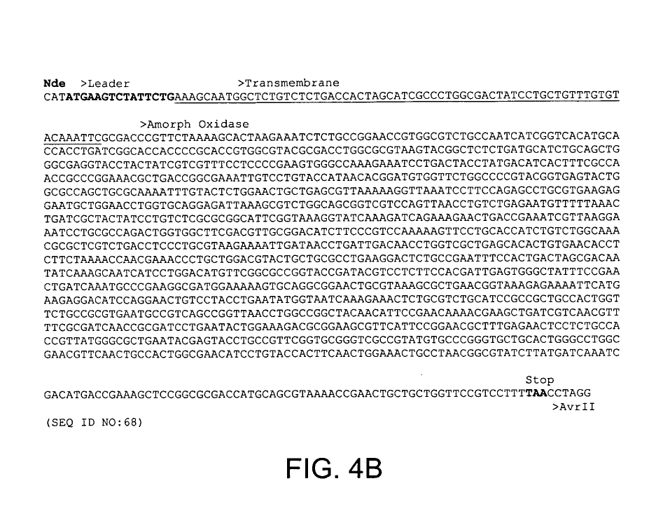

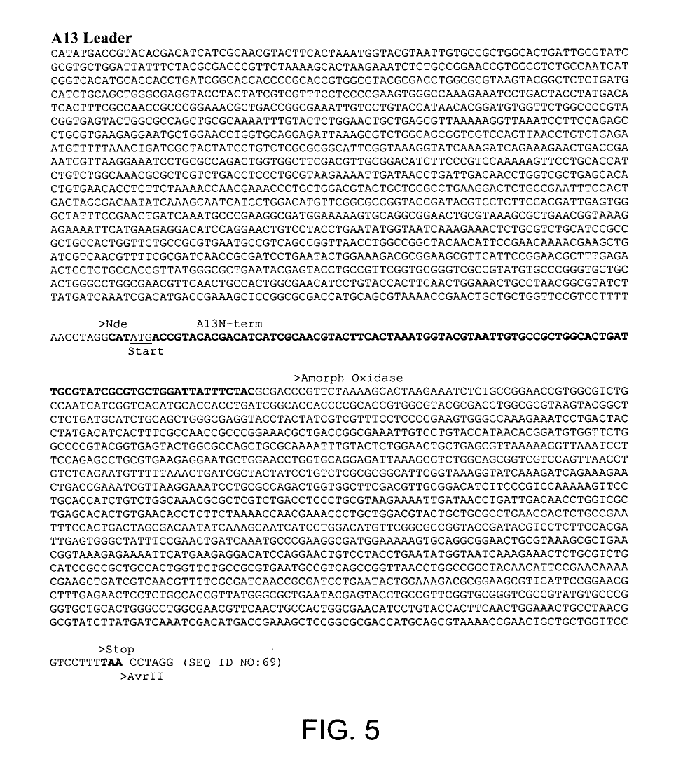

Suitable P450 enzymes include isoprenoid pathway intermediate-modifying P450s. Isoprenoid pathway intermediate-modifying P450s, include, but are not limited to, a limonene-6-hydroxylase (see, e.g., GenBank Accession Nos. AY281025 and AF124815); 5-epi-aristolochene dihydroxylase (see, e.g., GenBank Accession No. AF368376); 6-cadinene-8-hydroxylase (see, e.g., GenBank Accession No. AF332974); taxadiene-5α-hydroxylase (see, e.g., GenBank Accession Nos. AY289209, AY959320, and AY364469); ent-kaurene oxidase (see, e.g., GenBank Accession No. AF047719; see, e.g., Helliwell et al. (1998) Proc. Natl. Acad. Sci. USA 95:9019-9024); and amorphadiene oxidase. Exemplary amorphadiene oxidase (AMO) sequences are depicted in FIGS. 4A and 4B (Artemisia annua AMO); and FIG. 5 (A13-AMO, synthetic AMO codon optimized for expression in E. coli, with the wild-type transmembrane region replaced with A13 N-terminal sequence from C. tropicalis).

Suitable P450 enzymes include alkaloid pathway intermediate-modifying P450s. Alkaloid pathway intermediate-modifying cytochrome P450 enzymes are known in the art. See, e.g., Facchini et al. (2004) supra; Pauli and Kutchan ((1998) Plant J. 13:793-801; Collu et al. ((2001) FEBS Lett. 508:215-220; Schroder et al. ((1999) FEBS Lett. 458:97-102.

Suitable P450 enzymes include phenylpropanoid pathway intermediate-modifying P450s. Phenylpropanoid pathway intermediate-modifying cytochrome P450 enzymes are known in the art. See, e.g., Mizutani et al. ((1997) Plant Physiol. 113:755-763; and Gang et al. ((2002) Plant Physiol. 130:1536-1544.

Suitable P450 enzymes include polyketide pathway intermediate-modifying P450s. Polyketide pathway intermediate-modifying cytochrome P450 enzymes are known in the art. See e.g., Ikeda et al. ((1999) Proc. Natl. Acad. Sci. USA 96:9509-9514; and Ward et al. ((2004) Antimicrob. Agents Chemother. 48:4703-4712.

In some embodiments, the nucleotide sequence encoding a P450 enzyme encodes a P450 enzyme that has from about 50% to about 55%, from about 55% to about 60%, from about 60% to about 65%, from about 65% to about 70%, from about 70% to about 75%, from about 75% to about 80%, from about 80% to about 85%, from about 85% to about 90%, or from about 90% to about 95% amino acid sequence identity to the amino acid sequence of a naturally-occurring P450 enzyme.

In some embodiments, the P450 comprises one or more modifications relative to a wild-type P450. For example, in some embodiments, the modified cytochrome P450 enzyme will have a non-native (non-wild-type, or non-naturally occurring, or variant) amino acid sequence. In some embodiments, the modified cytochrome P450 enzyme will have one or more amino acid sequence modifications (deletions, additions, insertions, substitutions) that increase the level of activity of the modified cytochrome P450 enzyme.

The coding sequence of any known P450 may be altered in various ways known in the art to generate targeted changes in the amino acid sequence of the encoded enzyme, generating a variant P450. The amino acid sequence of a variant P450 will in some embodiments be substantially similar to the amino acid sequence of any known P450 enzyme, i.e. will differ by at least one amino acid, and may differ by at least two, at least 5, at least 10, or at least 20 amino acids, but not more than about fifty amino acids. The sequence changes may be substitutions, insertions or deletions. For example, the nucleotide sequence can be altered for the codon bias of a particular host cell. In addition, one or more nucleotide sequence differences can be introduced that result in conservative amino acid changes in the encoded P450 protein.

In some embodiments, a modified P450 comprises one or more of the following: a) substitution of a native transmembrane domain with a non-native transmembrane domain; b) replacement of the native transmembrane domain with a secretion signal domain; c) replacement of the native transmembrane domain with a solubilization domain; d) replacement of the native transmembrane domain with membrane insertion domain; e) truncation of the native transmembrane domain; and f) a change in the amino acid sequence of the native transmembrane domain.

For example, for expression in E. coli, suitable non-native transmembrane domain can comprise one of the following the amino acid sequences:

(SEQ ID NO:74)

NH₂-MWLLLIAVFLLTLAYLFWP-COOH;

(SEQ ID NO:75)

NH₂-MALLLAVFLGLSCLLLLSLW-COOH;

(SEQ ID NO:76)

NH₂-MAILAAIFALVVATATRV-COOH;

(SEQ ID NO:77)

NH₂-MDASLLLSVALAVVLIPLSLALLN-COOH;
and

(SEQ ID NO:78)

NH₂-MIEQLLEYWYVVVPVLYIIKQLLAYTK-COOH.

Secretion signals that are suitable for use in bacteria include, but are not limited to, the secretion signal of Braun's lipoprotein of E. coli, S. marcescens, E. amylosora, M. morganii, and P. mirabilis, the TraT protein of E. coli and Salmonella; the penicillinase (PenP) protein of B. lichenifonnis and B. cereus and S. aureus; pullulanase proteins of Klebsiella pneumoniae and Klebsiella aerogenese; E. coli lipoproteins 1pp-28, Pal, Rp1A, Rp1B, OsmB, NIpB, and Orl17; chitobiase protein of V. harseyi; the β-1,4-endoglucanase protein of Pseudomonas solanacearum, the Pal and Pcp proteins of H. influenzae; the OprI protein of P. aeruginosa; the MalX and AmiA proteins of S. pneumoniae; the 34 kda antigen and TpmA protein of Treponema pallidum; the P37 protein of Mycoplasma hyorhinis; the neutral protease of Bacillus amyloliquefaciens; the 17 kda antigen of Rickettsia rickettsii; the malE maltose binding protein; the rbsB ribose binding protein; phoA alkaline phosphatase; and the OmpA secretion signal (see, e.g., Tanji et al. (1991) J. Bacteriol. 173(6):1997-2005). Secretion signal sequences suitable for use in yeast are known in the art, and can be used. See, e.g., U.S. Pat. No. 5,712,113. The rbsB, malE, and phoA secretion signals are discussed in, e.g., Collier (1994) J. Bacteriol. 176:3013.

In some embodiments, e.g., for expression in a prokaryotic host cell such as E. coli, a secretion signal will comprise one of the following amino acid sequences:

| | |
|---|---|
| NH₂-MKKTAIAIAVALAGFATVAQA-COOH; | (SEQ ID NO:79) |
| NH₂-MKKTAIAIVVALAGFATVAQA-COOH; | (SEQ ID NO:80) |
| NH₂-MKKTALALAVALAGFATVAQA-COOH; | (SEQ ID NO:81) |
| NH₂-MKIKTGARILALSALTTMMFSASALA-COOH; | (SEQ ID NO:82) |
| NH₂-MNMKKLATLVSAVALSATVSANAMA-COOH; | (SEQ ID NO:83) |
| and | |
| NH₂-MKQSTIALALLPLLFTPVTKA-COOH. | (SEQ ID NO:84) |

In some embodiments, the modified cytochrome P450 enzyme will comprise both a non-native secretion signal sequence and a heterologous transmembrane domain. Any combination of secretion signal sequence and heterologous transmembrane domain can be used.

In some embodiments, a solubilization domain will comprise one or more of the following amino acid sequences:

(SEQ ID NO:85)

NH₂-EELLKQALQQAQQLLQQAQELAKK-COOH;
and

(SEQ ID NO:86)

NH₂-MTVHDIIATYFTKWYVIVPLALIAYRVLDYFY-COOH;

(SEQ ID NO:87)

NH₂-GLFGAIAGFIEGGWTGMIDGWYGYGGGKK-COOH;
and

(SEQ ID NO:88)

NH₂-MAKKTSSKG-COOH.

In some embodiments, the modified cytochrome P450 enzyme will comprise a non-native amino acid sequence that provides for insertion into a membrane. In some embodiments, the modified cytochrome P450 enzyme is a fusion polypeptide that comprises a heterologous fusion partner (e.g., a protein other than a cytochrome P450 enzyme) fused in-frame at either the amino terminus or the carboxyl terminus, where the fusion partner provides for insertion of the fusion protein into a biological membrane.

In some embodiments, the fusion partner is a mistic protein, e.g., a protein comprising the amino acid sequence depicted in GenBank Accession No. AY874162. A nucleotide sequence encoding the mistic protein is also provided under GenBank Accession No. AY874162. Other polypeptides that provide for insertion into a biological membrane are known in the art and are discussed in, e.g., PsbW Woolhead et al. (J. Biol. Chem. 276 (18): 14607), describing PsbW; and Kuhn (FEMS Microbiology Reviews 17 (1992i) 285), describing M12 procoat protein and Pf3 procoat protein.

### Cytochrome P450 Reductase

NADPH-cytochrome P450 oxidoreductase (CPR, EC 1.6.2.4) is the redox partner of many P450-monooxygenases. In some embodiments, a subject genetically modified host cell further comprises a nucleic acid comprising a nucleotide sequence encoding a cytochrome P450 reductase (CPR). A nucleic acid comprising a nucleotide sequence encoding a CPR is referred herein to as “a CPR nucleic acid.” A CPR encoded by a CPR nucleic acid transfers electrons from NADPH to a cytochrome P450 enzyme.

In some embodiments, a nucleic acid comprises a nucleotide sequence encoding both a cytochrome P450 enzyme and a CPR. In some embodiments, a nucleic acid comprises a nucleotide sequence encoding a fusion protein that comprises an amino acid sequence of cytochrome P450 enzyme fused to a CPR polypeptide. In some embodiments, the encoded fusion protein is of the formula NH₂-A-X—B—COOH, where A is the cytochrome P450 enzyme, X is an optional linker, and B is the CPR polypeptide. In some embodiments, the encoded fusion protein is of the formula NH₂-A-X—B—COOH, where A is the CPR polypeptide, X is an optional linker, and B is the cytochrome P450 enzyme.

The linker peptide may have any of a variety of amino acid sequences. Proteins can be joined by a spacer peptide, generally of a flexible nature, although other chemical linkages are not excluded. The linker may be a cleavable linker. Suitable linker sequences will generally be peptides of between about 5 and about 50 amino acids in length, or between about 6 and about 25 amino acids in length. Peptide linkers with a degree of flexibility will generally be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use according to the present invention.

In some embodiments, a nucleic acid comprises a nucleotide sequence encoding a CPR polypeptide that has at least about 45%, at least about 50%, at least about 55%, at least about 57%, at least about 60%, at least about 65%, at least about 70%, at least about 75%, at least about 80%, at least about 85%, at least about 90%, at least about 95%, at least about 98%, or at least about 99% amino acid sequence identity to a known or naturally-occurring CPR polypeptide.

The coding sequence of any known CPR may be altered in various ways known in the art to generate targeted changes in the amino acid sequence of the encoded CPR, generating a variant CPR. The amino acid sequence of a variant CPR will in some embodiments be substantially similar to the amino acid sequence of any known CPR, i.e. will differ by at least one amino acid, and may differ by at least two, at least 5, at least 10, or at least 20 amino acids, but not more than about fifty amino acids. The sequence changes may be substitutions, insertions or deletions. For example, the nucleotide sequence can be altered for the codon bias of a particular host cell. In addition, one or more nucleotide sequence differences can be introduced that result in conservative amino acid changes in the encoded CPR protein,

CPR polypeptides, as well as nucleic acids encoding the CPR polypeptides, are known in the art, and any CPR-encoding nucleic acid, or a variant thereof, can be used in the instant invention. Suitable CPR-encoding nucleic acids include nucleic acids encoding CPR found in plants. Suitable CPR-encoding nucleic acids include nucleic acids encoding CPR found in fungi. Examples of suitable CPR-encoding nucleic acids include: GenBank Accession No. AJ303373 (Triticum aestivum CPR); GenBank Accession No. AY959320 (Taxus chinensis CPR); GenBank Accession No. AY532374 (Ammi majus CPR); GenBank Accession No. AG211221 (Oryza sativa CPR); and GenBank Accession No. AF024635 (Petroselinum crispum CPR); Candida tropicalis cytochrome P450 reductase (GenBank Accession No. M35199); Arabidopsis thaliana cytochrome P450 reductase ATR1 (GenBank Accession No. X66016); and Arabidopsis thaliana cytochrome P450 reductase ATR2 (GenBank Accession No. X66017); and putidaredoxin reductase and putidaredoxin (GenBank Accession No. J05406).

In some embodiments, a nucleic acid comprises a nucleotide sequence that encodes a CPR polypeptide that is specific for a given P450 enzyme. As one non-limiting example, a subject nucleic acid comprises a nucleotide sequence that encodes Taxus cuspidata CPR (GenBank AY571340). As another non-limiting example, a subject nucleic acid comprises a nucleotide sequence that encodes Candida tropicalis CPR. In other embodiments, a subject nucleic acid comprises a nucleotide sequence that encodes a CPR polypeptide that can serve as a redox partner for two or more different P450 enzymes. One such CPR is Arabidopsis thaliana cytochrome P450 reductase (ATR1). Another such CPR is Arabidopsis thaliana cytochrome P450 reductase (ATR2).

### Biosynthetic Pathway Enzymes

As noted above, in some embodiments, a subject genetically modified host cell is further genetically modified with one or more nucleic acids comprising nucleotide sequences encoding one or more enzymes that provide for production of a biosynthetic pathway intermediate that is a P450 substrate. In some embodiments, a subject genetically modified host cell is further genetically modified with one or more nucleic acids comprising nucleotide sequences encoding one or more enzymes that further modify a P450 modification product.

In some embodiments, the one or more enzymes that provide for production of a biosynthetic pathway intermediate that is a P450 substrate are enzymes that provide for production of an isoprenoid or an isoprenoid precursor (e.g., isopentenyl pyrophosphate (IPP), mevalonate, etc.). In these embodiments, the P450 is an isoprenoid precursor-modifying enzyme. The term “isoprenoid precursor-modifying P450 enzyme,” used interchangeably herein with “isoprenoid-modifying P450 enzyme,” refers to a P450 enzyme that modifies an isoprenoid precursor compound, e.g., with an isoprenoid precursor compound as substrate, the isoprenoid precursor-modifying P450 enzyme catalyzes one or more of the following reactions: hydroxylation, epoxidation, oxidation, dehydration, dehydrogenation, dehalogenation, isomerization, alcohol oxidation, aldehyde oxidation, dealkylation, and C—C bond cleavage. Such reactions are referred to generically herein as “P450-catalyzed isoprenoid precursor modifications.”

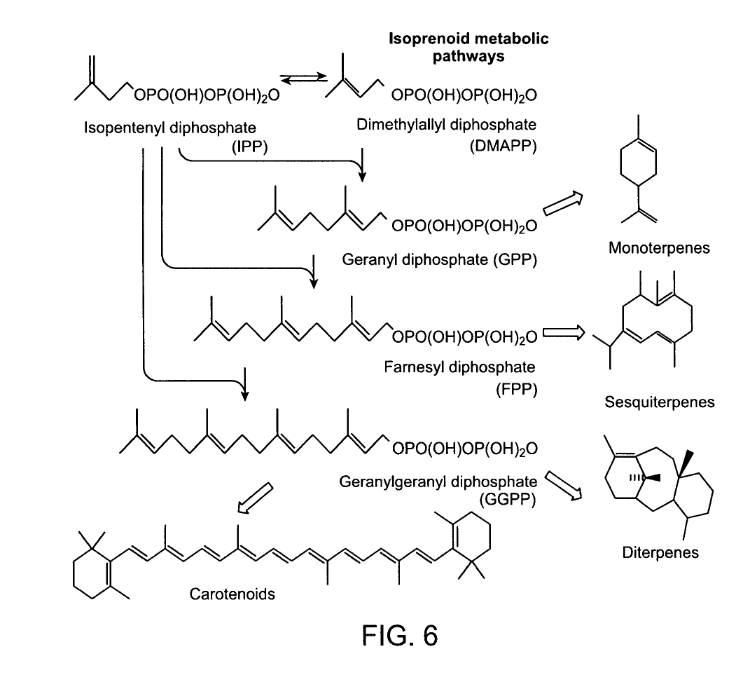

FIG. 6 depicts isoprenoid pathways involving modification of isopentenyl diphosphate (IPP) and/or its isomer dimethylallyl diphosphate (DMAPP) by prenyl transferases to generate the polyprenyl diphosphates geranyl diphosphate (GPP), farnesyl diphosphate (FPP), and geranylgeranyl diphosphate (GGPP). GPP and FPP are further modified by terpene synthases to generate monoterpenes and sesquiterpenes, respectively; and GGPP is further modified by terpene synthases to generate diterpenes and carotenoids. IPP and DMAPP are generated by one of two pathways: the mevalonate (MEV) pathway and the 1-deoxy-D-xylulose-5-phosphate (DXP) pathway.

FIG. 7 depicts schematically the MEV pathway, where acetyl CoA is converted via a series of reactions to IPP.

FIG. 8 depicts schematically the DXP pathway, in which pyruvate and D-glyceraldehyde-3-phosphate are converted via a series of reactions to IPP and DMAPP. Eukaryotic cells other than plant cells use the MEV isoprenoid pathway exclusively to convert acetyl-coenzyme A (acetyl-CoA) to IPP, which is subsequently isomerized to DMAPP. Plants use both the MEV and the mevalonate-independent, or DXP pathways for isoprenoid synthesis. Prokaryotes, with some exceptions, use the DXP pathway to produce IPP and DMAPP separately through a branch point.

Examples of enzymes that provide for production of isoprenoid or isoprenoid precursor that is a substrate for an isoprenoid-modifying P450 include, but are not limited to terpene synthases; prenyl transferases; isopentenyl diphosphate isomerase; one or more enzymes in a mevalonate pathway; and one or more enzymes in a DXP pathway. In some embodiments, a subject genetically modified host cell is further genetically modified to include one or more nucleic acids comprising nucleotide sequences encoding one, two, three, four, five, six, seven, eight, or more of: a terpene synthase, a prenyl transferase, an IPP isomerase, an acetoacetyl-CoA thiolase, a hydroxymethyl glutaryl-CoA synthase (HMGS), a hydroxymethyl glutaryl-CoA reductase (HMGR), a mevalonate kinase (MK), a phosphomevalonate kinase (PMK), and a mevalonate pyrophosphate decarboxylase (MPD). In some embodiments, e.g., where a subject genetically modified host cell is further genetically modified to include one or more nucleic acids comprising nucleotide sequences encoding two or more of a terpene synthase, a prenyl transferase, an IPP isomerase, an acetoacetyl-CoA thiolase, an HMGS, an HMGR, an MK, a PMK, and an MPD, the nucleotide sequences are present in at least two operons, e.g., two separate operons, three separate operons, or four separate operons.

### Terpene Synthases

In some embodiments, a subject genetically modified host cell is further genetically modified to include a nucleic acid comprising a nucleotide sequence encoding a terpene synthase. In some embodiments, the terpene synthase is one that modifies FPP to generate a sesquiterpene. In other embodiments, the terpene synthase is one that modifies GPP to generate a monoterpene. In other embodiments, the terpene synthase is one that modifies GGPP to generate a diterpene. The terpene synthase acts on a polyprenyl diphosphate substrate, modifying the polyprenyl diphosphate substrate by cyclizing, rearranging, or coupling the substrate, yielding an isoprenoid precursor (e.g., limonene, amorphadiene, taxadiene, etc.), which isoprenoid precursor is the substrate for an isoprenoid precursor-modifying enzyme(s). By action of the terpene synthase on a polyprenyl diphosphate substrate, the substrate for an isoprenoid-precursor-modifying enzyme is produced.

Nucleotide sequences encoding terpene synthases are known in the art, and any known terpene synthase-encoding nucleotide sequence can be used to genetically modify a host cell. For example, the following terpene synthase-encoding nucleotide sequences, followed by their GenBank accession numbers and the organisms in which they were identified, are known and can be used: (−)-germacrene D synthase mRNA (AY438099; Populus balsamifera subsp. trichocarpa×Populus deltoids); E,E-alpha-farnesene synthase mRNA (AY640154; Cucumis sativus); 1,8-cineole synthase mRNA (AY691947; Arabidopsis thaliana); terpene synthase 5 (TPS5) mRNA (AY518314; Zea mays); terpene synthase 4 (TPS4) mRNA (AY518312; Zea mays); myrcene/ocimene synthase (TPS10) (At2g24210) mRNA (NM_{—}127982; Arabidopsis thaliana); geraniol synthase (GES) mRNA (AY362553; Ocimum basilicum); pinene synthase mRNA (AY237645; Picea sitchensis); myrcene synthase le20 mRNA (AY195609; Antirrhinum majus); (E)-β-ocimene synthase (0e23) mRNA (AY195607; Antirrhinum majus); E-β-ocimene synthase mRNA (AY151086; Antirrhinum majus); terpene synthase mRNA (AF497-492; Arabidopsis thaliana); (−)-camphene synthase (AG6.5) mRNA (U87910; Abies grandis); (−)-4S-limonene synthase gene (e.g., genomic sequence) (AF326518; Abies grandis); delta-selinene synthase gene (AF326513; Abies grandis); amorpha-4,11-diene synthase mRNA (AJ251751; Artemisia annua); E-α-bisabolene synthase mRNA (AF006195; Abies grandis); gamma-humulene synthase mRNA (U92267; Abies grandis); 6-selinene synthase mRNA (U92266; Abies grandis); pinene synthase (AG3.18) mRNA (U87909; Abies grandis); myrcene synthase (AG2.2) mRNA (U87908; Abies grandis); etc.

### Mevalonate Pathway

In some embodiments, a subject genetically modified host cell is a host cell that does not normally synthesize isopentenyl pyrophosphate (IPP) or mevalonate via a mevalonate pathway. The mevalonate pathway comprises: (a) condensing two molecules of acetyl-CoA to acetoacetyl-CoA; (b) condensing acetoacetyl-CoA with acetyl-CoA to form HMG-CoA; (c) converting HMG-CoA to mevalonate; (d) phosphorylating mevalonate to mevalonate 5-phosphate; (e) converting mevalonate 5-phosphate to mevalonate 5-pyrophosphate; and (f) converting mevalonate 5-pyrophosphate to isopentenyl pyrophosphate. The mevalonate pathway enzymes required for production of IPP vary, depending on the culture conditions.

As noted above, in some embodiments, a subject genetically modified host cell is a host cell that does not normally synthesize isopentenyl pyrophosphate (IPP) or mevalonate via a mevalonate pathway. In some of these embodiments, the host cell is genetically modified with an expression vector comprising a nucleic acid encoding an isoprenoid-modifying P450 enzyme; and the host cell is genetically modified with one or more heterologous nucleic acids comprising nucleotide sequences encoding acetoacetyl-CoA thiolase, hydroxymethylglutaryl-CoA synthase (HMGS), hydroxymethylglutaryl-CoA reductase (HMGR), mevalonate kinase (MK), phosphomevalonate kinase (PMK), and mevalonate pyrophosphate decarboxylase (MPD) (and optionally also IPP isomerase). In some of these embodiments, the host cell is genetically modified with an expression vector comprising a nucleotide sequence encoding a CPR. In some of these embodiments, the host cell is genetically modified with an expression vector comprising a nucleic acid encoding an isoprenoid-modifying P450 enzyme; and the host cell is genetically modified with one or more heterologous nucleic acids comprising nucleotide sequences encoding MK, PMK, MPD (and optionally also IPP isomerase). In some of these embodiments, the host cell is genetically modified with an expression vector comprising a nucleotide sequence encoding a CPR.

In some embodiments, a subject genetically modified host cell is a host cell that does not normally synthesize IPP or mevalonate via a mevalonate pathway; the host cell is genetically modified with an expression vector comprising a nucleic acid encoding an isoprenoid-modifying P450 enzyme; and the host cell is genetically modified with one or more heterologous nucleic acids comprising nucleotide sequences encoding acetoacetyl-CoA thiolase, HMGS, HMGR, MK, PMK, MPD, IPP isomerase, and a prenyl transferase. In some of these embodiments, the host cell is genetically modified with an expression vector comprising a nucleotide sequence encoding a CPR. In some embodiments, a subject genetically modified host cell is a host cell that does not normally synthesize IPP or mevalonate via a mevalonate pathway; the host cell is genetically modified with an expression vector comprising a nucleic acid encoding an isoprenoid-modifying P450 enzyme; and the host cell is genetically modified with one or more heterologous nucleic acids comprising nucleotide sequences encoding MK, PMK, MPD, IPP isomerase, and a prenyl transferase. In some of these embodiments, the host cell is genetically modified with an expression vector comprising a nucleotide sequence encoding a CPR.

In some embodiments, a subject genetically modified host cell is one that normally synthesizes IPP or mevalonate via a mevalonate pathway, e.g., the host cell is one that comprises an endogenous mevalonate pathway. In some of these embodiments, the host cell is a yeast cell. In some of these embodiments, the host cell is Saccharomyces cerevisiae.

### Mevalonate Pathway Nucleic Acids

Nucleotide sequences encoding MEV pathway gene products are known in the art, and any known MEV pathway gene product-encoding nucleotide sequence can used to generate a subject genetically modified host cell. For example, nucleotide sequences encoding acetoacetyl-CoA thiolase, HMGS, HMGR, MK, PMK, MPD, and IDI are known in the art. The following are non-limiting examples of known nucleotide sequences encoding MEV pathway gene products, with GenBank Accession numbers and organism following each MEV pathway enzyme, in parentheses: acetoacetyl-CoA thiolase: (NC_{—}000913 REGION: 2324131 . . . 2325315; E. coli), (D49362; Paracoccus denitrificans), and (L20428; Saccharomyces cerevisiae); HMGS: (NC_{—}001145. complement 19061 . . . 20536; Saccharomyces cerevisiae), (X96617; Saccharomyces cerevisiae), (X83882; Arabidopsis thaliana), (AB037907; Kitasatospora griseola), and (BT007302; Homo sapiens); HMGR: (NM_{—}206548; Drosophila melanogaster), (NM_{—}204485; Gallus gallus), (AB015627; Streptomyces sp. KO-3988), (AF542543; Nicotiana attenuata), (AB037907; Kitasatospora griseola), (AX128213, providing the sequence encoding a truncated HMGR; Saccharomyces cerevisiae), and (NC_{—}001145: complement (115734.118898; Saccharomyces cerevisiae)); MK: (L77688; Arabidopsis thaliana), and (X55875; Saccharomyces cerevisiae); PMK: (AF429385; Hevea brasiliensis), (NM_{—}006556; Homo sapiens), (NC_{—}001145. complement 712315 . . . 713670; Saccharomyces cerevisiae); MPD: (X97557; Saccharomyces cerevisiae), (AF290095; Enterococcus faecium), and (U49260; Homo sapiens); and IDI: (NC_{—}000913, 3031087 . . . 3031635; E. coli), and (AF082326; Haematococcus pluvialis).

In some embodiments, the HMGR coding region encodes a truncated form of HMGR (“tHMGR”) that lacks the transmembrane domain of wild-type HMGR. The transmembrane domain of HMGR contains the regulatory portions of the enzyme and has no catalytic activity.

In some embodiments, a nucleic acid comprises a nucleotide sequence encoding a MEV pathway enzyme that has at least about 45%, at least about 50%, at least about 55%, at least about 57%, at least about 60%, at least about 65%, at least about 70%, at least about 75%, at least about 80%, at least about 85%, at least about 90%, at least about 95%, at least about 98%, or at least about 99% amino acid sequence identity to a known or naturally-occurring MEV pathway enzyme.

The coding sequence of any known MEV pathway enzyme may be altered in various ways known in the art to generate targeted changes in the amino acid sequence of the encoded enzyme. The amino acid sequence of a variant MEV pathway enzyme will in some embodiments be substantially similar to the amino acid sequence of any known MEV pathway enzyme, i.e. will differ by at least one amino acid, and may differ by at least two, at least 5, at least 10, or at least 20 amino acids, but typically not more than about fifty amino acids. The sequence changes may be substitutions, insertions or deletions. For example, as described below, the nucleotide sequence can be altered for the codon bias of a particular host cell. In addition, one or more nucleotide sequence differences can be introduced that result in conservative amino acid changes in the encoded protein.

### Prenyl Transferases

In some embodiments, a subject genetically modified host cell is genetically modified to include a nucleic acid comprising a nucleotide sequence encoding an isoprenoid-modifying P450 enzyme; and in some embodiments is also genetically modified to include one or more nucleic acids comprising a nucleotide sequence(s) encoding one or more mevalonate pathway enzymes, as described above; and a nucleic acid comprising a nucleotide sequence that encodes a prenyl transferase.

Prenyltransferases constitute a broad group of enzymes catalyzing the consecutive condensation of IPP resulting in the formation of prenyl diphosphates of various chain lengths. Suitable prenyltransferases include enzymes that catalyze the condensation of IPP with allylic primer substrates to form isoprenoid compounds with from about 2 isoprene units to about 6000 isoprene units or more, e.g., 2 isoprene units (Geranyl Pyrophosphate synthase), 3 isoprene units (Farnesyl pyrophosphate synthase), 4 isoprene units (geranylgeranyl pyrophosphate synthase), 5 isoprene units, 6 isoprene units (hexadecylpyrophosphate synthase), 7 isoprene units, 8 isoprene units (phytoene synthase, octaprenyl pyrophosphate synthase), 9 isoprene units (nonaprenyl pyrophosphate synthase, 10 isoprene units (decaprenyl pyrophosphate synthase), from about 10 isoprene units to about 15 isoprene units, from about 15 isoprene units to about 20 isoprene units, from about 20 isoprene units to about 25 isoprene units, from about 25 isoprene units to about 30 isoprene units, from about 30 isoprene units to about 40 isoprene units, from about 40 isoprene units to about 50 isoprene units, from about 50 isoprene units to about 100 isoprene units, from about 100 isoprene units to about 250 isoprene units, from about 250 isoprene units to about 500 isoprene units, from about 500 isoprene units to about 1000 isoprene units, from about 1000 isoprene units to about 2000 isoprene units, from about 2000 isoprene units to about 3000 isoprene units, from about 3000 isoprene units to about 4000 isoprene units, from about 4000 isoprene units to about 5000 isoprene units, or from about 5000 isoprene units to about 6000 isoprene units or more.

Suitable prenyltransferases include, but are not limited to, an E-isoprenyl diphosphate synthase, including, but not limited to, geranyl diphosphate (GPP) synthase, farnesyl diphosphate (FPP) synthase, geranylgeranyl diphosphate (GGPP) synthase, hexaprenyl diphosphate (HexPP) synthase, heptaprenyl diphosphate (HepPP) synthase, octaprenyl (OPP) diphosphate synthase, solanesyl diphosphate (SPP) synthase, decaprenyl diphosphate (DPP) synthase, chicle synthase, and gutta-percha synthase; and a Z-isoprenyl diphosphate synthase, including, but not limited to, nonaprenyl diphosphate (NPP) synthase, undecaprenyl diphosphate (UPP) synthase, dehydrodolichyl diphosphate synthase, eicosaprenyl diphosphate synthase, natural rubber synthase, and other Z-isoprenyl diphosphate synthases.

The nucleotide sequences of a numerous prenyl transferases from a variety of species are known, and can be used or modified for use in generating a subject genetically modified host cell. Nucleotide sequences encoding prenyl transferases are known in the art. See, e.g., Human farnesyl pyrophosphate synthetase mRNA (GenBank Accession No. J05262; Homo sapiens); farnesyl diphosphate synthetase (FPP) gene (GenBank Accession No. J05091; Saccharomyces cerevisiae); isopentenyl diphosphate:dimethylallyl diphosphate isomerase gene (J05090; Saccharomyces cerevisiae); Wang and Ohnuma (2000) Biochim. Biophys. Acta 1529:33-48; U.S. Pat. No. 6,645,747; Arabidopsis thaliana farnesyl pyrophosphate synthetase 2 (FPS2)/FPP synthetase 2/farnesyl diphosphate synthase 2 (At4 g17190) mRNA (GenBank Accession No. NM_{—}202836); Ginkgo biloba geranylgeranyl diphosphate synthase (ggpps) mRNA (GenBank Accession No. AY371321); Arabidopsis thaliana geranylgeranyl pyrophosphate synthase (GGPS1)/GGPP synthetase/farnesyltranstransferase (At4g36810) mRNA (GenBank Accession No. NM_{—}119845); Synechococcus elongatus gene for farnesyl, geranylgeranyl, geranylfarnesyl, hexaprenyl, heptaprenyl diphosphate synthase (SelF-HepPS) (GenBank Accession No. AB016095); etc.

### Expression Constructs

A subject genetically modified host cell is generated by genetically modifying a parent cell to exhibit modified activity levels of one or more P450 activity enhancing gene products. As noted above, in some embodiments, a subject genetically modified host cell is further genetically modified with a nucleic acid comprising a nucleotide sequence encoding a cytochrome P450 enzyme. In some embodiments, a subject genetically modified host cell is further genetically modified with a nucleic acid comprising a nucleotide sequence encoding a cytochrome P450 reductase. In some embodiments, a subject genetically modified host cell is further genetically modified with one or more nucleic acids comprising nucleotide sequences encoding one or more enzymes that provide for production of a biosynthetic pathway intermediate that is a P450 substrate. In some embodiments, a subject genetically modified host cell is further genetically modified with one or more nucleic acids comprising nucleotide sequences encoding one or more enzymes that further modify a P450 modification product.

One or more heterologous nucleic acids comprising nucleotide sequences encoding one or more of: a) a P450 activity enhancing gene product(s); b) a P450; c) a CPR; d) one or more enzymes that provide for production of a biosynthetic pathway intermediate that is a P450 substrate; and e) one or more enzymes that further modify a P450 modification product, are introduced into a parent host cell, generating a genetically modified host cell. The one or more heterologous nucleic acids can be expression constructs that provide for production of the encoded gene product in the host cell. Expression constructs generally include one or more transcriptional control elements, and a selectable marker.

### Transcriptional Control Elements

Non-limiting examples of suitable eukaryotic promoters include CMV immediate early, HSV thymidine kinase, early and late SV40, LTRs from retrovirus, and mouse metallothionein-I. In some embodiments, e.g., for expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHO5 promoter, a CUP1 promoter, a GAL7 promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. The expression vector may also contain a ribosome binding site for translation initiation and a transcription terminator. The expression vector may also include appropriate sequences for amplifying expression.

In some embodiments, the promoter is an inducible promoter. In some embodiments, the promoter is a constitutive promoter. In yeast, a number of vectors containing constitutive or inducible promoters may be used. For a review see, Current Protocols in Molecular Biology, Vol. 2, 1988, Ed. Ausubel, et al., Greene Publish. Assoc. & Wiley Interscience, Ch. 13; Grant, et al., 1987, Expression and Secretion Vectors for Yeast, in Methods in Enzymology, Eds. Wu & Grossman, 31987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (Cloning in Yeast, Ch. 3, R. Rothstein In: DNA Cloning Vol. II, A Practical Approach, Ed. DM Glover, 1986, IRL Press, Wash., D.C.). Alternatively, vectors may be used which promote integration of foreign DNA sequences into the yeast chromosome.

In some embodiments, a promoter or other regulatory element(s) suitable for expression in a plant cell is used. Non-limiting examples of suitable constitutive promoters that are functional in a plant cell is the cauliflower mosaic virus 35S promoter, a tandem 35S promoter (Kay et al., Science 236:1299 (1987)), a cauliflower mosaic virus 19S promoter, a nopaline synthase gene promoter (Singer et al., Plant Mol. Biol. 14:433 (1990); An, Plant Physiol. 81:86 (1986), an octopine synthase gene promoter, and a ubiquitin promoter. Suitable inducible promoters that are functional in a plant cell include, but are not limited to, a phenylalanine ammonia-lyase gene promoter, a chalcone synthase gene promoter, a pathogenesis-related protein gene promoter, a copper-inducible regulatory element (Mett et al., Proc. Natl. Acad. Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Röder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992); a nitrate-inducible promoter derived from the spinach nitrite reductase gene (Back et al., Plant Mol. Biol. 17:9 (1991)); a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)); a light-responsive regulatory element as described in U.S. Patent Publication No. 20040038400; a salicylic acid inducible regulatory elements (Uknes et al., Plant Cell 5:159-169 (1993); Bi et al., Plant J. 8:235-245 (1995)); plant hormone-inducible regulatory elements (Yamaguchi-Shinozaki et al., Plant Mol. Biol. 15:905 (1990); Kares et al., Plant Mol. Biol. 15:225 (1990)); and human hormone-inducible regulatory elements such as the human glucocorticoid response element (Schena et al., Proc. Natl. Acad. Sci. USA 88:10421 (1991).

Plant tissue-selective regulatory elements also can be included in a subject nucleic acid or a subject vector. Suitable tissue-selective regulatory elements, which can be used to ectopically express a nucleic acid in a single tissue or in a limited number of tissues, include, but are not limited to, a xylem-selective regulatory element, a tracheid-selective regulatory element, a fiber-selective regulatory element, a trichome-selective regulatory element (see, e.g., Wang et al. (2002) J. Exp. Botany 53:1891-1897), a glandular trichome-selective regulatory element, and the like.

Vectors that are suitable for use in plant cells are known in the art, and any such vector can be used to introduce a subject nucleic acid into a plant host cell. Suitable vectors include, e.g., a Ti plasmid of Agrobacterium tumefaciens or an Ri₁ plasmid of A. rhizogenes. The Ti or Ri₁ plasmid is transmitted to plant cells on infection by Agrobacterium and is stably integrated into the plant genome. J. Schell, Science, 237:1176-83 (1987). Also suitable for use is a plant artificial chromosome, as described in, e.g., U.S. Pat. No. 6,900,012.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol., 1991: 173(1): 86-93; Alpuche-Aranda et al., PNAS, 1992; 89(21): 10079-83), a nirB promoter (Harborne et al. (1992) Mol. Micro. 6:2805-2813), and the like (see, e.g., Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spv promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mol. Microbiol. 22:367-378); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SPI6 promoter (see, e.g., Melton et al. (1984) Nucl. Acids Res. 12:7035-7056); and the like. Suitable strong promoters for use in prokaryotes such as Escherichia coli include, but are not limited to Trc, Tac, T5, T7, and P_{Lambda}. Non-limiting examples of operators for use in bacterial host cells include a lactose promoter operator (LacI repressor protein changes conformation when contacted with lactose, thereby preventing the LacI repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, for example, deBoer et al. (1983) Proc. Natl. Acad. Sci. U.S.A. 80:21-25.)

Non-limiting examples of suitable constitutive promoters for use in prokaryotic host cells include a sigma70 promoter (for example, a consensus sigma70 promoter). Non-limiting examples of suitable inducible promoters for use in bacterial host cells include the pL of bacteriophage λ; Plac; Ptrp; Ptac (Ptrp-lac hybrid promoter); an isopropyl-beta-D44 thiogalactopyranoside (IPTG)-inducible promoter, for example, a lacZ promoter; a tetracycline inducible promoter; an arabinose inducible promoter, for example, PBAD (see, for example, Guzman et al. (1995) J. Bacteriol. 177:4121-4130); a xylose-inducible promoter, for example, Pxyl (see, for example, Kim et al. (1996) Gene 181:71-76); a GAL1 promoter; a tryptophan promoter; a lac promoter; an alcohol-inducible promoter, for example, a methanol-inducible promoter, an ethanol-inducible promoter; a raffinose-inducible promoter; a heat-inducible promoter, for example, heat inducible lambda PL promoter; a promoter controlled by a heat-sensitive repressor (for example, CI857-repressed lambda-based expression vectors; see, for example, Hoffmann et al. (1999) FEMS Microbiol Lett. 177(2):327-34); and the like.

### Expression Vectors

Suitable expression vectors include any of a variety of expression vectors available in the art; and variant and derivatives of such vectors. Those of ordinary skill in the art are familiar with selecting appropriate expression vectors for a given application. Numerous suitable expression vectors are known to those of skill in the art, and many are commercially available. Suitable expression vectors for use in constructing the subject host cells include, but are not limited to, baculovirus vectors, bacteriophage vectors, plasmids, phagemids, cosmids, fosmids, bacterial artificial chromosomes, viral vectors (for example, viral vectors based on vaccinia virus, poliovirus, adenovirus, adeno-associated virus, SV40, herpes simplex virus, and the like), P1-based artificial chromosomes, yeast plasmids, yeast artificial chromosomes, and other vectors. A typical expression vector contains an origin of replication that ensures propagation of the vector, a nucleic acid sequence that encodes a desired enzyme, and one or more regulatory elements that control the synthesis of the desired enzyme.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, an expression vector can be constructed to yield a desired level of copy numbers of the vector. In some embodiments, an expression vector provides for at least 10, between 10 to 20, between 20-50, between 50 and 100, or more than 100 copies of the expression vector in the host cell. Low copy number plasmids generally provide fewer than about 20 plasmid copies per cell; medium copy number plasmids generally provide from about 20 plasmid copies per cell to about 50 plasmid copies per cell, or from about 20 plasmid copies per cell to about 80 plasmid copies per cell; and high copy number plasmids generally provide from about 80 plasmid copies per cell to about 200 plasmid copies per cell, or more than 200 plasmid copies per cell.

Suitable low-copy (centromeric) expression vectors for yeast include, but are not limited to, pRS415 and pRS416 (Sikorski & Hieter (1989) Genetics 122:19-27). In some embodiments, the enzyme-encoding sequences are present on one or more medium copy number plasmids. Medium copy number plasmids generally provide from about 20 plasmid copies per cell to about 50 plasmid copies per cell, or from about 20 plasmid copies per cell to about 80 plasmid copies per cell. Medium copy number plasmids for use in yeast include, e.g., Yep24. In some embodiments, the enzyme-encoding sequences are present on one or more high copy number plasmids. High copy number plasmids generally provide from about 30 plasmid copies per cell to about 200 plasmid copies per cell, or more. Suitable high-copy 2 micron expression vectors in yeast include, but are not limited to, pRS420 series vectors, e.g., pRS425 and pRS426 (Christianson et al. (1992) Gene 110:119-122).

Exemplary low copy expression vectors for use in prokaryotes such as Escherichia coli include, but are not limited to, pACYC184, pBeloBac11, pBR332, pBAD33, pBBRIMCS and its derivatives, pSC101, SuperCos (cosmid), and pWE15 (cosmid). Suitable medium copy expression vectors for use in prokaryotes such as Escherichia coli include, but are not limited to pTrc99A, pBAD24, and vectors containing a ColE1 origin of replication and its derivatives. Suitable high copy number expression vectors for use in prokaryotes such as Escherichia coli include, but are not limited to, pUC, pBluescript, pGEM, and pTZ vectors.

The level of translation of a nucleotide sequence in a genetically modified host cell can be altered in a number of ways, including, but not limited to, increasing the stability of the mRNA, modifying the sequence of the ribosome binding site, modifying the distance or sequence between the ribosome binding site and the start codon of the enzyme coding sequence, modifying the entire intercistronic region located “upstream of” or adjacent to the 5′ side of the start codon of the enzyme coding region, stabilizing the 3′-end of the mRNA transcript using hairpins and specialized sequences, modifying the codon usage of enzyme, altering expression of rare codon tRNAs used in the biosynthesis of the enzyme, and/or increasing the stability of the enzyme, as, for example, via mutation of its coding sequence. Determination of preferred codons and rare codon tRNAs can be based on a survey of genes derived from the host cell.

The expression vector can also contain one or more selectable marker genes that, upon expression, confer one or more phenotypic traits useful for selecting or otherwise identifying host cells that carry the expression vector. Non-limiting examples of suitable selectable markers for prokaryotic cells include resistance to an antibiotic such as tetracycline, ampicillin, chloramphenicol, carbenicillin, or kanamycin.

In some embodiments, instead of antibiotic resistance as a selectable marker for the expression vector, a subject method will employ host cells that do not require the use of an antibiotic resistance conferring selectable marker to ensure plasmid (expression vector) maintenance. In these embodiments, the expression vector contains a plasmid maintenance system such as the 60-kb IncP (RK2) plasmid, optionally together with the RK2 plasmid replication and/or segregation system, to effect plasmid retention in the absence of antibiotic selection (see, for example, Sia et al. (1995) J. Bacteriol. 177:2789-97; Pansegrau et al. (1994) J. Mol. Biol. 239:623-63). A suitable plasmid maintenance system for this purpose is encoded by the parDE operon of RK2, which codes for a stable toxin and an unstable antitoxin. The antitoxin can inhibit the lethal action of the toxin by direct protein-protein interaction. Cells that lose the expression vector that harbors the parDE operon are quickly deprived of the unstable antitoxin, resulting in the stable toxin then causing cell death. The RK2 plasmid replication system is encoded by the trfA gene, which codes for a DNA replication protein. The RK2 plasmid segregation system is encoded by the parCBA operon, which codes for proteins that function to resolve plasmid multimers that may arise from DNA replication.

To generate a genetically modified host cell, one or more heterologous nucleic acids is introduced stably or transiently into a parent host cell, using established techniques, including, but not limited to, electroporation, calcium phosphate precipitation, DEAE-dextran mediated transfection, liposome-mediated transfection, and the like. For stable transformation, a nucleic acid will generally further include a selectable marker, e.g., any of several well-known selectable markers such as neomycin resistance, ampicillin resistance, tetracycline resistance, chloramphenicol resistance, kanamycin resistance, and the like. Stable transformation can also be effected (e.g., selected for) using a nutritional marker gene that confers prototrophy for an essential amino acid such as URA3, HIS3, LEU2, MET2, LYS2 and the like.

### Codon Usage

In some embodiments, a nucleotide sequence used to generate a subject genetically modified host cell for use in a subject method is modified such that the nucleotide sequence reflects the codon preference for the particular host cell. For example, the nucleotide sequence will in some embodiments be modified for yeast codon preference. See, e.g., Bennetzen and Hall (1982) J. Biol. Chem. 257(6): 3026-3031. As another example, in some embodiments, the nucleotide sequence will be modified for E. coli codon preference. See, e.g., Gouy and Gautier (1982) Nucleic Acids Res. 10(22):7055-7074; Eyre-Walker (1996) Mol. Biol. Evol. 13(6):864-872. See also Nakamura et al. (2000) Nucleic Acids Res. 28(1):292.

### Host Cells

The present invention provides genetically modified host cells, e.g., host cells that have been genetically modified with a subject nucleic acid or a subject recombinant vector. In many embodiments, a subject genetically modified host cell is an in vitro host cell. In other embodiments, a subject genetically modified host cell is an in vivo host cell. In other embodiments, a subject genetically modified host cell is part of a multicellular organism.

Host cells are in many embodiments unicellular organisms, or are grown in in vitro culture as single cells. In some embodiments, the host cell is a eukaryotic cell. Suitable eukaryotic host cells include, but are not limited to, yeast cells, insect cells, plant cells, fungal cells, and algal cells. Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Chlamydomonas reinhardtii, and the like. In some embodiments, the host cell is a eukaryotic cell other than a plant cell.

In other embodiments, the host cell is a plant cell. Plant cells include cells of monocotyledons (“monocots”) and dicotyledons (“dicots”).

In other embodiments, the host cell is a prokaryotic cell. Suitable prokaryotic cells include, but are not limited to, any of a variety of laboratory strains of Escherichia coli, Lactobacillus sp., Salmonella sp., Shigella sp., and the like. See, e.g., Carrier et al. (1992) J. Immunol. 148:1176-1181; U.S. Pat. No. 6,447,784; and Sizemore et al. (1995) Science 270:299-302. Examples of Salmonella strains which can be employed in the present invention include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter sphaeroides, Rhodobacter capsulatus, Rhodospirillum rubrum, Rhodococcus sp., and the like. In some embodiments, the host cell is Escherichia coli.

In some embodiments, a subject genetically modified host cell is a plant cell. A subject genetically modified plant cell is useful for producing a selected isoprenoid compound in in vitro plant cell culture. Guidance with respect to plant tissue culture may be found in, for example: Plant Cell and Tissue Culture, 1994, Vasil and Thorpe Eds., Kluwer Academic Publishers; and in: Plant Cell Culture Protocols (Methods in Molecular Biology 111), 1999, Hall Eds, Humana Press.

### Compositions Comprising a Subject Genetically Modified Host Cell

The present invention further provides compositions comprising a subject genetically modified host cell. A subject composition comprises a subject genetically modified host cell, and will in some embodiments comprise one or more further components, which components are selected based in part on the intended use of the genetically modified host cell. Suitable components include, but are not limited to, salts; buffers; stabilizers; protease-inhibiting agents; nuclease-inhibiting agents; cell membrane- and/or cell wall-preserving compounds, e.g., glycerol, dimethylsulfoxide, etc.; nutritional media appropriate to the cell; and the like. In some embodiments, the cells are lyophilized.

### Methods of Producing a P450 Modification Product

The present invention provides methods of producing a P450 modification product, generally involving culturing a subject genetically modified host cell in a suitable medium and under suitable conditions to provide for production of a P450 and production of a P450 modification product. In some embodiments, the method is carried out in vitro (e.g., in a living cell cultured in vitro). In some of these embodiments, the host cell is a eukaryotic cell, e.g., a yeast cell. In other embodiments, the host cell is a prokaryotic cell.

A subject genetically modified host cell provides for enhanced production of a P450 modification product, compared to a control, parent host cell. Thus, e.g., production of a P450 modification product is at least about 10%, at least about 20%, at least about 25%, at least about 30%, at least about 40%, at least about 50%, at least about 60%, at least about 70%, at least about 80%, at least about 90%, at least about 100% (or two-fold), at least about 2.5-fold, at least about 3-fold, at least about 5-fold, at least about 7-fold, at least about 10-fold, at least about 15-fold, at least about 20-fold, at least about 50-fold, at least about 10²-fold, at least about 500-fold, at least about 10³-fold, at least about 5×10³-fold, or at least about 10⁴-fold, or more, higher in the genetically modified host cell, compared to the level of the product produced in a control parent host cell. In some embodiments, a control parent host cell is one that does not comprise the genetic modification(s) that provide for modified levels of one or more P450 activity enhancing gene products.

In some embodiments, a subject method provides for production of a P450-catalyzed modification product in an amount of from about 10 mg/L to about 50 g/L, e.g., from about 10 mg/L to about 25 mg/L, from about 25 mg/L to about 50 mg/L, from about 50 mg/L to about 75 mg/L, from about 75 mg/L to about 100 mg/L, from about 100 mg/L to about 250 mg/L, from about 250 mg/L to about 500 mg/L, from about 500 mg/L to about 750 mg/L, from about 750 mg/L to about 1000 mg/L, from about 1 g/L to about 1.2 g/L, from about 1.2 g/L to about 1.5 g/L, from about 1.5 g/L to about 1.7 g/L, from about 1.7 g/L to about 2 g/L, from about 2 g/L to about 2.5 g/L, from about 2.5 g/L to about 5 g/L, from about 5 g/L to about 10 g/L, from about 10 g/L to about 20 g/L, from about 20 g/L to about 30 g/L, from about 30 g/L to about 40 g/L, or from about 40 g/L to about 50 g/L, or more.

A subject genetically modified host cell can be cultured in vitro in a suitable medium and at a suitable temperature. The temperature at which the cells are cultured is generally from about 18° C. to about 40° C., e.g., from about 18° C. to about 20° C., from about 20° C. to about 25° C., from about 25° C. to about 30° C., from about 30° C. to about 35° C., or from about 35° C. to about 40° C. (e.g., at about 37° C.).

In some embodiments, a subject genetically modified host cell is cultured in a suitable medium (e.g., Luria-Bertoni broth, optionally supplemented with one or more additional agents, such as an inducer (e.g., where a nucleotide sequence encoding a gene product is under the control of an inducible promoter)); and the P450 modification product is isolated from the cell culture medium and/or from cell lysates. In some embodiments, where one or more nucleotide sequences are operably linked to an inducible promoter, an inducer is added to the culture medium; and, after a suitable time, the P450 modification product is isolated from the organic layer overlaid on the culture medium.

In some embodiments, a subject genetically modified host cell is cultured in a suitable medium (e.g., Luria-Bertoni broth), supplemented with 6-amino levulinic acid (ALA). When ALA is present in the culture medium, it can be present at a concentration of from about 25 mg/L to about 200 mg/L, from about 25 mg/L to about 50 mg/L, from about 50 mg/L to about 60 mg/L, from about 60 mg/L to about 70 mg/L, from about 70 mg/L to about 100 mg/L, from about 100 mg/L to about 125 mg/L, from about 125 mg/L to about 150 mg/L, from about 150 mg/L to about 175 mg/L, or from about 175 mg/L to about 200 mg/L.

In some embodiments, a subject genetically modified host cell is cultured in a suitable medium and the culture medium is overlaid with an organic solvent, e.g. dodecane, forming an organic layer. The P450 modification product produced by the genetically modified host cell partitions into the organic layer, from which it can be purified.

In some embodiments, the P450 modification product will be separated from other products, macromolecules, etc., which may be present in the cell culture medium, the cell lysate, or the organic layer. Separation of the P450 modification product from other products that may be present in the cell culture medium, cell lysate, or organic layer is readily achieved using, e.g., standard chromatographic techniques. Separation of the P450 modification product from other products that may be present in the cell culture medium, cell lysate, or organic layer is readily achieved using, e.g., standard isolation techniques for small molecule products. For example, a method can involve pH adjustment and crystallization in organic solvent. Methods of isolating and purifying artemisinin, e.g., are known in the art; see, e.g., U.S. Pat. No. 6,685,972.

In some embodiments, a P450 modification product synthesized by a subject method is further chemically modified in one or more cell-free reactions.

In some embodiments, the P450 modification product is pure, e.g., at least about 40% pure, at least about 50% pure, at least about 60% pure, at least about 70% pure, at least about 80% pure, at least about 90% pure, at least about 95% pure, at least about 98%, or more than 98% pure, where “pure” in the context of a P450 modification product refers to a P450 modification product that is free from other P450 modification products, macromolecules, contaminants, etc.

In some embodiments, the P450 modification product is an artemisinin precursor (e.g., artemisinic alcohol, artemisinic aldehyde, artemisinic acid, etc.). In some of these embodiments, the artemisinin precursor product is pure, e.g., at least about 40% pure, at least about 50% pure, at least about 60% pure, at least about 70% pure, at least about 80% pure, at least about 90% pure, at least about 95% pure, at least about 98%, or more than 98% pure, where “pure” in the context of an artemisinin precursor refers to an artemisinin precursor that is free from side products, macromolecules, contaminants, etc.

### Substrates of a Cytochrome P450 Enzyme

As noted above, a substrate of a cytochrome P450 enzyme is an intermediate in a biosynthetic pathway. Exemplary intermediates include, but are not limited to, isoprenoid precursors; alkaloid precursors; phenylpropanoid precursors; flavonoid precursors; steroid precursors; polyketide precursors; macrolide precursors; sugar alchohol precursors; phenolic compound precursors; and the like. See, e.g., Hwang et al. ((2003) Appl. Environ. Microbiol. 69:2699-2706; Facchini et al. ((2004) TRENDS Plant Sci. 9:116.

Biosynthetic pathway products of interest include, but are not limited to, isoprenoid compounds, alkaloid compounds, phenylpropanoid compounds, flavonoid compounds, steroid compounds, polyketide compounds, macrolide compounds, sugar alcohols, phenolic compounds, and the like.

Alkaloid compounds are a large, diverse group of natural products found in about 20% of plant species. They are generally defined by the occurrence of a nitrogen atom in an oxidative state within a heterocyclic ring. Alkaloid compounds include benzylisoquinoline alkaloid compounds, indole alkaloid compounds, isoquinoline alkaloid compounds, and the like. Alkaloid compounds include monocyclic alkaloid compounds, dicyclic alkaloid compounds, tricyclic alkaloid compounds, tetracyclic alkaloid compounds, as well as alkaloid compounds with cage structures. Alkaloid compounds include: 1) Pyridine group: piperine, coniine, trigonelline, arecaidine, guvacine, pilocarpine, cytisine, sparteine, pelletierine; 2) Pyrrolidine group: hygrine, nicotine, cuscohygrine; 3) Tropine group: atropine, cocaine, ecgonine, pelletierine, scopolamine; 4) Quinoline group: quinine, dihydroquinine, quinidine, dihydroquinidine, strychnine, brucine, and the veratrum alkaloids (e.g., veratrine, cevadine); 5) Isoquinoline group: morphine, codeine, thebaine, papaverine, narcotine, narceine, hydrastine, and berberine; 6) Phenethylamine group: methamphetamine, mescaline, ephedrine; 7) Indole group: tryptamines (e.g., dimethyltryptamine, psilocybin, serotonin), ergolines (e.g., ergine, ergotamine, lysergic acid, etc.), and beta-carbolines (e.g., harmine, yohimbine, reserpine, emetine); 8) Purine group: xanthines (e.g., caffeine, theobromine, theophylline); 9) Terpenoid group: aconite alkaloids (e.g., aconitine), and steroids (e.g., solanine, samandarin); 10) Betaine group: (quaternary ammonium compounds: e.g., muscarine, choline, neurine); and 11) Pyrazole group: pyrazole, fomepizole. Exemplary alkaloid compounds are morphine, berberine, vinblastine, vincristine, cocaine, scopolamine, caffeine, nicotine, atropine, papaverine, emetine, quinine, reserpine, codeine, serotonin, etc. See, e.g., Facchini et al. ((2004) Trends Plant Science 9:116).

### Substrates of Isoprenoid-Modifying Enzymes

The term “isoprenoid precursor compound” is used interchangeably with “isoprenoid precursor substrate” to refer to a compound that is a product of the reaction of a terpene synthase on a polyprenyl diphosphate. The product of action of a terpene synthase (also referred to as a “terpene cyclase”) reaction is the so-called “terpene skeleton.” In some embodiments, the isoprenoid-modifying enzyme catalyzes the modification of a terpene skeleton, or a downstream product thereof. Thus, in some embodiments, the isoprenoid precursor is a terpene skeleton. Isoprenoid precursor substrates of an isoprenoid precursor-modifying enzyme include monoterpenes, diterpenes, triterpenes, and sesquiterpenes.

Monoterpene substrates of an isoprenoid-modifying enzyme encoded by a subject nucleic acid include, but are not limited to, any monoterpene substrate that yields an oxidation product that is a monoterpene compound or is an intermediate in a biosynthetic pathway that gives rise to a monoterpene compound. Exemplary monoterpene substrates include, but are not limited to, monoterpene substrates that fall into any of the following families: Acyclic monoterpenes, Dimethyloctanes, Menthanes, Irregular Monoterpenoids, Cineols, Camphanes, Isocamphanes, Monocyclic monoterpenes, Pinanes, Fenchanes, Thujanes, Caranes, lonones, Iridanes, and Cannabanoids. Exemplary monoterpene substrates, intermediates, and products include, but are not limited to, limonene, citranellol, geraniol, menthol, perillyl alcohol, linalool, and thujone.

Diterpene substrates of an isoprenoid-modifying enzyme encoded by a subject nucleic acid include, but are not limited to, any diterpene substrate that yields an oxidation product that is a diterpene compound or is an intermediate in a biosynthetic pathway that gives rise to a diterpene compound. Exemplary diterpene substrates include, but are not limited to, diterpene substrates that fall into any of the following families: Acyclic Diterpenoids, Bicyclic Diterpenoids, Monocyclic Diterpenoids, Labdanes, Clerodanes, Taxanes, Tricyclic Diterpenoids, Tetracyclic Diterpenoids, Kaurenes, Beyerenes, Atiserenes, Aphidicolins, Grayanotoxins, Gibberellins, Macrocyclic Diterpenes, and Elizabethatrianes. Exemplary diterpene substrates, intermediates, and products include, but are not limited to, casbene, eleutherobin, paclitaxel, prostratin, and pseudopterosin.

Triterpene substrates of an isoprenoid-modifying enzyme encoded by a subject nucleic acid include, but are not limited to, any triterpene substrate that yields an oxidation product that is a triterpene compound or is an intermediate in a biosynthetic pathway that gives rise to a triterpene compound. Exemplary triterpene substrates, intermediates, and products include, but are not limited to, arbrusideE, bruceantin, testosterone, progesterone, cortisone, and digitoxin.

Sesquiterpene substrates of an isoprenoid-modifying enzyme encoded by a subject nucleic acid include, but are not limited to, any sesquiterpene substrate that yields an oxidation product that is a sesquiterpene compound or is an intermediate in a biosynthetic pathway that gives rise to a sesquiterpene compound. Exemplary sesquiterpene substrates include, but are not limited to, sesquiterpene substrates that fall into any of the following families: Farnesanes, Monocyclofarnesanes, Monocyclic sesquiterpenes, Bicyclic sesquiterpenes, Bicyclofarnesanes, Bisbolanes, Santalanes, Cupranes, Herbertanes, Gymnomitranes, Trichothecanes, Chamigranes, Carotanes, Acoranes, Antisatins, Cadinanes, Oplopananes, Copaanes, Picrotoxanes, Himachalanes, Longipinanes, Longicyclanes, Caryophyllanes, Modhephanes, Siphiperfolanes, Humulanes, Intergrifolianes, Lippifolianes, Protoilludanes, Illudanes, Hirsutanes, Lactaranes, Sterpuranes, Fomannosanes, Marasmanes, Germacranes, Elemanes, Eudesmanes, B akkanes, Chilosyphanes, Guaianes, Pseudoguaianes, Tricyclic sesquiterpenes, Patchoulanes, Trixanes, Aromadendranes, Gorgonanes, Nardosinanes, Brasilanes, Pinguisanes, Sesquipinanes, Sesquicamphanes, Thujopsanes, Bicylcohumulanes, Alliacanes, Sterpuranes, Lactaranes, Africanes, Integrifolianes, Protoilludanes, Aristolanes, and Neolemnanes. Exemplary sesquiterpene substrates include, but are not limited to, amorphadiene, alloisolongifolene, (−)-α-trans-bergamotene, (−)-β-elemene, (+)-germacrene A, germacrene B, (+)-γ-gurjunene, (+)-ledene, neointermedeol, (+)-β-selinene, and (+)-valencene.

A subject method is useful for production of a variety of isoprenoid compounds, including, but not limited to, artemisinic acid (e.g., where the sesquiterpene substrate is amorpha-4,11-diene), alloisolongifolene alcohol (e.g., where the substrate is alloisolongifolene), (E)-trans-bergamota-2,12-dien-14-ol (e.g., where the substrate is (−)-α-trans-bergamotene), (−)-elema-1,3,11(13)-trien-12-ol (e.g., where the substrate is (−)—β-elemene), germacra-1(10),4,11(13)-trien-12-ol (e.g., where the substrate is (+)-germacrene A), germacrene B alcohol (e.g., where the substrate is germacrene B), 5,11(13)-guaiadiene-12-ol (e.g., where the substrate is (+)-γ-gurjunene), ledene alcohol (e.g., where the substrate is (+)-ledene), 4β-H-eudesm-11(13)-ene-4,12-diol (e.g., where the substrate is neointermedeol), (+)-β-costol (e.g., where the substrate is (+)-β-selinene, and the like; and further derivatives of any of the foregoing.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Identification of Candidate Genes for Modulation

Amorphadiene oxidase (AMO) is a P450 isolated from Artemisia annua that can be used for a key transformation in the semisynthesis of artemisinin, an important antimalarial drug. AMO converts amorphadiene into artemisinic acid in three oxidative steps and requires O₂, NADPH, and a P450 reductase (CPR) redox partner. In E. coli, artemisinic acid can be produced at titers of 105±10 mg/L. This example shows identification of genes that affect artemisinic acid production.

Generation of pAM92

Expression plasmid pAM36-MevT66 was generated by inserting the MevT66 operon into the pAM36 vector. The pAM36 vector was generated by inserting an oligonucleotide cassette containing AscI-SfiI-AsiSI-XhoI-PacI-FsIl-PmeI restriction sites into the pACYC 184 vector (GenBank accession number X06403), and by removing the tetracycline resistance conferring gene in pACYCI84. The MevT66 operon encodes the set of MEV pathway enzymes that together transform the ubiquitous precursor acetyl-CoA to (R)-mevalonate, namely acetoacetyl-CoA thiolase, HMG-CoA synthase, and HMG-CoA reductase. The operon was synthetically generated and comprises the atoB gene from Escherichia coli (GenBank accession number NC_{—}000913 REGION: 2324131.2325315), the ERG13 gene from Saccharomyces cerevisiae (GenBank accession number X96617, REGION: 220.1695), and a truncated version of the HMG1 gene from Saccharomyces cerevisiae (GenBank accession number M22002, REGION: 1777.3285), all three sequences being codon-optimized for expression in Escherichia coli. The synthetically generated MevT66 operon was flanked by a 5′ EcoRI restriction site and a 3′ Hind III restriction site, and could thus be cloned into compatible restriction sites of a cloning vector such as a standard pUC or pACYC origin vector. From this construct, the MevT66 operon was PCR amplified with flanking SfiI and AsiSI restriction sites, the amplified DNA fragment was digested to completion using SfiI and AsiSI restriction enzymes, the reaction mixture was resolved by gel electrophoresis, the approximately 4.2 kb DNA fragment was gel extracted using a gel purification kit (Qiagen, Valencia, Calif.), and the isolated DNA fragment was ligated into the SfiI AsiSI restriction site of the pAM36 vector, yielding expression plasmid pAM36-MevT66.

Expression plasmid pMBI was generated by inserting the MBI operon into the pBBR1MCS-3 vector. In addition to the enzymes of the MevB operon, the MBI operon also encodes an isopentenyl pyrophosphate isomerase, which catalyzes the conversion of IPP to DMAPP. The MBI operon was generated by PCR amplifying from Escherichia coli genomic DNA the coding sequence of the idi gene (GenBank accession number AF119715) using primers that contained an XmaI restriction site at their 5′ ends, digesting the amplified DNA fragment to completion using XmaI restriction enzyme, resolving the reaction mixture by gel electrophoresis, gel extracting the approximately 0.5 kb fragment, and ligating the isolated DNA fragment into the XmaI restriction site of expression plasmid pMevB-Cm, thereby placing idi at the 3′ end of the MevB operon. The MBI operon was subcloned into the SalI SacI restriction site of vector pBBRIMCS-3 (Kovach et al., Gene 166(1): 175-176 (1995)), yielding expression plasmid pMBI (see U.S. Pat. No. 7,192,751). Expression plasmid pMBIS was generated by inserting the ispA gene into pMBI. The ispA gene encodes a farnesyl pyrophosphate synthase, which catalyzes the condensation of two molecules of IPP with one molecule of DMAPP to make farnesyl pyrophosphate (FPP). The coding sequence of the ispA gene (GenBank accession number D00694, REGION: 484.1383) was PCR amplified from Escherichia coli genomic DNA using a forward primer with a SacII restriction site and a reverse primer with a SacI restriction site. The amplified PCR product was digested to completion using SacII and SacI restriction enzymes, the reaction mixture was resolved by gel electrophoresis, and the approximately 0.9 kb DNA fragment was gel extracted, and the isolated DNA fragment was ligated into the SacII SacI restriction site of pMBI, thereby placing the ispA gene 3′ of idi and the MevB operon, and yielding expression plasmid pMBIS (see U.S. Pat. No. 7,192,751; and SEQ ID NO:4 of U.S. Pat. No. 7,183,089). Expression plasmid pAM45 was generated by inserting the MBIS operon into pAM36-MevT66 and adding lacUV5 promoters in front of the MBIS and MevT66 operons. The MBIS operon was PCR amplified from pMBIS using primers comprising a 5′ XhoI restriction site and a 3′ PacI restriction site, the amplified PCR product was digested to completion using XhoI and PacI restriction enzymes, the reaction mixture was resolved by gel electrophoresis, the approximately 5.4 kb DNA fragment was gel extracted, and the isolated DNA fragment was ligated into the XhoI PacI restriction site of pAM36-MevT66, yielding expression plasmid pAM43. A DNA fragment comprising a nucleotide sequence encoding the lacUV5 promoter was synthesized from oligonucleotides, and sub-cloned into the AscI SfiI and AsiSI XhoI restriction sites of pAM43, yielding expression plasmid pAM45.

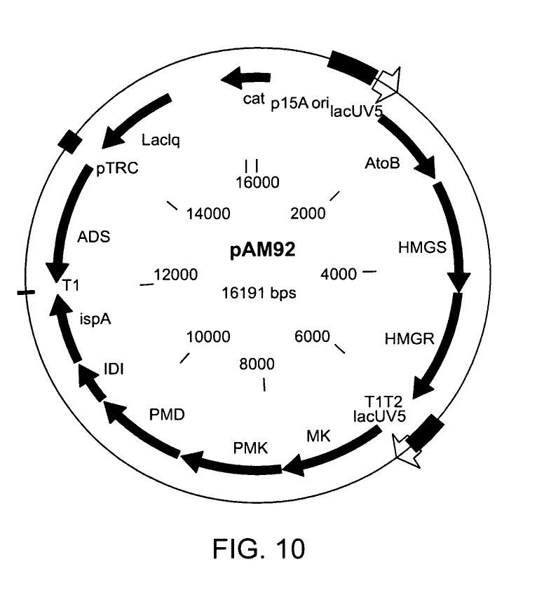

Expression plasmid pAM92 was generated by inserting a nucleotide sequence encoding an amorpha-4,11-diene synthase (“ADS”) into pAM45. The nucleotide sequence encoding ADS was designed such that upon translation the amino acid sequence of the enzyme would be identical to that described by Merke et al. (2000) Ach. Biochem. Biophys. 381:173-180. The nucleotide sequence encoding ADS was codon-optimized for expression in Escherichia coli (see U.S. Pat. No. 7,192,751). The nucleotide sequence of pAM92 is given as SEQ ID NO:70. A plasmid map of pAM92 is shown in FIG. 10.

### Results

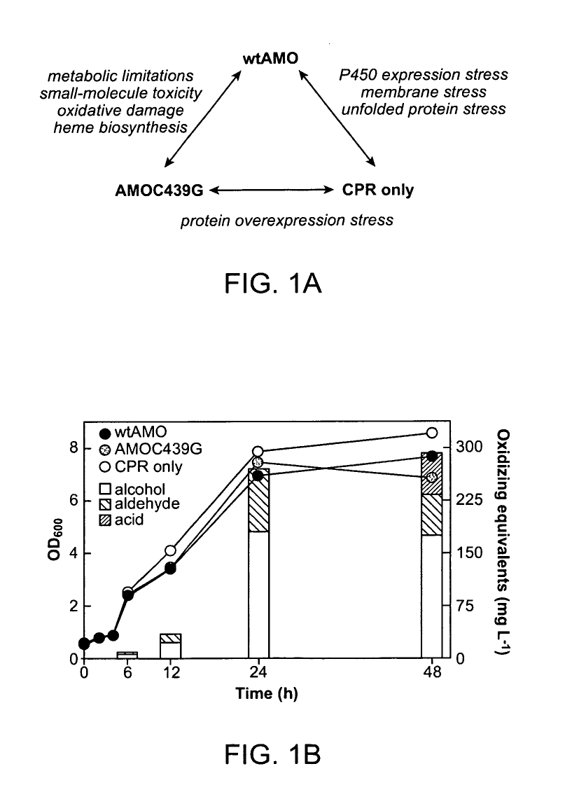

To build an improved host for in vivo production of small molecules involving P450s, DNA microarray studies were used to pinpoint cellular responses and limitations resulting from P450 expression and/or in vivo P450 oxidation chemistry. A three-way comparison was carried out in order to isolate the effects of both P450 expression as well as P450 turnover (FIG. 1A). E. coli DH1 was co-transformed with pAM92, a plasmid which provides the amorphadiene substrate, as well as a second plasmid containing amorphadiene oxidase (A13sAMO) and its CPR partner (ctAACPR). Three different versions of the AMO plasmid were used—pBAD24-A13sAMO-ctAACPR (wtAMO), pBAD24-A13sAMOC439G (AMOC439G, wt numbering), and pBAD24-ctAACPR(CPR only) (FIG. 1A). The C439G mutation eliminates the heme ligand of AMO, thereby retaining AMO expression but knocking out activity with a single point mutation. The CPR only construct eliminates both AMO expression and activity. The three strains were inoculated into TB containing chloramphenicol (50 mg/L) and carbenicillin (50 mg/L) and grown in parallel at 30° C. in 2 L shake flasks at 150 rpm. At a cell density of OD_{600 nm}=0.5, the cultures were induced with 0.5 mM IPTG and 0.2% arabinose and the heme supplement δ-aminolevulinic acid was added to 65 mg/L. The growth temperature was also dropped to 20° C. at this time. Cells were collected before induction (T₀) as well as 6 h (T₁), 12 h (T₂), 24 h (T₃) and 48 h (T₄) post-induction. These samples were characterized for AMO expression by Western blot and the wtAMO sample was analyzed for product formation by GC-MS (FIG. 1B).

FIGS. 1A and 1B. Measuring the transcriptional response of E. coli to P450 expression and turnover. (A) A 3-way comparison between wtAMO, C439 mutant, and CPR only strains allows isolation of different responses related to both turnover as well as protein expression. (B) Growth curves and production titers of different strains.

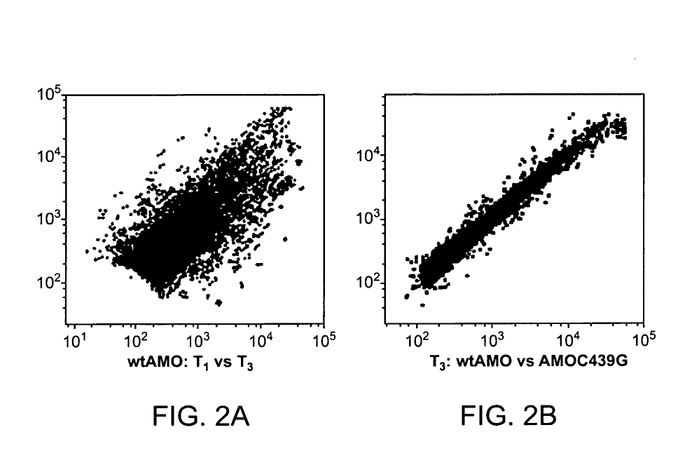

The T₃ sample was selected for initial comparison because product analysis shows that this is the first timepoint in which a significant number of AMO turnovers have taken place. RNA was isolated from wtAMO T₀ and T₃, AMOC439G T₃, and CPR only T₃ samples. Three comparisons of transcripts were carried out in triplicate: (1) wtAMO T₀: wtAMO T₃, (2) wtAMO T₃: AMOC439GT₃, (3) wtAMOT₃: CPR only T₃. This coverage made it possible to address several points in developing a picture of the metabolic state of E. coli when expressing active P450s. Comparison 1 shows the change in transcriptional activity upon induction of the P450 and CPR in the wtAMO strain (FIG. 2A). Clearly, many differential responses were observed but the majority is unrelated to AMO activity and/or expression. A targeted comparison of wtAMO and AMOC439G at T₃ in which only activity is removed shows a much higher correlation in gene expression with a very select set of responses (FIG. 2B). The major responses observed are related to membrane stress (oxidative stress, osmotic stress), oxidative stress (OxyR regulon), protein overexpression stress (heat shock response), as well as some indications of upregulation of heme biosynthesis, iron and sulfur assimilation, and the pentose phosphate pathway for NADPH production.

FIGS. 2A and 2B. Comparison of transcripts in AMO strains. (A) Pre- and post-induction of wtAMO, and (B) Comparison of wtAMO and AMOC439A at T₃.

### Example 2

### Modulating Expression of Candidate Genes and the Effect on E. Coli Physiology and/or Titers of Small Molecule Products

The effect of overexpression of the groES/groEL chaperone proteins on in vivo activity of P450s was examined. Co-expression of groES/groEL with AMO led to overall lower protein expression as visualized by Western blots (FIG. 3A), however turnover numbers of AMO were maintained with lower protein (FIG. 3B). These results indicate that the specific activity of AMO has been improved in vivo with co-expression of protein chaperones.

FIGS. 3A and 3B. Effect of chaperone co-expression on AMO in vivo productivity. (A) Western blot showing AMO expression without (A13-AMO) and with (GroEL/ES) chaperone co-expression using the pCWOri expression vector. (B) Production of the alcohol and aldehyde products of AMO in various vector systems (pBAD24, pCWOri, pTrc99a) without (−) and with (+) chaperone co-expression.

### Example 3

### Effect of Co-Expression of Various Genes on AMO Turnover

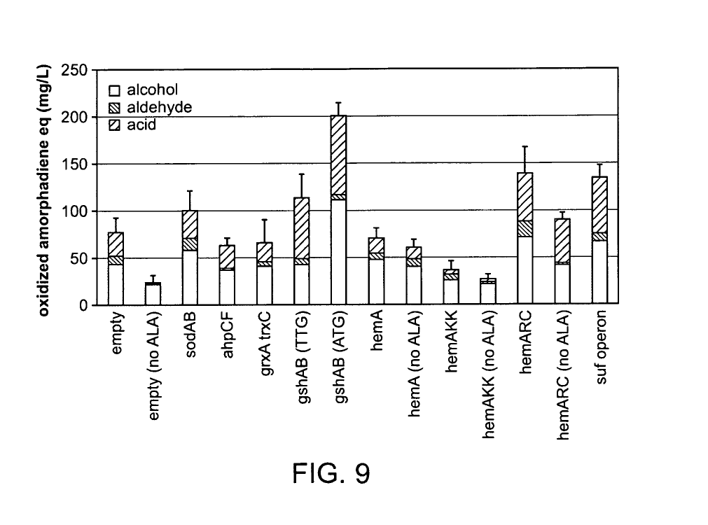

The effect of gene co-expression on AMO turnover, as measured by oxidized amorphadiene equivalents, was examined. FIG. 9 depicts the effect of oxidative stress-related genes on AMO turnover. E. coli were transformed with pAM92 and pBAD24-A13sAMO-ctAACPR, as described above, and further genetically modified with a plasmid comprising a nucleotide sequence encoding an oxidative stress-related gene product. Cells were cultured in the presence or absence of 65 mg/L 6-amino levulinic acid (ALA), as described above.

Oxidative stress-related genes include those involved in management of cellular redox state (sodAB, grxA, trxC, gshAB); iron-sulfur cluster repair (suf operon: sufACBDS); repair of lipid peroxides (ahpCF); and metabolic limitations related to heme biosynthesis (e.g., hemA from E. coli; hemARC, from R. capsulatus), as shown in FIG. 9. In FIG. 9, “Empty” indicates negative control of the empty co-expression plasmid with no additional gene expressed; “gshAB (TTG)” indicates that the “TTG” start codon present in native E. coli gshA was used in the construct; “gshAB (ATG)” indicates that the “TTG” start codon present in native E. coli gshA was changed to an “ATG” codon; and “hemARC” indicates that the hemA sequence of Rhodobacter capsulatus was used.

The data presented in FIG. 9 show that, when co-expressed with pAM92, the following oxidative stress-related gene products provided for an increased production level of oxidized amorphadiene: 1) gshAB (when the native TTG start codon was changed to an ATG start codon); 2) hemA (when the R. capsulatus sequence was used); and 3) suf operon-encoded polypeptides.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified host cell, wherein said genetically modified host cell comprises a nucleic acid comprising a nucleotide sequence encoding an oxidative stress-related gene product, wherein production of the oxidative stress-related gene product provides for increased production of an isoprenoid or isoprenoid precursor by the genetically modified host cell, compared to a control host cell not genetically modified with the nucleic acid.

2. The genetically modified host cell of claim 1, wherein the genetically modified host cell is a prokaryotic cell.

3. The genetically modified host cell of claim 1, wherein the genetically modified host cell is a eukaryotic cell.

4. The genetically modified host cell of claim 1, wherein the isoprenoid or isoprenoid precursor is produced by the cell in a recoverable amount of at least about 100 mg/L on a cell culture basis.

5. The genetically modified host cell of claim 1, wherein said nucleotide sequence encoding said oxidative stress-related gene product encodes a glutamate-cysteine ligase and glutathione synthetase, a δ-aminolevulinic acid synthase, or polypeptides encoded by a suf operon.

6. The genetically modified host cell of claim 5, wherein said oxidative stress-related gene product is a glutamate-cysteine ligase and glutathione synthetase, and where said nucleotide sequence encoding said a glutamate-cysteine ligase and glutathione synthetase comprises a nucleotide sequence having at least about 75% identity to the nucleotide sequence set forth in SEQ ID NO:71.

7. The genetically modified host cell of claim 5, wherein said oxidative stress-related gene product is a 5-aminolevulinic acid synthase, and where said nucleotide sequence encoding said 5-aminolevulinic acid synthase comprises a nucleotide sequence having at least about 75% identity to the nucleotide sequence set forth in SEQ ID NO:20.

8. The genetically modified host cell of claim 1, wherein said oxidative stress-related gene product is encoded by a suf operon, and where said nucleotide sequence comprises a nucleotide sequence having at least about 75% identity to the nucleotide sequence set forth in SEQ ID NO:73.

9. The genetically modified host cell of claim 1, wherein the cytochrome P450 enzyme produced by the cell is a heterologous cytochrome P450 enzyme, and wherein the host cell is further genetically modified with a nucleic acid comprising a nucleotide sequence encoding the heterologous cytochrome P450 enzyme.

10. The genetically modified host cell of claim 1, wherein the host cell is further genetically modified with a nucleic acid comprising a nucleotide sequence encoding a cytochrome P450 reductase.

11. The genetically modified host cell of claim 9, wherein the heterologous cytochrome P450 enzyme is an isoprenoid pathway intermediate-modifying cytochrome P450 enzyme, and wherein the host cell is further genetically modified with one or more nucleic acids comprising nucleotide sequences encoding one or more mevalonate pathway enzymes.

12. The genetically modified host cell of claim 11, wherein the host cell is a prokaryotic host cell that does not normally synthesize isopentenyl pyrophosphate via a mevalonate pathway.

13. A method of producing an isoprenoid or an isoprenoid precursor, the method comprising:
a) culturing the genetically modified host cell of claim 1 in a suitable medium; and
b) recovering the isoprenoid or an isoprenoid precursor.

14. The method of claim 13, further comprising purifying the isoprenoid or an isoprenoid precursor.

15. The method of claim 13, further comprising modifying the isoprenoid or an isoprenoid precursor in a cell-free reaction in vitro.

16. The method of claim 15, wherein the isoprenoid or an isoprenoid precursor is produced by the cell in a recoverable amount of at least about 100 mg/L on a cell culture basis.
